(19) United States
(12) Patent Application Publication
(10) Pub. No.: US 2026/0345646 A1
(43) Pub. Date: Nov. 5, 2026

(54) PHARMACEUTICAL COMPOSITIONS AND METHODS OF USING FOR TREATING NON-ALCOHOLIC FATTY LIVER DISEASE, NASH/MASH, AND LIVER FIBROSIS

(71) Applicant: Viking Therapeutics, Inc., San Diego, CA (US)

(72) Inventors: Brian Lian, San Diego, CA (US); Marianne Mancini, San Diego, CA (US); Becky Steele, San Diego, CA (US); Geoff Barker, San Diego, CA (US); William Hoye, San Diego, CA (US); Maureen Barnes, San Diego, CA (US)

(21) Appl. No.: 19/356,136

(22) Filed: Oct. 12, 2025

Related U.S. Application Data
(63) Continuation of application No. 19/193,412, filed on Apr. 29, 2025.

(51) Int. Cl. A61K 31/665; A61K 47/10; A61P 1/16

(52) U.S. Cl. CPC A61K 31/665; A61K 47/10; A61P 1/16

(57) ABSTRACT

Administering a pharmaceutical composition to a human. In some embodiments, the method statistically significantly reduces and/or maintains liver fat for longer than a twelve-week time period. In some embodiments, the method statistically significantly treats and/or improves fibrosis, non-alcoholic fatty liver disease, and/or NASH/MASH. The pharmaceutical composition may include form(s) of VK2809 and a pharmaceutically acceptable carrier.

Provided herein are novel pharmaceutical compositions that contain at least one form of VK2809, that significantly reduce and/or maintain, over a time period, such as a twelve and/or fifty-two week time period, liver fat and significantly treat and/or improve fibrosis, non-alcoholic fatty liver disease, and steatohepatitis. Also provided herein are methods, and uses of the novel pharmaceutical compositions.

### BACKGROUND

Fatty liver disease is the most common liver disease in the world. Non-alcoholic fatty liver disease (NAFLD) is a spectrum of disease characterized by hepatic steatosis in the absence of excessive alcohol consumption (Di Diehl A M, day C. Cause, pathogenesis and treatment of nonalcoholic steatohepatitis. New Eng J Med. 2017; 377(21): 2063-2072. doi: 10.1056/NEJMra1503519; Hardy T, Oakley F, Anstee Q M, day C P. Nonalcoholic fatty liver disease: pathogenesis and disease spectrum. Annu Rev Pathol. 2016; 11:451-496. doi: 10.1146/annurev-pathol-012615-044224). About 25% of adults in the United States have NAFLD while more than a quarter of adults with NAFLD are estimated to have non-alcoholic steatohepatitis (NASH, also called MASH, metabolic dysfunction associated steatohepatits) (Younossi Z M, Blissett D, Blissett R, et al. The economic and clinical burden of non-alcoholic fatty liver disease in the United States and Europe. Hepatology. 2016; 64(5): 1577-1586. doi: 10.1002/hep.28785). NASH/MASH is an advanced form of fatty liver disease when the fat in the liver becomes toxic, damaging liver cells and making the liver inflamed. As the liver reacts to these changes, it can get scarred leading to fibrosis.

VK2809, a prodrug of VK2809A, is a potent tissue and receptor-subtype selective agonist of the thyroid hormone β receptor (TRB) that is readily converted in the liver to the active metabolite, VK2809A. VK2809 is being developed as a treatment for non-alcoholic steatohepatitis (NASH)/metabolic dysfunction associated steatohepatitis (MASH). NASH and MASH may be used interchangeably herein and are sometimes referred to as NASH/MASH.

During this study, the name for non-alcoholic fatty liver disease (NAFLD) changed to metabolic dysfunction-associated steatotic liver disease (MASLD) and NASH changed to metabolic dysfunction-associated steatohepatitis (MASH) (Rinella M E, Lazarus J V, Ratziu V, et al. A multisociety Delphi consensus statement on new fatty liver disease nomenclature. Hepatology. 2023; 78(6): 1966-1986. doi: 10.1097/HEP.0000000000000520). These changes will be reflected in future patent applications for VK2809. However, in this patent application we will continue to use the terms NAFLD and NASH, sometimes referring to NAFLD/MASLD and also sometimes to NASH/MASH.

Fatty liver disease is the most common liver disease in the world. NAFLD is a spectrum of disease characterized by hepatic steatosis in the absence of excessive alcohol consumption (Di Diehl A M, day C. Cause, pathogenesis and treatment of non-alcoholic steatohepatitis. New Eng J Med. 2017; 377(21): 2063-2072. doi: 10.1056/NEJMra1503519; Hardy T, Oakley F, Anstee Q M, day C P. Nonalcoholic fatty liver disease: pathogenesis and disease spectrum. Annu Rev Pathol. 2016; 11:451-496. doi: 10.1146/annurev-pathol-012615-044224). About 25% of adults in the United States have NAFLD while more than a quarter of adults with NAFLD are estimated to have NASH (Younossi Z M, Blissett D, Blissett R, et al. The economic and clinical burden of nonalcoholic fatty liver disease in the United States and Europe. Hepatology. 2016; 64(5): 1577-1586. doi: 10.1002/hep.28785). In a large study, the presence of NASH was found to be associated with obesity (80%), hyperlipidemia (72%), and type 2 diabetes mellitus (44%), among other metabolic abnormalities (Williams C D, Stengel J, Asike M I, et al. Prevalence of nonalcoholic fatty liver disease and nonalcoholic steatohepatitis among a largely middle-aged population utilizing ultrasound and liver biopsy: a prospective study. Gastroenterology. 2011; 140(1): 124-131. doi: 10.1053/j.gastro.2010.09.038). NASH is strongly associated with the development of liver fibrosis (Adams L A, Harmsen S, St Sauver J L, et al. Nonalcoholic fatty liver disease increases risk of death among patients with diabetes: a community-based arm study. Am J Gastroenterol. 2010; 105(7): 1567-1573. doi: 10.1038/ajg.2010.18; Younossi Z M, Stepanova M, Rafiq N, et al. Pathologic criteria for nonalcoholic steatohepatitis: interprotocol agreement and ability to predict liver-related mortality. Hepatology. 2011; 53(6): 1874-1882. doi: 10.1002/hep.24268; Ekstedt M, Hagstrom H, Nasr P, et al.

Fibrosis stage is the strongest predictor for disease-specific mortality in NAFLD after up to 33 years of follow-up. Hepatology. 2015; 61(5): 1547-1554. doi: 10.1002/hep.27368; Angulo P, Kleiner D E, Dam-Larsen S, et al. Liver fibrosis, but no other histologic features, is associated with long-term outcomes of patients with nonalcoholic fatty liver disease. Gastroenterology. 2015; 149(2): 389-97.e10. doi: 10.1053/j.gastro.2015.04.043), and the histologic severity of fibrosis independently predicts liver-related illness, liver transplantation, and liver-related death in patients with NAFLD (Ekstedt M, Hagstrom H, Nasr P, et al. Fibrosis stage is the strongest predictor for disease-specific mortality in NAFLD after up to 33 years of follow-up. Hepatology. 2015; 61(5): 1547-1554. doi: 10.1002/hep.27368; Angulo P, Kleiner D E, Dam-Larsen S, et al. Liver fibrosis, but no other histologic features are associated with long-term outcomes of patients with nonalcoholic fatty liver disease. Gastroenterology. 2015; 149(2): 389-97.e10. doi: 10.1053/j.gastro.2015.04.043).

Although the rate of progression may vary significantly between individuals, progressive fibrosis in patients with NASH increases the risk of cirrhosis and primary liver cancer (Angulo P, Machado M V, Diehl A M. Fibrosis in nonalcoholic fatty liver disease: mechanisms and clinical implications. Semin Liver Dis. 2015; 35(2): 132-145. doi: 10.1055/s-0035-1550065). In individuals with NASH and cirrhosis, the incidence of hepatocellular carcinoma is 1% to 2% per year (Wong, 2016; Marengo, 2016). NASH is one of the top reasons for liver transplantation in the United States (Charlton M R, Burns J M, Pedersen R A, Watt K D, Heimbach J K, Dierkhising R A. Frequency and outcomes of liver transplantation for nonalcoholic steatohepatitis in the United States. Gastroenterology. 2011; 141(4): 1249-1253. doi: 10.1053/j.gastro.2011.06.061; Wong R J, Aguilar M, Cheung R, et al. Nonalcoholic steatohepatitis is the second leading etiology of liver disease among adults awaiting liver transplantation in the United States. Gastroenterology. 2015; 148(3): 547-555. doi: 10.1053/j.gastro.2014.11.039).

A significant association exists between the presence of NAFLD (particularly NASH) and cardiovascular disease and mortality from cardiovascular disease (Angulo P. Nonalcoholic fatty liver disease. N Engl J Med. 2002; 346(16): 1221-1231. doi: 10.1056/NEJMra011775; Targher G, day C P, Bonora E. Risk of cardiovascular disease in patients with nonalcoholic fatty liver disease. New Engl J Med. 2010; 363(140): 1341-1350. doi: 10.1056/NEJMra0912063). Despite the association with cardiovascular disease, there is no convincing evidence that lipid-lowering agents, including statins, are beneficial for patients with NASH (Browning J D. Statins and hepatic steatosis: perspectives from the Dallas Heart Study. Hepatology. 2006; 44(2): 466-471. doi: 10.1002/hep.21248).

The economic burden of NAFLD in the United States is large and estimated to cost over $100 billion in direct medical costs annually (Younossi Z M, Koenig A B, Abdelatif D, Fazel Y, Henry L, Wymer M. Global epidemiology of nonalcoholic fatty liver disease-meta-analytic assessment of prevalence, incidence, and outcomes. Hepatology. 2016; 64(1): 73-84. doi: 10.1002/hep.28431). Several agents are in development for the treatment of NASH, including obeticholic acid and the TRB agonist MGL-3196 (Connolly J J, Ooka K, Lim J K. Future pharmacotherapy for non-alcoholic steatohepatitis (NASH): review of phase 2 and 3 trials. J Clin Transl Hepatol. 2018; 6(3): 264-275. doi: 10.14218/JCTH.2017.00056).

Thyroid hormone is a key regulator of lipid metabolism, particularly through its activation of the beta isoform of nuclear triiodothyronine (T3) receptor in the liver. Known effects of thyroid receptor activation on lipid metabolism include increased cholesterol metabolism via expression of the enzyme CYP7A1 and decreased de novo lipogenesis through suppressed expression of hepatic sterol regulatory element binding protein-1. These characteristics suggest potential therapeutic benefits in patients with elevated lipids, including hypercholesterolemia, hypertriglyceridemia, and NASH (Sinha R A, Singh B K, Yen P M. Thyroid hormone regulation of hepatic lipid and carbohydrate metabolism. Trends Endocrinol Metab. 2014; 25(10): 538-545. doi: 10.1016/j.tem.2014.07.001).

VK2809 aims to be β receptor selective while minimizing a receptor effects. Thyroid hormone β receptor effects are directed towards the liver and include regulating lipid metabolism, reducing lipids (e.g., LDL-C, triglycerides, atherogenic proteins), and improving metabolic control. Thyroid hormone α receptor effects are directed toward the heart and skeletal muscle and include proarrhythmic potential, elevating heart rate, and bone and/or cartilage effects.

### SUMMARY

In some embodiments, a method for statistically significantly reducing and/or maintaining liver fat for longer than a twelve-week time period, includes administering an amount effective for said statistically reducing and/or maintaining of a pharmaceutical composition to a human during a time the human is in need of such significant reduction and/or maintenance in liver fat, the composition includes: an amorphous form of VK2809; and a pharmaceutically acceptable carrier.

In some embodiments, the amount effective ranges from 1 mg up to and including 2.5 mg of the pharmaceutical composition and said administering occurs once every day. In some embodiments, the amount effective ranges from greater than 2.5 mg up to and including 4 mg of the pharmaceutical composition and said administering occurs once every day. In some embodiments, the amount effective ranges from 3 mg up to and including 3.5 mg of the pharmaceutical composition and wherein said administering occurs once every day. In some embodiments, the amount effective is 3.25 mg of the pharmaceutical composition and wherein said administering occurs once every day. In some embodiments, the amount effective ranges from 1 mg up to and including 10 mg of the pharmaceutical composition and said administering occurs once every day. In some embodiments, the amount effective ranges from 5 mg up to and including 10 mg of the pharmaceutical composition and said administering occurs once every other day. In some embodiments, the amount effective is 10 mg of the pharmaceutical composition and said administering occurs once every other day.

In some embodiments, the pharmaceutically acceptable carrier is chosen from at least one polyethylene glycol compound. In some embodiments, liver fat is reduced and/or maintained by at least 30% up to and including 60%. In some embodiments, the liver fat reduction and/or maintenance is measured as a mean hepatic fat fraction. In some embodiments, said mean hepatic fat fraction is measured using a magnetic resonance imaging derived proton density fat fraction. In some embodiments, said time is for fifty-two weeks. In some embodiments, said time is for thirty-six weeks, one year, two years, five years, ten years, or chronically.

In some embodiments, a method for statistically significantly treating and/or improving fibrosis includes administering an amount effective for said statistically treating and/or improving of a pharmaceutical composition to a human during a time the human is in need of such significant treatment and/or improvement in fibrosis, the composition includes: an amorphous form of VK2809; and a pharmaceutically acceptable carrier.

In some embodiments, the amount effective ranges from 1 mg up to and including 2.5 mg of the pharmaceutical composition and said administering occurs once every day. In some embodiments, the amount effective ranges from greater than 2.5 mg up to and including 4 mg of the pharmaceutical composition and said administering occurs once every day. In some embodiments, the amount effective ranges from 3 mg up to and including 3.5 mg of the pharmaceutical composition and wherein said administering occurs once every day. In some embodiments, the amount effective is 3.25 mg of the pharmaceutical composition and wherein said administering occurs once every day. In some embodiments, the amount effective ranges from 1 mg up to and including 10 mg of the pharmaceutical composition and said administering occurs once every day. In some embodiments, the amount effective ranges from 5 mg up to and including 10 mg of the pharmaceutical composition and said administering occurs once every other day. In some embodiments, the amount effective is 10 mg of the pharmaceutical composition and said administering occurs once every other day.

In some embodiments, the pharmaceutically acceptable carrier is chosen from at least one polyethylene glycol compound. In some embodiments, the treatment and/or improvement to fibrosis is measured using a liver biopsy. In some embodiments, said time is for fifty-two weeks. In some embodiments, said time is for twelve weeks, thirty-six weeks, one year, two years, five years, ten years, or chronically.

In some embodiments, a method for statistically significantly treating and/or improving non-alcoholic fatty liver disease and/or NASH/MASH includes administering an amount effective for said statistically treating and/or improving of a pharmaceutical composition to a human during a time the human is in need of such significant treatment and/or improvement in non-alcoholic fatty liver disease and/or NASH/MASH, the composition includes: an amorphous form of VK2809; and a pharmaceutically acceptable carrier.

In some embodiments, the amount effective ranges from 1 mg up to and including 2.5 mg of the pharmaceutical composition and said administering occurs once every day. In some embodiments, the amount effective ranges from greater than 2.5 mg up to and including 4 mg of the pharmaceutical composition and said administering occurs once every day. In some embodiments, the amount effective ranges from 3 mg up to and including 3.5 mg of the pharmaceutical composition and wherein said administering occurs once every day. In some embodiments, the amount effective is 3.25 mg of the pharmaceutical composition and wherein said administering occurs once every day. In some embodiments, the amount effective ranges from 1 mg up to and including 10 mg of the pharmaceutical composition and said administering occurs once every day. In some embodiments, the amount effective ranges from 5 mg up to and including 10 mg of the pharmaceutical composition and said administering occurs once every other day. In some embodiments, the amount effective is 10 mg of the pharmaceutical composition and said administering occurs once every other day.

In some embodiments, the pharmaceutically acceptable carrier is chosen from at least one polyethylene glycol compound. In some embodiments, the treatment and/or improvement to non-alcoholic fatty liver disease and/or NASH/MASH is measured using a liver biopsy. In some embodiments, said time is for fifty-two weeks. In some embodiments, said time is for twelve weeks, thirty-six weeks, one year, two years, five years, ten years, or chronically.

In some embodiments, a method for reducing and/or maintaining liver fat for longer than a twelve-week time period includes administering a pharmaceutical composition to a human during a time the human is in need of such reduction and/or maintenance in liver fat, the composition includes: a means for reducing and/or maintaining liver fat in said human in need; and a pharmaceutically acceptable carrier.

In some embodiments, said time is for fifty-two weeks. In some embodiments, said time is for thirty-six weeks, one year, two years, five years, ten years, or chronically.

In some embodiments, a method for treating and/or improving fibrosis includes administering a pharmaceutical composition to a human during a time the human is in need of such treatment and/or improvement of fibrosis, the composition includes: a means for treating and/or improving fibrosis in said human in need; and a pharmaceutically acceptable carrier.

In some embodiments, said time is for fifty-two weeks. In some embodiments, said time is for twelve weeks, thirty-six weeks, one year, two years, five years, ten years, or chronically.

In some embodiments, a method for treating and/or improving non-alcoholic fatty liver disease and/or NASH/MASH includes administering a pharmaceutical composition to a human during a time the human is in need of such treatment and/or improvement of non-alcoholic fatty liver disease and/or NASH/MASH, the composition includes: a means for treating and/or improving non-alcoholic fatty liver disease and/or NASH/MASH in said human in need; and a pharmaceutically acceptable carrier.

In some embodiments, said time is for fifty-two weeks. In some embodiments, said time is for twelve weeks, thirty-six weeks, one year, two years, five years, ten years, or chronically.

### BRIEF DESCRIPTION OF THE FIGURES

Aspects of this disclosure are best understood from the following description when read with the accompanying figures.

FIGS. 1A and 1B show graphical representations of mean concentration of a predose of VK2809 over a year (i.e., 52 weeks) measured every 28 days, plotted using a linear and logarithmic ordinate axis, respectively, according to an implementation.

FIGS. 2A and 2B show graphical representations of mean concentration of a predose of VK2809A over a year (i.e., 52 weeks) measured every 28 days, plotted using a linear and logarithmic ordinate axis, respectively, according to an implementation.

FIGS. 3A and 3B show graphical representations of mean plasma concentration of a first dose of VK2809 over 24 hours, plotted using a linear and logarithmic ordinate axis, respectively, according to an implementation.

FIGS. 4A and 4B show graphical representations of mean plasma concentration at 52 weeks after a dose of VK2809 over 24 hours, plotted using a linear and logarithmic ordinate axis, respectively, according to an implementation.

FIGS. 5A and 5B show graphical representations of mean plasma concentration of a first dose of VK2809A over 24 hours, plotted using a linear and logarithmic ordinate axis, respectively, according to an implementation.

FIGS. 6A and 6B show graphical representations of mean plasma concentration at 52 weeks after a dose of VK2809A over 24 hours, plotted using a linear and logarithmic ordinate axis, respectively, according to an implementation.

FIG. 7 shows median relative percentage change in liver fat at 12 weeks after a dose of VK2809A, according to an implementation.

FIG. 8 shows median relative percentage change in liver fat at 12 weeks after a dose of VK2809A, according to an implementation.

FIG. 9 shows median relative percentage change in liver fat at 52 weeks after a dose of VK2809A, according to an implementation.

FIG. 10 shows a representation of percentage change in liver fat in patients with greater than or equal to 30 percent relative reduction in liver fat at 12 weeks after a dose of VK2809A, according to an implementation.

FIG. 11 shows the proportion of patients with greater than or equal to 30 percent relative reduction in liver fat at 52 weeks after a dose of VK2809A, according to an implementation.

FIG. 12 shows a percentage of patients demonstrating resolution of NASH with no worsening of fibrosis at 52 weeks after a dose of VK2809A, according to an implementation.

FIG. 13 shows a percentage of patients demonstrating greater than or equal to 1-stage fibrosis improvement with no worsening of NASH/MASH at 52 weeks after a dose of VK2809A, according to an implementation.

FIG. 14 shows a percentage of patients demonstrating greater than or equal to 1-stage fibrosis improvement with no worsening of NASH/MASH at 52 weeks after a dose of VK2809A, according to an implementation.

FIG. 15 shows a percentage of patients demonstrating greater than or equal to 1-stage fibrosis improvement and resolution of NASH/MASH at 52 weeks after a dose of VK2809A, according to an implementation.

FIG. 16 shows a percentage of patients demonstrating greater than or equal to 1-stage fibrosis improvement and resolution of NASH/MASH at 52 weeks after a dose of VK2809A, according to an implementation.

FIG. 17 shows a change in NIS4 from baseline at 52 weeks after a dose of VK2809A, according to an implementation.

FIG. 18 shows a mean change in lipids at 52 weeks after a dose of VK2809A, according to an implementation.

### DESCRIPTION

VK2809 is a novel TRB-selective agent that uses a proprietary prodrug technology to confer lower systemic and tissue distribution relative to thyroid hormone and other thyromimetic drug classes. Prodrug VK2809 can exist in a mixture of stereoisomers with the cis-isomer being predominant compared to the trans-isomer. The different isomers are shown below.

(2R,4S)-4-(3-chlorophenyl)-2-((4-(4-hydroxy-3-isopropylbenzyl)-3,5-dimethylphenoxy)methyl)-1,3,2-dioxaphosphinane 2-oxide or (2R,4S)-4-(3-chlorophenyl)-2-[[4-(4-hydroxy-3-isopropylbenzyl)-3,5-dimethylphenoxy]methyl]-1,3,2-dioxaphosphinane 2-oxide can be shown by the following structures:

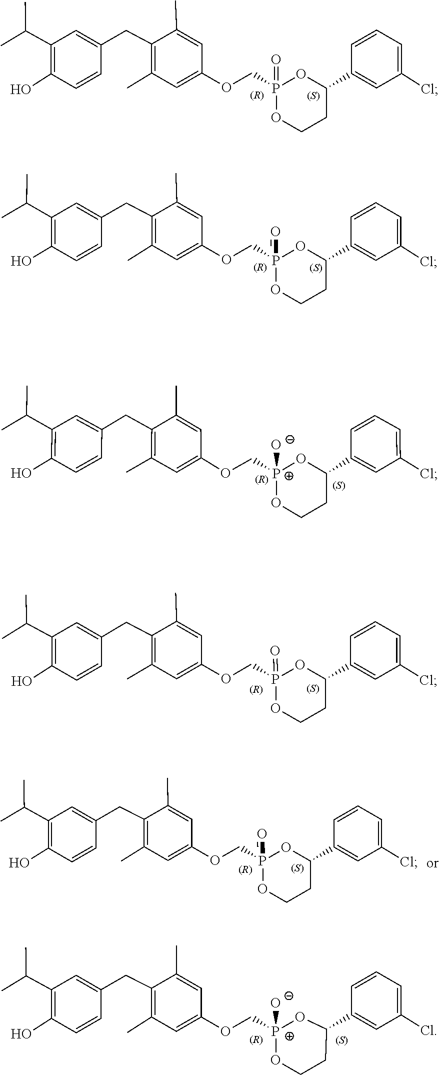

(2R,4R)-4-(3-chlorophenyl)-2-((4-(4-hydroxy-3-isopropylbenzyl)-3,5-dimethylphenoxy)methyl)-1,3,2-dioxaphosphinane 2-oxide or (2R,4R)-4-(3-chlorophenyl)-2-[[4-(4-hydroxy-3-isopropylbenzyl)-3,5-dimethylphenoxy]methyl]-1,3,2-dioxaphosphinane 2-oxide can be shown by the following structures:

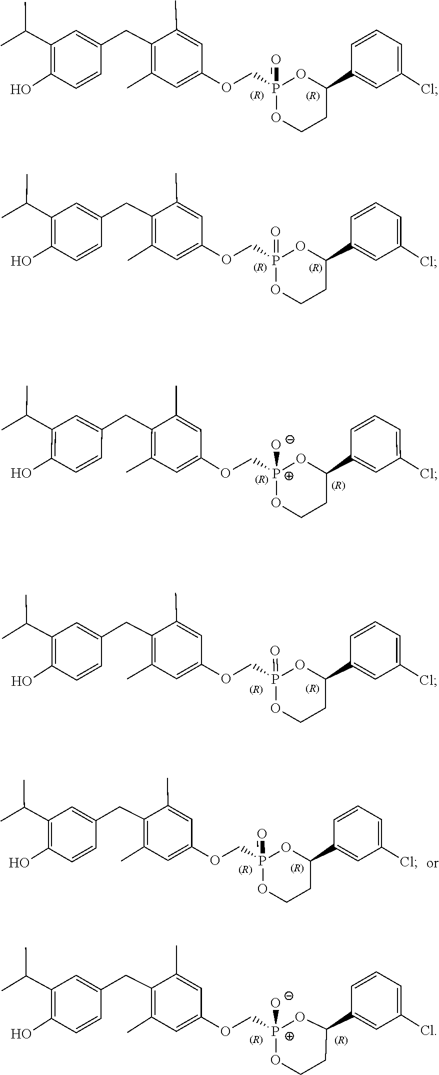

(2S,4S)-4-(3-chlorophenyl)-2-((4-(4-hydroxy-3-isopropylbenzyl)-3,5-dimethylphenoxy)methyl)-1,3,2-dioxaphosphinane 2-oxide or (2S,4S)-4-(3-chlorophenyl)-2-[[4-(4-hydroxy-3-isopropylbenzyl)-3,5-dimethylphenoxy]methyl]-1,3,2-dioxaphosphinane 2-oxide can be shown by the following structures:

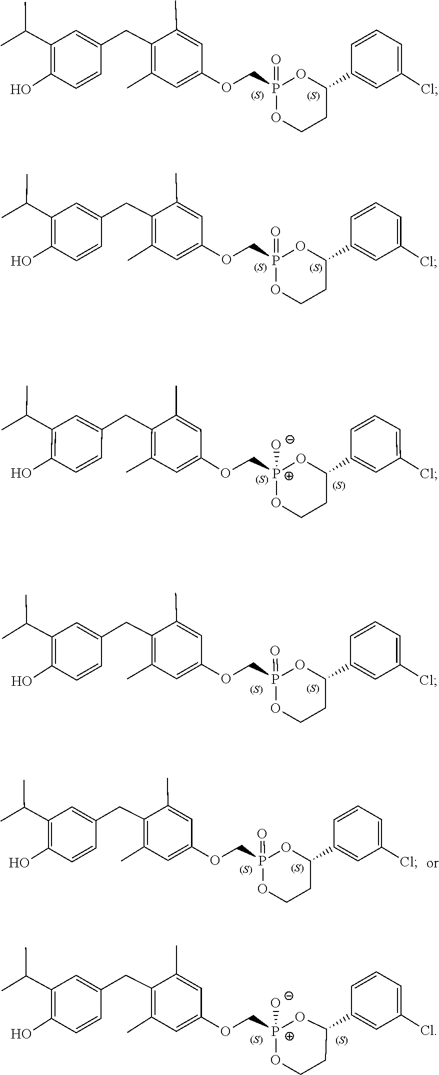

(2S,4R)-4-(3-chlorophenyl)-2-((4-(4-hydroxy-3-isopropylbenzyl)-3,5-dimethylphenoxy)methyl)-1,3,2-dioxaphosphinane 2-oxide or (2S,4R)-4-(3-chlorophenyl)-2-[[4-(4-hydroxy-3-isopropylbenzyl)-3,5-dimethylphenoxy]methyl]-1,3,2-dioxaphosphinane 2-oxide can be shown by the following structures:

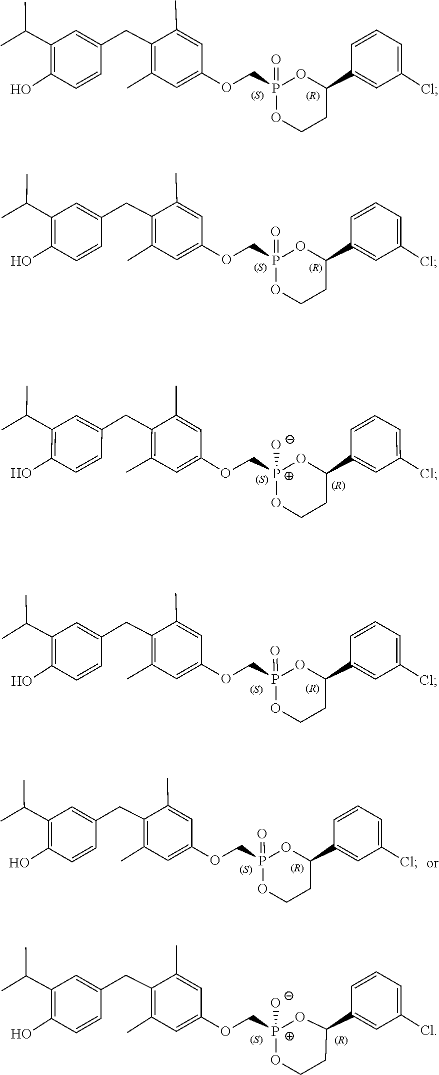

Formula VK2809 is formed of the mixture of the cis- and trans-isomers of VK2809, shown above. The goal of VK2809 treatment is to retain the desired pharmacological effects (e.g., decreased liver fat content and lowered plasma triglycerides) while minimizing any extra-hepatic effects, including cardiovascular toxicity. Following oral dosing, a Cyp3A4-mediated cleavage of prodrug VK2809 occurs. The Cyp3A4 enzyme is primarily expressed in the liver and results in targeted delivery of the VK2809 to the liver. By selective activation, differentiated chemistry lends VK2809 liver selectivity and may minimize the risk of systemic effects.

First-pass extraction of VK2809 in the liver, coupled with liver-specific activation to VK2809A and limited distribution of VK2809A to extra-hepatic tissues, confers a high degree of liver selectivity to the pharmacological effects of this novel agent. Thus, the unique characteristics of VK2809 include the targeted delivery to the liver and the specificity of TRβ-binding activity.

Nonclinical and clinical data indicate a significant benefit of VK2809 in reducing systemic levels of lipids (e.g., low-density lipoprotein cholesterol [LDL-C] and triglycerides) as well as stores of lipids in the liver. Nonclinical data, including pharmacology and toxicology studies, and human clinical efficacy and safety data are summarized below.

As disclosed herein, pharmaceutical compositions including VK2809 in a pharmaceutically acceptable carrier statistically significantly reduces and/or maintains liver fat reduction. The pharmaceutical composition is administered during a time a human is in need of such significant reduction and/or maintenance in liver fat including but not limited to a twelve-week time period, a 36-week time period, a 52-week time period, a 78-week time period, a 5-year time period, a 10-year time period, and/or indefinitely. As such, an effective amount of the pharmaceutical composition may be administered to a human in need of said reduction and/or maintenance in liver fat over a specified time period, intermittently, and/or chronically. Further, the effective amount of the pharmaceutical composition administered may vary or stay constant over said time period based on a human's need of said reduction and/or maintenance.

Further, pharmaceutical compositions including VK2809 in a pharmaceutically acceptable carrier statistically significantly treats and/or improves fibrosis, non-alcoholic fatty disease, and/or NASH/MASH. The pharmaceutical composition is administered during a time a human is in need of such significant treatment and/or improvement of non-alcoholic fatty liver disease and/or NASH/MASH including but not limited to a twelve-week time period, a 36-week time period, a 52-week time period, a 78-week time period, a 5-year time period, a 10-year time period, and/or indefinitely. As such, an effective amount of the pharmaceutical composition may be administered to a human in need of said treatment and/or improvement of non-alcoholic fatty liver disease and/or NASH/MASH over a specified time period, intermittently, and/or chronically. Further, the effective amount of the pharmaceutical composition administered may vary or stay constant over said time period based on the human's need of said treatment and/or improvement.

Details regarding the chemical composition of crystalline VK2809 that is further formulated into tablets and/or capsules as an amorphous solid is now described. Amorphous formulation arises as follows: Excipients (PEG components of formulation) are melted at approximately 60 °C. VK2809 Form C is added to the melt and dissolved with stirring. Upon dissolution, the liquid material is transferred into tablets and/or capsules where the material rapidly solidifies as an amorphous solid. This step happens too quickly and in the presence of other materials to result in Form C. The amorphous material was used in the tablets in the Phase 2b Clinical Study. It was derived from the Form C crystalline material and formulated into tablets as amorphous.

Crystalline forms of VK2809 include tert-butyl methyl ether (TBME) solvate crystalline form, toluene solvate crystalline form, ethanol solvate crystalline form, tetrahydrofuran (THF) solvate crystalline form, ethyl acetate (EtOAc) solvate crystalline form, acetone solvate crystalline form and crystalline Form C. In some embodiments, crystalline Form C can be further melted to form a tablet possessing an amorphous form of VK2809.

The crystalline forms advantageously exhibit improved stability, processability and ease of manufacture. As a result, the crystalline forms of Formula VK2809, particularly crystalline Form C, provide long-term stability and low adsorption and desorption of water vapor.

Crystalline Form C is an unsolvated crystalline form of Formula VK2809. The precise conditions for forming crystalline Form C may be empirically determined and provided herein are exemplary methods found to be suitable in practice.

Crystalline Form C exhibits prominent peaks at approximately 9.1°, 12.4°, 13.8°, 16.0°, 16.6°, 17.1°, 18.6°, 19.1°, 21.6°, 21.7°, and 23.7° two theta (2θ). Thus, a crystalline form of the compounds of Formula VK2809 has at least one characteristic peak (e.g. one, two, three, four, five, six, seven, eight, nine, ten or eleven characteristic peaks) selected from approximately 9.1°, 12.4°, 13.8°, 16.0°, 16.6°, 17.1°, 18.6°, 19.1°, 21.6°, 21.7°, and 23.7° 2θ. The crystalline form of the compounds of Formula VK2809 has at least three characteristic peaks selected from approximately 9.1°, 12.4°, 13.8°, 16.0°, 16.6°, 17.1°, 18.6°, 19.1°, 21.6°, 21.7°, and 23.7° 2θ.

As is well understood in the art, because of the experimental variability when X-ray diffraction patterns are measured on different instruments, the peak positions are assumed to be equal if the 2θ values agree to within a certain degree of variability. For example, the United States Pharmacopeia states that if the angular setting of the 10 strongest diffraction peaks agree to within ±0.2° with that of a reference material, and the relative intensities of the peaks do not vary by more than 20%, the identity is confirmed. Accordingly, in some embodiments, peak positions recited herein include variability within ±0.5° 20. In some embodiments, peak positions recited herein include variability within ±0.2° 20. The term “approximately” when referring to values of 2θ is defined as ±0.5° 2θ.

DSC results for crystalline Form C may indicate a peak at a temperature of about 122 °C, which indicates the melting point for the crystal. Accordingly, crystalline Form C exhibits a melting point from about 117 °C to 127 °C, from about 120 °C to about 124 °C, or at about 122 °C. Crystalline Form C was analyzed by Thermogravimetric analysis (TGA), and in one instance exhibited a 1.3% weight loss and in another instance, exhibited <0.1% weight loss when carried out from 25 °C to 200 °C.

Dynamic vapor sorption (DVS) results for crystalline Form C show a water uptake of less than 0.2% by weight. X-ray power diffraction (XRPD) results following DVA analysis confirm that crystalline Form C did not transition to a different polymorph. The phase equilibration experiment results in water for 3 days for crystalline Form C showed no transformation of the crystalline Form C as seen by XRPD.

Crystalline Form C can therefore be characterized as non-hygroscopic and stable over a wide range of humidity. Crystalline form C also shows good crystallinity, the content of residual solvents is very low (<0.1%), the melting point is relatively high (approx. 172 °C) and crystalline form C does not show any evidence of hydrate formation. In contrast, the solvate forms tend to desolvate producing mixtures of the solvate and the amorphous form. Such advantageous and unexpected non-hygroscopicity and stability demonstrated by crystalline Form C may be capitalized on. For example, in methods of treatment and pharmaceutical compositions, crystalline Form C may provide long-term stability and low adsorption and desorption of water vapor and may provide significant clinical improvements for the treatment of Adrenoleukodystrophy (ALD) a fatty acid buildup disorder and lipid disorders such as hypercholesterolemia and fatty liver diseases.

The compositions, in some embodiments, may include a crystalline form of the compound of Formula VK2809 (e.g., TBMF solvate crystalline form, toluene solvate crystalline form, ethanol solvate crystalline form, THE solvate crystalline form, EtOAc solvate crystalline form, acetone solvate crystalline form, and/or crystalline Form C disclosed herein). In some embodiments, the composition may include at least about 20% by weight of a crystalline form of the compound of Formula VK2809. In some embodiments, the composition may include at least about 50% by weight of a crystalline form of the compound of Formula VK2809. In some embodiments, the composition may include at least about 80% by weight of a crystalline form of the compound of Formula VK2809. In some embodiments, the composition may include at least about 95% by weight of a crystalline form of the compound of Formula VK2809. In some embodiments, the composition may include at least about 50% by weight of crystalline Form C. In some embodiments, the composition may include at least about 80% by weight of crystalline Form C. In some embodiments, the composition may include at least about 95% by weight of crystalline Form C. In some embodiments, the composition may include at least about 99% by weight of crystalline Form C.

The compositions, in some embodiments, include crystalline Form C. For example, the compositions may include at least about 20%, at least about 50%, at least about 90%, at least about 95%, or at least about 99% of crystalline Form C. Similarly, the compositions may also include, for example, the TBME solvate crystalline form, toluene solvate crystalline form, ethanol solvate crystalline form, THE solvate crystalline form, EtOAc solvate crystalline form or acetone solvate crystalline form. The compositions may optionally include at least about 20%, at least about 50%, at least about 90%, at least about 95%, or at least about 99% of the TBME solvate crystalline form, toluene solvate crystalline form, ethanol solvate crystalline form, THF solvate crystalline form, EtOAc solvate crystalline form, acetone solvate crystalline form, and/or crystalline Form C.

The compositions of the compound of Formula VK2809 of the present application may also be formulated for administration to a subject (e.g., a human). The compound of Formula VK2809, and accordingly the compositions disclosed herein, may be formulated for administration with a pharmaceutically acceptable carrier or diluent. The compound of Formula VK2809 may thus be formulated as a medicament with a standard pharmaceutically acceptable carrier(s) and/or excipient(s) as is routine in the pharmaceutical art. The exact nature of the formulation will depend upon several factors including the desired route of administration. Typically, the compound of Formula VK2809 is formulated for oral, intravenous, intragastric, subcutaneous, intravascular or intraperitoneal administration.

The pharmaceutical carrier or diluent may be, for example, water or an isotonic solution, such as 5% dextrose in water or normal saline. Solid oral forms may contain, together with the active compound, diluents, e.g. lactose, dextrose, saccharose, cellulose, corn starch or potato starch; lubricants, e.g. silica, talc, stearic acid, magnesium or calcium stearate, sodium lauryl sulfate, and/or polyethylene glycols; binding agents, e.g. starches, gum arabic, gelatin, microcrystalline cellulose, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, lauryl sulfates; and, in general, non-toxic and pharmacologically inactive substances used in pharmaceutical formulations. Such pharmaceutical preparations may be manufactured in known manners, for example, by means of mixing, granulating, tableting, sugar-coating, or film-coating processes.

Liquid dispersions for oral administration may be syrups, emulsions or suspensions. The syrups may include, as carriers, for example, saccharose or saccharose with glycerine and/or mannitol and/or sorbitol.

Suspensions and emulsions may contain a carrier, for example a natural gum, agar, sodium alginate, pectin, microcrystalline cellulose, methylcellulose, carboxymethylcellulose, or polyvinyl alcohol. The suspensions or solutions for intramuscular injections may contain, together with the compounds of Formula VK2809, a pharmaceutically acceptable carrier, e.g. sterile water, olive oil, ethyl oleate, glycols, e.g. propylene glycol and sodium lauryl sulfate.

The medicament may comprise the compound of Formula VK2809 and a pharmaceutically acceptable carrier.

In some embodiments, the pharmaceutical composition includes the compound of Formula VK2809 in a pharmaceutically acceptable carrier disclosed herein in an effective amount in the range from about 1 mg up to and including about 20 mg. In some embodiments, the pharmaceutical composition has an effective amount in the range from about 1 mg up to about 20 mg. In some embodiments, the pharmaceutical composition has an effective amount in the range from about 1 mg up to 15 mg. In some embodiments, the pharmaceutical composition has an effective amount of about 1 mg, about 2 mg, about 2.5 mg, about 3 mg, about 3.25 mg, about 3.5 mg, about 4 mg, about 5 mg, about 10 mg, or about 15 mg. In some embodiments, the pharmaceutical composition is administered in an amount effective once every day or once every other day.

In some embodiments, an amount effective of the pharmaceutical composition ranges from 1 mg up to and including 2.5 mg administered once every day. In some embodiments, an amount effective of the pharmaceutical composition ranges from 2.5 mg up to and including 5 mg administered once every day. In some embodiments, an amount effective of the pharmaceutical composition ranges from 3 mg up to and including 3.5 mg administered once every day. In some embodiments, an amount effective of the pharmaceutical composition is 3.25 mg administered once every day. In some embodiments, an amount effective of the pharmaceutical composition ranges from 1 mg up to and including 10 mg administered once every day. In some embodiments, an amount effective of the pharmaceutical composition ranges from 5 mg up to and including 10 mg administered once every other day. In some embodiments, an amount effective of the pharmaceutical composition is 10 mg administered once every other day.

Oral formulations may generally include dosages of the compound of Formula VK2809 in the range of about 1 mg to about 100 g. Accordingly, in some embodiments, the oral formulation includes the compound of Formula VK2809 compositions disclosed herein in the range of about 1 mg to about 50 g. In some embodiments, the oral formulation includes the compound of Formula VK2809 compositions disclosed herein in the range of about 1 mg to about 100 mg. In some embodiments, the oral formulation includes the compound of Formula compositions disclosed herein in the range of about 1 mg to about 20 mg. In some embodiments, the oral formulation includes the compound of Formula VK2809 compositions disclosed herein in the range of about 1 mg to about 15 mg. In some embodiments, the oral formulation includes the compound of Formula VK2809 compositions disclosed herein at about 1 mg, about 2 mg, about 2.5 mg, about 3 mg, about 3.25 mg, about 3.5 mg, about 4 mg, about 5 mg, or about 10 mg.

Intravenous formulations may also generally include dosages of the compound of Formula VK2809 in the range of about 1 mg to about 100 g (for example, about 10 mg). In some embodiments, the intravenous formulation has a concentration of about 5 to about 300 mg/mL of the compound of Formula VK2809 (preferably about 25 to about 200 mg/mL, and more preferably about 40 to about 60 mg/mL).

The composition, or medicament including said composition, may optionally be placed in sealed packaging. The sealed packaging may reduce or prevent moisture and/or ambient air from contacting the composition or medicament. In some embodiments, the packaging includes a hermetic seal. In some embodiments, the packaging is sealed under vacuum or with an inert gas (e.g., argon) within the sealed package. Accordingly, the packaging can inhibit or reduce the rate of degradation for the composition or medicament stored within the packaging. Various types of sealed packaging are known in the art.

In some embodiments, Formula VK2809 may be combined with one or more polymers and then further formulated into a desired dosage form. In some embodiments, Formula VK2809 is in the form of a spray dried dispersion (SDD). In other embodiments, Formula VK2809 is in the form of a hot melt extrusion. In some embodiments, the dosage form is an oral dosage form. In some specific embodiments, the oral dosage form is a tablet. In other embodiments, the oral dosage from is a capsule.

In some embodiments, the polymer for preparation of the spray dried dispersion comprising Formula VK2809 may be selected from one or more of polyvinyl pyrrolidone (PVP) polyvinyl pyrrolidone-vinyl acetate copolymer (PVP-VA), polyvinyl alcohol (PVA), polyacrylic acid (PAA), poly(ethylene oxide) (PEO), hydroxypropyl cellulose (HPC), hydroxypropyl methyl cellulose (HPMC), copovidone, poloxamer 407, hypromellose acetate succinate (HPMCAS), Eudragit® polymers, and polyacrylates. In some embodiments, the polymer may be PVP. In some embodiments, the polymer may be PVP-VA. In other embodiments the polymer may be HPMCAS. In yet other embodiments, the polymer may be HPMC. In some embodiments, the polymer may be Eudragit® polymers. In some embodiments, the polymer for the preparation of the spray dried dispersion of Formula VK2809 may be a combination of PVP-VA and poloxamer 407.

Spray dried dispersions of Formula VK2809 with one or more polymers may be prepared by combining Formula VK2809 and polymer in a suitable solvent and then spraying the feed into a hot drying medium to remove the solvent. Preparing a spray dried dispersion (SDD) of Formula VK2809 and one or more polymers may increase the aqueous solubility (and consequently the bioavailability) of Formula VK2809 and may increase the stability of Formula VK2809 such that storage under normal conditions is possible.

In some embodiments, the mass ratio of Formula VK2809 to polymer in the SDD is from about 1:10 to about 10:1. For example, in some embodiments, the mass ratio of Formula VK2809 to polymer in the SDD is about 1:1, 1.5:1, 2:1, 2.5:1, 3:1, 3.5:1, 4:1, 4.5:1 or 5:1. In some embodiments, the mass ratio of Formula VK2809 to polymer in the SDD can be in the range of about 1:1 to 5:1, 1.5:1 to 5:1, 2:1 to 4:1, 2.5:1 to 3.5:1 or 3:1 to 5:1. In some embodiments, the mass ratio of Formula VK2809 to polymer in the SDD can be in the range of about 1:1 to 1:5, 1:1 to 1:4, 1:1 to 1:3, or 1:2 to 1:4. In some embodiments, the mass ratio of Formula VK2809 to polymer in the SDD can be about 1:3.

Formula VK2809 may be formulated into a tablet for oral administration to a subject in need thereof. In some embodiments, Formula VK2809 in the tablet may be present at about 5%, 6%, 7%, 8%, 9%, 10%, 11%, 12%, 13%, 14%, 15%, 16%, 17%, 18%, 19%, 20%, 21%, 22%, 23%, 24%, or 25% by weight of the tablet, or within a range defined by any two of the aforementioned amounts. For example, in some embodiments, Formula VK2809 in the tablet may be present in about 5% to 25%, 10% to 20%, 5% to 15%, or 10% to 15% by weight.

The tablet formulation of Formula VK2809 described herein may further comprise a ductile filler. In some embodiments, the ductile filler in the tablet may be present at about 30%, 35%, 40%, 45%, 50%, 55%, 60%, 65%, or 70% by weight of the tablet, or within a range defined by any two of the aforementioned amounts. For example, in some embodiments, the ductile filler in the tablet may be present in about 30% to 70%, 40% to 60%, 45% to 55%, or 50% to 55% by weight. In some embodiments, the ductile filler may be microcrystalline cellulose. In other embodiments, the ductile filler may comprise silicified microcrystalline cellulose.

The tablet formulation of Formula VK2809 may further comprise a brittle filler. In some embodiments, the brittle filler in the tablet may be present at about 10%, 15%, 20%, 25%, 30%, 35%, or 40% by weight of the tablet, or within a range defined by any two of the aforementioned amounts. For example, in some embodiments, the brittle filler in the tablet may be present in about 10% to 40%, 10% to 30%, 15% to 30%, or 20% to 30% by weight. In some embodiments, the brittle filler may be lactose monohydrate, spray dried lactose, anhydrous lactose, lactose monohydrate, anhydrous lactose, mannitol, or combinations thereof. In some specific embodiments, the brittle filler may be lactose monohydrate. In other specific embodiments, the brittle filler may be mannitol. In yet other specific embodiments, the brittle filler may be anhydrous lactose.

The tablet formulations of Formula VK2809 described herein may further comprise a disintegrant. In some embodiments, the disintegrant in the tablet may be present at about 5%, 6%, 7%, 8%, 9%, 10%, 11%, 12%, 13%, 14%, 15%, 16%, 17%, 18%, 19%, or 20% by weight of the tablet, or within a range defined by any two of the aforementioned amounts. For example, in some embodiments, the disintegrant in the tablet may be present in about 5% to 20%, 5% to 15%, 10% to 15%, or 10% to 20% by weight. In some embodiments, the disintegrant may be carmellose calcium, carboxymethylstarch sodium, croscarmellose sodium, starch, pregelatinized starch, sodium starch glycolate, crosslinked polyvinylpyrrolidone crospovidone, hydroxypropylcellulose, magnesium aluminometasilicate, polacrilin potassium or any combination thereof. In some embodiments, the disintegrant may be croscarmellose sodium. In other embodiments, the disintegrant may be crospovidone.

The tablet formulations of Formula VK2809 described herein may further comprise a glidant. In some embodiments, the glidant in the tablet may be present at about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of the tablet, or within a range defined by any two of the aforementioned amounts. For example, in some embodiments, the glidant in the tablet may be present in about 0.1% to 2.0%, 0.3% to 2.0%, 0.1% to 1.0%, or 0.5% to 1.0% by weight. In some embodiments, the glidant may be silicon dioxide, starch, talc or any combination thereof. In some embodiments, the glidant may be silicon dioxide.

The tablet formulations of Formula VK2809 described herein may further comprise a lubricant. In some embodiments, the lubricant in the tablet may be present at about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of the tablet, or within a range defined by any two of the aforementioned amounts. For example, in some embodiments, the lubricant in the tablet may be present in about 0.1% to 2.0%, 0.3% to 2.0%, 0.1% to 1.0%, or 0.5% to 1.0% by weight. In some embodiments, the lubricant may be stearic acid, talc, glyceryl behenate, sodium stearyl fumarate, magnesium stearate, or any combination thereof. In some specific embodiments, the lubricant may be magnesium stearate.

The tablet formulations of Formula VK2809 described herein may comprise an intragranular component and an extragranular component. For example, in some embodiments, the tablet formulation may comprise an intragranular component comprising Formula VK2809 and one or more polymers; a ductile filler; a brittle filler; a disintegrant; a glidant; and a lubricant; an extragranular component comprising a disintegrant and a lubricant. In some embodiments, the disintegrant in the intragranular component may be the same as the disintegrant in the extragranular component. In other embodiments, the disintegrant in the intragranular component may be different the disintegrant in the extragranular component. In some embodiments, the lubricant in the intragranular component may be the same as the lubricant in the extragranular component. In other embodiments, the lubricant in the intragranular component may be different than the lubricant in the extragranular component.

In some embodiments, the tablet formulations comprising Formula VK2809 described herein comprises about 5%, 6%, 7%, 8%, 9%, 10%, 11%, 12%, 13%, 14%, 15%, 16%, 17%, 18%, 19%, 20%, 21%, 22%, 23%, 24%, or 25% of the one or more polymers, or within a range defined by any two of the aforementioned amounts. For example, in some embodiments, the one or more polymers in the tablet may be present in about 5% to 25%, 10% to 20%, 5% to 15%, or 10% to 15% by weight.

Unlike the non-tablet clinical formulations of Formula VK2809, the tablet formulations comprising Formula VK2809 described herein are stable under standard storage conditions. For example, the tablet formulations described herein show minimal degradation as measured by HPLC upon storage in high density polyethylene bottles for one, two, and three months at 40 °C or 50 °C and ambient relative humidity (RH). In some embodiments, the amount of Formula VK2809 in the tablet formulation may comprise 95%, 96%, 97%, 98%, 99% or more by weight of the original amount of Formula VK2809 in the formulation upon storage in a closed bottle for 1 month at 40 °C and ambient RH. In some embodiments, the amount of Formula VK2809 in the tablet formulation may comprise 95%, 96%, 97%, 98%, 99% or more by weight of the original amount of Formula VK2809 in the formulation upon storage in a closed bottle for 1 month at 50 °C and ambient RH. In some embodiments, the amount of Formula VK2809 in the tablet formulation may comprise 95%, 96%, 97%, 98%, 99% or more by weight of the original amount of Formula VK2809 in the formulation upon storage in a closed bottle for 2 months at 40 °C and ambient RH. In some embodiments, the amount of Formula VK2809 in the tablet formulation may comprise 95%, 96%, 97%, 98%, 99% or more by weight of the original amount of Formula VK2809 in the formulation upon storage in a closed bottle for 2 months at 50 °C and ambient RH. In some embodiments, the amount of Formula VK2809 in the tablet formulation may comprise 95%, 96%, 97%, 98%, 99% or more by weight of the original amount of Formula VK2809 in the formulation upon storage in a closed bottle for 3 months at 40 °C and ambient RH. In some embodiments, the amount of Formula VK2809 in the tablet formulation may comprise 95%, 96%, 97%, 98%, 99% or more by weight of the original amount of Formula VK2809 in the formulation upon storage in a closed bottle for 3 months at 50 °C and ambient RH.

Tablets were prepared by combining all intragranular materials (i.e., the active intermediate, ductile filler, brittle filler, disintegrant and glidant) except lubricant and blending the material in a 16 quart V-shell blender for 60 revolutions at a speed of 16 revolutions per minute (rpm) to form a pre-blend. The pre-blend was then delumped by blending through a Quadro Comil Model U5 using a 032R screen using a round mill impellor at a speed of 1500+/−100 rpm. The delumped material was then returned to the 16 quart V-shell blender and blended for 180 revolutions at 16 rpm to form a pre-granulation main blend. The pregranulation blends prepared according to this example had high bulk and tapped densities, and a relatively low Carr index, indicating that the pregranulation blends should have acceptable flow characteristics for roller compaction. The pregranulation blends were also found to be highly compressible, tabletable, and compactable.

The intragranular lubricant was added to the pre-granulation main blend, mixed manually for about 15-30 seconds and then passed through a #20 mesh screen back into the blender and mixed for 48 revolutions at 16 rpm to form the main lubricant pregranulation blend. The main lubricant pregranulation blend was roller compacted using a Gerties Mini-Pactor using the settings in Table 1 to form an intragranular blend.

TABLE 1

| | |
|---|---|
| Parameter | Settings |

Roll Settings

| | |
|---|---|
| Master Roll (lower, left position) | Knurled |
| Second Roll (upper, right position) | Knurled |
| Compression Force (kN/cm) | 3.0 |
| Press Roller Start Speed (rpm) | 0.5 |
| Press Roller Speed (rpm) | 1.5 |
| Gap Width (mm) | 2.0 |
| PID (unitless:msec:msec) | 3:8000:0 |

Feed Settings

| | |
|---|---|
| Agitator Speed (rpm) | 5 |
| Feed Factor | 0.7 |
| Density Factor | 0.7 |
| Tamp/Feed Ratio | 180 |
| Gap-Control Activated (On/Off) | On |
| Torque Control (On/Off) | Off |

Granulator Settings

| | |
|---|---|
| Granulator Type | Pocket |
| Granulator Screen Type | Square- |
| Granulator Screen Size (mm) | 1.00 |
| Granulator Rotor Block Setting (mm) | 1.0 |
| Granulator Speed (rpm) | 50 |
| Granulator CW/CCW Degreesa, b | 240/360 |

a CW = clockwise rotation; CCW = counter-clockwise rotation.

Extragranular disintegrant was added to the intragranular blend and blended in an 8 quart V-shell blender for 180 revolutions at a speed of 18 rpm to form an extragranular main blend. Extragranular lubricant was passed through a #20-mesh screen and added to the extragranular main blend and blended from 48 revolutions at 18 rpm to form an extragranular lubricant blend. The extragranular blends prepared according to this example had high bulk and tapped densities, and a relatively low Carr index, indicating that the pregranulation blends should have acceptable flow characteristics for roller compaction.

The extragranular lubricant blend was then compressed on a Korsch XL100 tablet press ( 9/32″ SRC tooling, Sotax ST50 tablet hardness tester) to form tablets with a target weight of 150 mg. The tablet press was adjusted to achieve target tablet weight and hardness as needed.

The tablets were then dedusted with a vibratory and/or vacuum deduster. The compression parameters are provided in Table 2 below.

TABLE 2

| | |
|---|---|
| Paramete | Setting |

Tablet Press

| | |
|---|---|
| Tooling Size and Shape | 9/32″ SRC |
| Tooling drawing number | 91270 |
| Number of tooling stations | 5 |
| Turret Speed (rpm) | 20 |
| Fill Cam (mm) | 4-10 |
| Feed Frame Paddle Type | Standard |
| Dosing Depth | 5.90 |
| Pre-Compression Insertion Depth (mm) | Adjust as needed |
| Main Compression Insertion Depth (mm) | Adjust as needed |
| Main Compression Edge Thickness (mm) | 1.75 |
| Pre-Compression Force (kN) | 0.2 |
| Main Compression Force (kN) | 4.4 |
| De-duster | Yes - Post |

Bulk Tablet Attribute Averages (n = 50)

| | |
|---|---|
| Weight (mg) | 150.4 |
| Tablet Thickness (mm) | 3.9 |
| Tablet Hardness (kp) | 7.62 |

Table 3 below is a list of abbreviations used herein.

TABLE 3

| Abbreviation | Definition |
|---|---|
| AE | adverse event |
| ALP | alkaline phosphatase |
| ALT | alanine transaminase |
| ANCOVA | analysis of covariance |
| AST | aspartate transaminase |
| BMI | body mass index |
| BNP | B-type natriuretic peptide |
| BPM | beats per min |
| CRN | Clinical Research Network |
| CRP | C-reactive protein |
| DMC | Data Monitoring Committee |
| ECG | electrocardiogram |
| eCRF | electronic case report form |
| FAS | Full Analysis Set |
| GLP-1 | glucagon-like peptide-1 |
| HCV | hepatitis C virus |
| HEV | hepatitis E virus |
| ICF | informed consent form |
| IgM | immunoglobulin M |
| IRB | Institutional Review Board |
| LDL-C | low-density lipoprotein cholesterol |
| MASH | Metabolic dysfunction associated steatohepatitis |
| MRI-PDFF | magnetic resonance imaging-proton density fat fraction |
| NAFLD | non-alcoholic fatty liver disease |
| NAS | NASH activity score |
| NASH | non-alcoholic steatohepatitis |
| NIS4 | non-invasive score-4 |
| P111NP | procollagen N-terminal propeptide |
| PK | pharmacokinetics |
| PPS | per protocol set |
| PT | preferred term |
| QD | once daily |
| QOD | once every other day |
| QT | interval between the beginning of the QRS complex and the |
| | end of the T-wave |
| QTc | corrected QT |
| QTcB | QT interval corrected with Bazett's formula |
| QTcF | QT interval corrected with Fridericia's formula |
| SOP | standard operating procedure |
| T3 | triiodothyronine |
| T4 | thyroxine |
| TEAE | treatment emergent adverse event |
| TIMP1 | tissue inhibitor of metalloproteinases |
| TSH | thyroid stimulating hormone |
| ULN | upper limit of normal |

Table 4 below shows a linking table.

TABLE 4

| Recited Function | Exemplified Materials |
|---|---|
| “a means for reducing and/or maintaining | Form(s) of VK2809 discussed in ¶¶[0045]- |
| liver fat in said human in need” | [0050], [0055]-[0065] and administered in an |
| | effective amount discussed in ¶¶[0071]- |
| | [0074] |
| “a means for treating and/or improving | Form(s) of VK2809 discussed in ¶¶[0045]- |
| fibrosis in said human in need” | [0050], [0055]-[0065] and administered in an |
| | effective amount discussed in ¶¶[0071]- |
| | [0074] |
| “a means for treating and/or improving non- | Form(s) of VK2809 discussed in ¶¶[0045]- |
| alcoholic fatty liver disease and/or | [0050], [0055]-[0065] and administered in an |
| NASH/MASH in said human in need” | effective amount discussed in ¶¶[0071]- |
| | [0074] |
| “a means for treating and/or improving | Form(s) of VK2809 discussed in ¶¶[0045]- |
| fibrosis, non-alcoholic fatty liver disease, | [0050], [0055]-[0065] and administered in an |
| and/or NASH/MASH in said human in need” | effective amount discussed in ¶¶[0071]- |
| | [0074] |
| “a pharmaceutically acceptable carrier” | Acceptable carriers discussed in ¶¶[0066]- |
| | [0092] |

### Examples

The goal of this Phase 2B Clinical Study was to evaluate the efficacy and safety of VK2809 administered to participants with NASH/MASH. In the previous study (Phase 2A Clinical Study), VK2809 was highly effective in treating participants with NAFLD, as measured by liver fat reduction. The study examined the effects of 10.0 mg every day (QD), 10.0 mg every other day (QOD), and 5.0 mg everyday (QD) treatment for 12 weeks.

The Phase 2B Clinical Study examined additional doses 10.0 mg QOD, 5.0 mg QOD, 2.5 mg QD, and 1.0 mg QD, for 52 weeks. The degree of fibrosis and NASH/MASH activity score (NAS) were determined with histology measurements.

The primary objective was to evaluate the effects of VK2809 compared to placebo on liver fat content. The secondary objectives were to evaluate the effects of VK2809 compared to placebo on histologic changes assessed by hepatic biopsy and to assess the safety and tolerability of VK2809 administered for 52 weeks.

The Phase 2B Clinical Study was a multicenter study that included 3 periods: a screening period (up to 12 weeks), a treatment period (52 weeks), and a follow-up period (4 weeks). Participants were randomized to 1 of 5 treatment arms: VK2809 1.0 mg QD, 2.5 mg QD, 5.0 mg QOD, 10.0 mg QOD, or placebo QD.

Screening procedures were performed over multiple study visits within 84 days (12 weeks). The participant screening period was extended to allow for confirmation of eligibility if the biopsy sample was collected within the required 84 day screening period, if central reading of liver biopsy was pending.

Participants who met entry criteria were randomized at visit 2. The treatment period consisted of a 52-week, double-blind treatment period with clinic visits from randomization and every 4 weeks from Week 4 to Week 56.

After completion of the treatment period, participants entered a 4-week, off-drug follow-up period. The end of study visit (EOS) occurred at Week 56. The follow-up period included all participants, including those who had discontinued treatment with study drug during the treatment period.

Doses included in the Phase 2B Clinical Study were based on previous clinical studies, which demonstrated VK2809 at the doses used in this Phase 2B Clinical Study were safe and well tolerated. The placebo arm as a comparator to VK2809 arms was included to fully assess safety and efficacy and to comply with FDA guidance.

Participants who met the following key inclusion criteria were included in the study:
Have a histologically confirmed diagnosis of NASH/MASH on a liver biopsy performed during screening or within 6 months before screening;
Have a screening MRI PDFF with ≥8% liver fat fraction; or
Male and females were 18 to 75 years of age, inclusive, at screening.

Participants who met any of the following key exclusion criteria were excluded from the study:
Were unwilling to undergo the required liver biopsy procedures or have any condition that would prevent obtaining a liver biopsy as part of the clinical protocol;
Have evidence of current or history of excessive alcohol consumption of more than 20 g per day for women and 30 g per day for men, on average, within 6 months before the qualifying liver biopsy and up to randomization or were unable to provide a reliable estimate of alcohol consumption during that period;
Have evidence (positive drug and alcohol test) of or acknowledged use of illicit drugs and excessive alcohol consumption within 6 months before screening;
Have had weight change >5% within 3 months before the qualifying liver biopsy and up to randomization;
Treatment with medications for the purpose of weight loss within 6 months before qualifying liver biopsy, unless approved after consultation with the medical monitor. These included drugs approved for weight loss (eg, orlistat, bupropion/naltrexone, phentermine-topiramate, phentermine, lorcaserin) as well as drugs used off-label, herbal preparations, and dietary supplements marketed for control of body weight or appetite;
History of eating disorders;
Active involvement in a structured weight loss program (including programs such as Weight Watchers, Jenny Craig, etc.) within 6 months before qualifying liver biopsy, unless approved after consultation with the medical monitor;
Have a history of:
   type 1 diabetes or latent autoimmune diabetes in adults;
   uncontrolled type 2 diabetes (hemoglobin Alc≥9% at screening);
Have a history of:
   solid organ or bone marrow transplantation;
   active hyperthyroidism of any cause, active thyroid malignancy, or other active uncontrolled thyroid disorder;
Have a history of effective bariatric surgery before qualifying for the study (participants with ineffective surgery could participate after consultation with the medical monitor);
Have a diagnosis (or current evidence) of other chronic liver diseases;
Have any active serious medical disease with likely life expectancy ≤5 years;
Have any major cardiovascular events (myocardial infarction, unstable angina, coronary revascularization, stroke, transient ischemic attach) within 6 months before screening;
Congestive heart failure (New York Heart Association Class 2 to 4), greater than Class 1 angina pectoris, acute coronary syndrome in the last 6 months, peripheral vascular disease that limits ambulation, uncontrolled hypertension (systolic blood pressure [SBP]>150, diastolic blood pressure [DBP]>90) at screening, known structural heart disease with left ventricular ejection fraction <40%, ventricular tachycardia, atrial fibrillation requiring anticoagulation, documented ongoing paroxysmal atrial fibrillation, or without adequate heart rate control (mean heart rate >100 beats per minute [BPM]), implantable cardioverter-defibrillator implantation, history in the last 3 years of symptomatic paroxysmal supraventricular tachycardia, personal or family history of sudden death or long QT syndrome in the last 6 years, unexplained syncope or syncope within the last 2 years regardless of etiology.
Electrocardiogram (ECG):
   QT interval corrected with Fridericia's formula (QTcF)>450 msec;
   PR interval >240 msec;
   heart rate >100 BPM;
Screening Holter including nonsustained ventricular tachycardia (at least 3 ventricular beats at a heart rate >100 BPM), supraventricular tachycardia ≥15 beats in duration at a rate >100 BPM, mean atrial fibrillation ventricular rate >100 BPM, paroxysmal atrial fibrillation.

Participants who met any of the following key exclusion criteria based on laboratory screening results were excluded from the study:
Thyroid stimulating hormone outside central laboratory reference range;
Free thyroxine <0.6 ng/dL or >1.8 ng/dL;
Cardiac troponin I and creatine kinase-MB isoenzyme >ULN at screening;
Serum albumin <3.5 g/dL;
International normalized ratio >1.3 unless due to anticoagulant therapy;
Total bilirubin >1.2×ULN (except in presence of Gilbert syndrome);
Direct bilirubin >ULN;
Platelet count <140,000/mm3;
Eosinophil count (absolute)>1,000 cells/mm3 (>1,000/μL);
ALT >5×ULN;
AST >5×ULN; or
Estimated glomerular filtration rate <60 ml/min/1.73m2 (by the CKD-Epidemiology Collaboration Method).

Participants who satisfied all entry criteria were randomized to receive 1 of 5 treatments with equal probability with the exception of the 1.0 mg QD and 5.0 mg QOD VK2809 arms. All study drug doses were administered orally. The dosing arms included:
VK2809 1.0 mg QD;
VK2809 2.5 mg QD;
VK2809 5.0 mg QOD;
VK2809 10.0 mg QOD;
Placebo QD.

All participants received 1 capsule daily to maintain blinding. Participants in QOD arms received blister packs designed for alternate day dosing containing both VK2809 and placebo capsules.

Upon implementation of Amendment 8, participants were no longer randomized to the 1.0 mg QD arm. An interim analysis was performed on participants who completed an MRI at Week 12. Upon completion of the interim analysis, new participants were enrolled in the placebo, 10.0 mg QD, and the 2.5 mg QD arms. Upon implementation of Amendment 10, participants were not randomized to the 5.0 mg QOD arm.

VK2809 was supplied in a capsule formulation containing VK2809 1.0 mg, VK2809 2.5 mg, VK2809 5.0 mg, or VK2809 10.0 mg. Placebo was supplied in a matching capsule.

To date, approximately 200 participants have received VK2809 in clinical trials. Of these, 91 participants had hyperlipidemia, including 42 with NAFLD. VK2809 has been evaluated at doses ranging from 0.25 mg to 50 mg and has been administered for durations as long as 12 weeks.

Trials conducted with VK2809 in participants with hyperlipidemia, including participants with NAFLD, have shown consistent reductions in circulating lipids, particularly total cholesterol, LDL-C, triglycerides, nonhigh-density lipoprotein cholesterol (nonHDL-C), Apo-B and lipoprotein (a) (Lp(a)). In addition, liver fat was significantly reduced by more than 50% in participants with NAFLD. The AE profiles of participants who received VK2809 and those who received placebo in these trials have been quite similar. Mild increases above reference ranges were observed in ALT and AST (lower magnitude than ALT) that appeared to be dose dependent. The elevations generally resolved during the study without dose interruption. No evidence of cardiovascular toxicity or derangements in thyroid function (hypothyroidism or thyrotoxicosis) has been observed in participants receiving VK2809.

Each participant received either VK2809 1.0 mg QD, 2.5 mg QD, 5.0 mg QOD, 10.0 mg QOD, or placebo QD according to the randomized treatment assignment. During the double-blind treatment period, the investigational product was taken orally at the appropriate time in the morning after 8 hours or more of fasting for dosing days. Participants were instructed to fast (water allowed) for 30 minutes after the dose of double-blind investigational product.

AEs were collected from the time of informed consent to completion of the follow-up period (Visit 16, Week 56). All SAEs, AEs leading to discontinuation, and AEs of special interest were followed to resolution. The severity of each AE was graded according to the National Cancer Institute Common Terminology Criteria for Adverse Events (CTCAE) version 5.0, as follows:
Grade 1-Mild asymptomatic or mild symptoms; clinical or diagnostic observations only; intervention not indicated;
Grade 2-Moderate, minimal, local or non-invasive intervention indicated; limiting age-appropriate instrumental activities of daily living (ADL) (preparing meals, shopping for groceries or clothes, telephone use, managing money, etc.);
Grade 3-Severe or medically significant but not immediately life-threatening; hospitalization or prolongation of hospitalization indicated; disabling; limiting self-care ADL (bathing, dressing, undressing, feeding self, toileting, taking medications, and not bedridden);
Grade 4-Life-threatening consequences; urgent intervention indicated; or
Grade 5-Death related to an AE.

Actions taken with the study drug, other treatment measures for the AE, and outcome of the AE were recorded.

Pre- and postdose Sparse PK samples were collected from all participants at every clinic visit except for screening and EOS. A subset of participants had intensive PK assessments completed as follows:
6 participants in each QD arm (1.0 mg and 2.5 mg) and at each fibrosis stage (F1-F3) [36 participants total];
6 participants in each QOD dose arm (5.0 mg and 10.0 mg) and at each fibrosis stage (F1-F3) [36 participants total];
Placebo: 8 participants at each fibrosis stage (F1-F3) [24 participants total].

Samples were collected at the following time points:
Day 1: predose, postdose at 1, 2, 4, 8 and 24 hours;
Week 52: predose, postdose at 1, 2, 4, 8 and 24 hours (predose was used as the 48-hour time point for area under the curve [AUC] calculations for the QOD dosing arms). PK samples were obtained from whole blood predose and at designated times postdose starting at Day 1. Plasma PK parameters may have included but were not limited to: Cmax, AUClast, and tmax.

The primary efficacy endpoint was the relative change in liver fat content (assessed by MRI-PDFF) from baseline to Week 12 in participants treated with VK2809 versus placebo. The following secondary efficacy endpoints compared changes in participants treated with VK2809 versus placebo:
Absolute change in liver fat content (assessed by MRI-PDFF) from baseline to Week 52;
Relative change in liver fat content (assessed by MRI-PDFF) from baseline to Week 52;
Proportion of participants with a relative percent decrease in liver fat content ≥30% (assessed by MRI-PDFF) from baseline to Week 12 and Week 52.

The following secondary efficacy endpoints compared central reader scored histopathologic assessments (liver biopsies) from baseline to Week 52 for participants treated with VK2809 versus placebo:
Proportion of participants with resolution of steatohepatitis on overall histopathological reading and no worsening of liver fibrosis on NASH Clinical Research Network (CRN) fibrosis score. Resolution of steatohepatitis was defined as absent fatty liver disease or isolated or simple steatosis without steatohepatitis and a NAS score (non-alcoholic steatohepatitis activity score) of 0 to 1 for inflammation, 0 for ballooning, and any value for steatosis;
Proportion of participants with improvement in liver fibrosis greater than or equal to one stage (NASH CRN fibrosis score) and no worsening of steatohepatitis (defined as no increase in NAS for ballooning, inflammation, or steatosis);
Proportion of participants with >2-point improvement in NAS (with at least 1-point improvement in ballooning or inflammation) with no worsening of fibrosis stage;
Proportion of participants with resolution of steatohepatitis and improvement in fibrosis.

The following exploratory endpoints compared responses from baseline to Week 12 and Week 52 for participants treated with VK2809 versus placebo:
Proportion of participants with normalization of liver fat (assessed by MRI-PDFF and defined as a fat fraction of <5%);
Proportion of participants with an absolute reduction ≥5% in liver fat content (assessed by MRI-PDFF);
Assessment of the presence or absence of NASH/MASH on paired biopsy review;
Assessment of overall NASH/MASH as better, same, or worse on paired biopsy review;
Assessment of NASH/MASH subcomponents as better, same, or worse on paired biopsy review;
Assessment of fibrosis as better, same, or worse on paired biopsy review;
Changes in liver enzymes (e.g., ALT, AST, alkaline phosphatase, gamma-glutamyl transferase);
Changes in fasting lipid levels (e.g., LDL-C, total cholesterol, high-density lipoprotein cholesterol, triglycerides, apolipoprotein B, Lp (a), apolipoprotein C-III);
Changes in inflammation biomarkers (high sensitivity C-reactive protein);
Changes in markers of hepatic fibrosis and steatosis (e.g., enhanced liver fibrosis score, non-invasive score-4 [NIS4], procollagen N-terminal propeptide (P111NP), tissue inhibitor of metalloproteinases 1 [TIMP1])/creatinine);
Assessment of genetic polymorphisms potentially associated with increased risk of NASH/MASH and increased risk of fibrosis (including genetic material sequencing). Genetic testing was optional, and participants could decline to provide a sample;
Changes in corrected T1;
Change from baseline in Ishak fibrosis score;
Proportion of participants with improved Ishak fibrosis score;
Proportion of participants with worsening Ishak fibrosis score;
Proportion of participants with stable Ishak fibrosis score;
Percentage of participants with at least 1-point improvement in Ishak fibrosis score;
Assessment of fibrosis and steatosis with dual-photon microscopy in unstained tissue;
Assessment of sparse and intensive plasma PK (pre- and postdose levels) for participants that choose to complete.

The safety and tolerability of VK2809 was assessed by evaluation of:
AEs;
Vital signs;
Physical examinations;
Safety laboratories, including hematology, blood chemistry, liver, thyroid function tests, and cardiac biomarkers tests (cardiac troponin I, N-terminal pro B-type natriuretic peptide, creatine kinase, and creatine kinase-MB) urinalysis;
Liver laboratories (ALT, AST, alkaline phosphatase, bilirubin [total and direct], gamma-glutamyl transferase, prothrombin time, and international normalized ratio, and albumin);
Cardiac safety monitoring, including resting 12-lead ECGs and Holter monitoring;
Changes in bone markers (provided by the central laboratory);
Diabetic management, including incidence of hypoglycemia and hyperglycemia.

Blood samples (approximately 5 mL) were collected for Sparse PK to determine plasma concentrations of VK2809 and VK2809A for all participants predose at the following visits: visit 2 (randomization), visit 3 (Week 4), visit 4 (Week 8), visit 5 (Week 12), visit 6 (Week 16), visit 9 (Week 28), visit 12 (Week 40), and visit 15 (Week 52). At visit 2 and visit 15, blood samples were collected from the intensive PK subset at predose and postdose at 1 hour, 2 hours, 4 hours, 8 hours, and 24 hours.

Table 5 below shows the analysis populations described above:

TABLE 5

| Population | Description |
|---|---|
| Safety | The Safety Population included all participants treated with |
| Population | at least 1 dose of study drug. |
| Full Analysis | The FAS consisted of all randomized participants who had |
| Set (FAS) | at least 1 dose of study drug and had at least 1 postbaseline |
| | MRI-PDFF assessment. |
| Per Protocol | The PPS consisted of all participants in the FAS who did |
| Set (PPS) | not have any important protocol deviations leading to |
| | exclusion from the PPS and had evaluable MRI-PDFF data |
| | at baseline and Week 12. |
| Pharma- | The PK Analysis Set included the subset of participants in |
| cokinetic | the Safety Population who had at least 1 blood sample |
| (PK) Set | collected for PK sampling and had sufficient assay results |
| | to permit the calculation of at least 1 PK parameter. |

General statistical methods included Version 9.4 (or later) of the SAS® statistical software package was used to provide summaries, listings, figures, PK, and statistical tables and analyses. All measured variables and derived parameters were listed individually and, if appropriate, tabulated by descriptive statistics. Participants were analyzed according to the drug received. Summary tables for descriptive statistics included number of participants, mean, standard deviation, median, minimum, and maximum. Summary tables for categorical variables included by study arm summaries of frequency counts and percentages. Table 6 below shows the study population analysis methods:

TABLE 6

| Endpoint | Methods |
|---|---|
| Participant | Participant disposition for all screened participants were |
| Disposition | listed and summarized by treatment arm and overall |
| and Data | (total) and included the number and percentage of |
| Sets | participants: |
| Analyzed | screened |
| (SAP | randomized |
| Section 7.2) | randomized and not treated |
| | in each study population |
| | The number and percentage of participants who |
| | completed the study and who discontinued early were |
| | presented. |
| | A summary of participant enrollment by country and site |
| | was provided (by treatment arm and overall) for the FAS |
| | population. |
| | A summary of screen failures and number of participants |
| | screened but not randomized was produced. |
| Protocol | Protocol deviations were listed by participant and |
| Deviations | treatment arm for the FAS population. Important protocol |
| (SAP | deviations leading to exclusion from the PP population |
| Section 7.3) | were summarized by treatment arm and listed by |
| | participants and treatment arm for the FAS population. |
| | Important deviations included prohibited medications, |
| | treatment compliance (<80% to >120% limits on |
| | cumulative dose), entry criteria violations, missing/out of |
| | window efficacy assessments, randomization |
| | misallocation, missed visits or visit compliance, or |
| | unblinding. |
| Demographics | Demographic and baseline characteristics were listed and |
| and Baseline | summarized by treatment arm and overall for all analysis |
| Characteristics | populations. Standard descriptive statistics were |
| (SAP | presented for the continuous variables of age; baseline |
| Section 7.4) | weight; height; body mass index; and baseline ALT, |
| | AST, NAS score, liver fat content, and ECG parameters. |
| | The total counts and percentages of participants were |
| | presented for the categorical variables of age group |
| | (years); sex; race; ethnicity; childbearing potential |
| | of female participants and method of contraception or |
| | reason for sterilization; type II diabetes status; and |
| | NASH CRN fibrosis stage (F1, F2, F3). |
| | Vital signs and laboratory values were summarized by |
| | treatment arm with postbaseline measurements. |
| Medical | Medical history was coded using the Medical Dictionary |
| History | for Regulatory Activities version 22.1 or later. |
| (SAP | All medical history was listed, and the number and |
| Section 7.4.1) | percentage of participants with any medical history were |
| | summarized for the Safety Population by system organ |
| | class and preferred term for each treatment arm and |
| | overall. |
| Previous and | Previous or concomitantly were mapped to the World |
| Concomitant | Health Organization Drug Dictionary (version September |
| Medications | 2019 Enhanced B3 or later) and Anatomical Therapeutic |
| (SAP | Chemical (ATC) Classification codes. |
| Section 7.4.2) | Prior medications and concomitant medications were |
| | listed together and summarized separately for the safety |
| | analysis population. |
| | The number and percentage of participants taking each |
| | medication were displayed together with the number and |
| | percentage of participants using at least one medication |
| | within each therapeutic class (ATC-Level 2), chemical |
| | subgroup (ATC-Level 4), and generic term. |
| Measurements | Percentage compliance was summarized descriptively by |
| of Treatment | treatment arm for the Safety Population. |
| Compliance | The number and percentage of compliant participants |
| (SAP | were presented for the Safety Population, including |
| Section 7.5) | compliance categories of <80% and >120%. |

All PK analyses were performed on the PK population unless otherwise noted. PK samples were analyzed for VK2809 and VK2809A according to a validated bioanalytical method. Table 7 below shows the pharmacokinetic analysis methods:

TABLE 7

| Endpoint | Methods |
|---|---|
| PK Analysis | Plasma PK parameters for VK2809 and VK2809A for |
| (SAP | participants with intensive PK samples collected on Day |
| Section | 1 and Week 52 were determined with noncompartmental |
| 7.8.1) | methods and Phoenix WinNonlin (Certara, v8.1 or higher) |
| | and included: |
| | AUCₗₐₛₜ |
| | AUC_{0-24 h} |
| | AUC_{0-48 h} |
| | Cₘₐₓ |
| | Tₘₐₓ |
| | Tₗₐₛₜ |
| | AR_{AUC} |
| | MP_{AUC} |
| | MPCₘₐₓ |
| | Additional PK parameters were determined where data |
| | allowed |
| PK Data | Participants with insufficient sample size that prevented the |
| Presentation | calculation of PK parameters had individual plasma |
| (SAP | concentrations included in the participant listings, but |
| Section | they were not included in the PK parameter summaries. |
| 7.8.2) | Summary tables, arithmetic mean (+standard deviation |
| | [SD]) figures, overlaying individual figures, and |
| | individual figures by treatment and time postdose were |
| | provided for plasma PK concentrations. Linear and |
| | semilogarithmic scales PK concentration figures were |
| | produced. |
| | PK parameters were summarized with descriptive statistics |
| | including the arithmetic and geometric means, median, 25^{th} |
| | and 75^{th} percentiles, SD, coefficient of variation, and |
| | minimum and maximum values. Excluded participants were |
| | listed but were excluded from the descriptive statistics. |

All safety analyses were performed on the Safety Population. Participants were summarized by arm, treatment visit, and time. Table 8 below shows the safety analysis methods:

TABLE 8

| Endpoint | Methods |
|---|---|
| Extent of Exposure | Exposure to study drug was summarized with mean, SD, |
| (SAP Section 7.7.1) | median, minimum, and maximum for the duration of |
| | exposure (days), duration of interruption (days), and total |
| | cumulative dose (mg). |
| | The reason for interruption (AE or other) was summarized |
| | with the frequency and percentage of participants by |
| | treatment arm. |
| Adverse Events | AEs were coded with the MedDRA dictionary v22.1 (or |
| (SAP Section 7.7.2) | later). AE severity was graded with CTCAE version 5.0. |
| | The number and percentage of participants reporting each AE |
| | and number of events were summarized for each treatment |
| | arm, all VK2809 treatment arms combined, placebo, and by |
| | SOC and PT. |
| | An overview table summarized the number and percentage of |
| | participants with at least 1 AE and included: |
| | any AE, TEAEs, SAEs |
| | TEAEs and SAEs by severity (Grades 1 to 5) |
| | TEAEs and SAEs by relationship to study drug |
| | (definitely related, probably related, possibly related, |
| | unlikely related, and unrelated) |
| | TEAEs and SAEs by relationship to study procedure |
| | (definitely related, probably related, possibly related, |
| | unlikely related, and unrelated) |
| | TEAEs and SAEs leading to interruption of study drug |
| | TEAEs and SAEs leading to treatment discontinuation |
| | of study drug |
| | TEAEs and SAEs leading to discontinuation of study |
| | SAEs leading to death |
| | treatment-related SAEs leading to death |
| | The number and percentage of participants reporting each AE |
| | were summarized by SOC and PT. PTs were sorted by |
| | descending overall total. The following summaries were |
| | produced: |
| | All AEs, TEAEs, and SAEs by SOC and PT |
| | TEAEs by PT |
| | TEAEs and SAEs related to treatment, by SOC and |
| | PT |
| | TEAEs by relationship to treatment, by SOC and PT |
| | TEAEs by maximum severity, by SOC and PT |
| | TEAEs with severity ≥ Grade 3, by SOC and PT |
| | TEAEs with severity ≥ Grade 4, by SOC and PT |
| | TEAEs related to treatment by maximum severity, by |
| | SOC and PT |
| | TEAEs leading to discontinuation from treatment, by |
| | SOC and PT |
| | TEAEs related to treatment leading to discontinuation |
| | from treatment, by SOC and PT |
| | TEAEs in >5% of participants in any treatment arm by |
| | SOC and PT |
| | SAEs leading to death, by SOC and PT |
| | SAEs in participants who failed screening, by SOC |
| | and PT |
| Laboratory | Laboratory data were summarized by treatment arm and visit |
| Evaluations | with descriptive statistics. Change and percent change from |
| (SAP Section 7.7.3) | baseline were summarized. Pregnancy tests and alcohol and |
| | drug tests were presented as participant listings. |
| | For hematology and serum chemistry, shift tables from |
| | baseline to each scheduled postbaseline visit were provided |
| | for each treatment arm. Corresponding shift tables (normal |
| | versus high/normal versus abnormal) were produced for |
| | urinalysis. |
| | Elevations in liver panel tests were summarized with the |
| | frequency and percentage of instances at each visit by |
| | treatment arm. Summaries of maximum treatment-emergent |
| | bilirubin elevations by treatment arm were provided for |
| | participants who have transaminase elevations (i.e., ALT >3 × |
| | ULN, AST >3 × ULN, alkaline phosphatase >1.5 × ULN). |
| | eDISH plots were generated for peak total bilirubin versus |
| | peak ALT and peak AST relative to the multiple of ULN for |
| | each analyte. The maximum shift of each analyte as a factor |
| | relative to the reference range was presented and plotted on |
| | the log10 scale. |
| | Participants who met potential Hy's Law criteria were |
| | presented. |
| Vital Signs | Individual vital sign measurements were listed and |
| (SAP Section 7.7.4) | summarized by treatment arm and visit. The investigator's |
| | overall assessment (normal; abnormal, clinically significant; |
| | or abnormal, not clinically significant) of the vital sign |
| | measurements at each visit was included. All data including |
| | unscheduled measurements were included. |
| | Vital signs data and changes from baseline in vital signs were |
| | summarized by visit with standard descriptive statistics. For |
| | postbaseline, only data from scheduled visits were included in |
| | the summary tables. |
| Electrocardiograms | Quantitative ECG measurements taken during the study |
| (SAP Section 7.7.5.1) | included heart rate, RR interval, PR interval, QRS interval, |
| | QT interval, Bazett's corrected QT (QTcB) interval, and |
| | Fridericia's corrected interval (QTcF). |
| | The average of the ECG measurements at each visit and |
| | changes from baseline in ECG were summarized by treatment |
| | arm and visit with standard descriptive statistics. All triplicate |
| | values and their average were listed for each parameter. |
| | An overall investigator assessment of ECG was provided |
| | (normal, abnormal, not clinically significant, and abnormal, |
| | clinically significant). |
| | The investigator assessments were listed and the number and |
| | percentage of participants within each assessment category |
| | (worst assessment of each triplicate) were tabulated by |
| | treatment arm and visit. Shifts from baseline at each |
| | postbaseline visit were presented. |
| | The number and percentage of participants with absolute |
| | QTcB and QTcF interval values in the prespecified categories |
| | (>450, >480, and >500 msec), and QTcB and QTcF interval |
| | change (>30 and >60 msec) were summarized by treatment |
| | arm and visit. |
| 24-Hour Holter | Holter monitoring was measured for 24 hours at screening. |
| Monitoring | An overall investigator assessment of the Holter monitoring |
| (SAP Section 7.7.5.2) | data was listed with the number and percentage of |
| | participants within each assessment category tabulated by |
| | treatment arm and visit. Shifts from baseline at each |
| | postbaseline visit were presented. |
| Physical Examination | Physical examination results (normal/abnormal) and details of |
| (SAP Section 7.7.6) | abnormalities were listed for each participant. |

All analyses of the primary and secondary efficacy endpoints were performed with the FAS. The PPS was used for sensitivity analyses where noted. Table 9 below shows the efficacy analysis methods:

TABLE 9

| Endpoint | Methods |
|---|---|
| Primary Efficacy | An ANCOVA model with the relative change in liver fat |
| Analysis | content from baseline to Week 12 as the response variable |
| (SAP Section 7.6.1) | was used to analyze relative change in liver fat content from |
| | baseline to Week 12 in participants treated with VK2809 |
| | compared to the change in participants treated with placebo. |
| | Estimate and contrast statements within the SAS MIXED or |
| | generalized linear model procedure were used to estimate the |
| | mean difference (point estimate), 95% confidence interval, |
| | and associated p-value for the mean differences at Week 12 |
| | between each active treatment arm and placebo. The overall |
| | model p-value based on the type 3 sums of squares was |
| | included in the summary table. |
| | A tipping point analysis tested the assumption of data missing |
| | at random for the primary endpoint in if the primary analysis |
| | null hypothesis was rejected at the 2-sided 5% level. |
| | Participants who were randomized to the active arms with no |
| | MRI-PDFF data at Week 12 were given a penalty. The early |
| | termination MRI-PDFF which was assessed after 8 weeks of |
| | treatment and no later than 4 weeks after the last dose date |
| | was considered as a non-missing Week 12 MRI-PDFF for the |
| | early termination unrelated to an AE related to study drug. |
| | However, participants who terminated treatment prematurely |
| | after Week (12 + 4) also received a penalty. |
| | The penalty increased by increments of 1% until the |
| | conclusion regarding the hypothesis test changed, and the |
| | tipping point was defined as the lowest level of increment at |
| | which this occurs. |
| Secondary Efficacy | ANCOVA models were used to evaluate the absolute and |
| Analysis | relative change from baseline to Week 52 in overall hepatic |
| (SAP Section 7.6.2) | fat fraction (assessed by MRI-PDFF). |
| | The proportion of participants with a relative percent decrease |
| | in liver fat content ≥30% (assessed by MRI-PDFF) from |
| | baseline to Week 12 and Week 52 in participants treated with |
| | VK2809 compared to the proportion of participants with this |
| | decrease who were treated with placebo were analyzed with |
| | logistic regression models. |
| | Secondary endpoints for liver biopsies were analyzed by |
| | CMH by treatment arm |
| Sensitivity Analysis | Analyses of the primary and secondary endpoints were |
| (SAP Section 7.6.3) | repeated for the PPS and the FAS without imputation for |
| | missing data. |
| Subgroup Analysis | The primary and secondary efficacy analyses were repeated |
| (SAP Section 7.6.4) | separately for each level of randomization stratification |
| | factors as follows: |
| | Participants with diabetes status = Y, N |
| | Participants with fibrosis stage = F1, F2, or F3 |
| Exploratory Analysis | Changes and percent changes in the assessment of fibrosis |
| (SAP Section 7.6.5) | and steatosis with dual-photon microscopy were analyzed |
| | with the ANCOVA model. Change from baseline in Ishak |
| | fibrosis score was summarized by treatment arm. |
| | Results of the blinded comparison review of liver biopsies |
| | (Week 52 vs baseline biopsy) at Week 52 included presence |
| | or absence of NASH and values of better, same, or worse for |
| | overall NASH; steatosis; hepatocyte ballooning; |
| | inflammation; and fibrosis. The results of the comparison |
| | review were summarized by parameter and treatment arm. |
| | Additional exploratory endpoints were analyzed with the |
| | CMH method by treatment arm. |

An interim analysis of safety and efficacy data was conducted to determine whether any treatment arms could be discontinued. No formal stopping rules were built into the protocol for early efficacy as the study would continue to Week 52 for additional efficacy evaluations. The goal of the interim analysis was to choose which active arms (after the curtailment of the 1.0 mg QD arm per Amendment 8) should continue to randomize participants for treatment. If an arm was discontinued, the participants would remain in the trial on the same dose, but no further participants would be randomized to that arm. This analysis focused on the primary efficacy endpoint. Each active arm was compared to placebo according to the analysis.

Interim analysis results were reviewed by the unblinded external team. The unblinded results were distributed to a designated unblinded recipient. The study management team, investigators, and participants remained blinded until the study was complete and the final data in the clinical database were locked.

Up to 285 participants were planned to be enrolled and randomized to 1.0 mg QD, 2.5 mg QD, 5.0 mg QOD, 10.0 mg QOD, or placebo in a 1:2:2:2:2 ratio. Randomization was stratified by gender (male, female), fibrosis stage (F1, F2/F3) and diabetes status (yes, no). The sample size estimation was based on the analysis of the mean difference in the percent change in liver fat content from baseline to week 12, evaluated between each of the 4 active arms and placebo. While the overall type-1 error for the primary efficacy analysis was assumed to be 5%, 2-sided, each separate pairwise test of the active drug versus placebo was assumed at the 0.025 level without adjustment for multiplicity. With 75 participants per 2.5 mg QD, 5.0 mg QOD, 10.0 mg QOD, and placebo arms, each test would have 90% power to detect the minimum mean difference of 15%. While the expected mean % change from baseline to week 12 was estimated to be −10% and −40% in the placebo and active arms, respectively (based on the available results from Madrigal and GS-0976 studies) (Harrison S A, Guy C D, Bashir M, Pablo J, Frias, Alkhouri N, et al. In a placebo controlled 36 weeks phase 2 trial, treatment with MGL-3196 compared to placebo results in significant reductions in hepatic fat (MRI-PDFF), liver enzymes, fibrosis biomarkers, atherogenic lipids, and improvement in NASH on serial liver biopsy. Plenary presentation NASDAQ, 2018; Loomba R, Kayali Z, Noureddin M, et al. GS-0976 Reduces hepatic steatosis and fibrosis markers in patients with nonalcoholic fatty liver disease. Gastroenterology. 2018; 155(5): 1463-1473.e6. doi: 10.1053/j.gastro.2018.07.027), the planned sample size was inflated to allow sufficient power to detect differences in the select key secondary analyses.

With respect to the secondary endpoint of proportion of participants whose % change from baseline to week 12 in liver fat content is at least 30%, the above planned sample size, and assuming a placebo rate of 18.4%, the study would have over 90% power to detect a minimum difference of 31%.

With respect to the secondary endpoint of proportion of participants with resolution of NASH/MASH and no worsening of liver fibrosis, the study would have 80% power to detect a 20% difference in proportions, assuming the placebo response rate was 9% as previously reported.

Upon curtailment of the 1.0 mg QD arm, participants were not randomized to that arm. Approximately 20 participants were anticipated to be randomized to this arm. Thereafter, participants were randomized to the remaining arms in a 1:1:1:1 ratio. If other active treatment arms were discontinued after the interim analysis, newly randomized participants were evenly randomized to the remaining arms (e.g., 1:1:1, if 1 arm was discontinued after the interim analysis).

Table 10 shows the protocol amendments applicable to the Phase 2B Clinical Study:

TABLE 10

| | |
|---|---|
| Amendment 8 | Updated: |
| 13 Sep. 2021 | Inclusion/exclusion criteria; |
| | Concomitant medications that prolong QT interval; |
| | Blister pack use; |
| | Ishak fibrosis score; |
| | Exploratory endpoints pertaining to NASH and liver fat |
| | changes; |
| | Laboratory assessments; |
| | Time requirements for vital signs and ECGs; |
| | 1.0 mg QD arm deemed complete; |
| | Holter monitoring; |
| | Remote study visits. |
| Amendment 9 | Updated: |
| 11 Nov. 2021 | Remove 1.0 mg QD arm from study with focus on |
| | placebo, 10.0 mg QD, 5.0 mg QOD, or 2.5 mg QD arms |
| | with corresponding number of enrolled participants; |
| | Planned interim analysis objectives; |
| | Sample size calculation for % of participants with |
| | resolution of NASH without worsening liver fibrosis; |
| | Statistical test specification for CMH method and |
| | determination of sample size; |
| | Role of the DMC in interim analysis; |
| | Laboratory assessments; |
| | Remote visits. |
| Amendment 10 | Updated: |
| 9 Mar. 2022 | Randomization to the 5.0 mg QOD arm after closure |
| | of 1.0 mg QOD arm; |
| | % of participants with an absolute reduction ≥5% in |
| | liver fat content; |
| | Conditions when repeat liver testing is needed; |
| | Definition of study epochs; |
| | Role of DMC in interim analysis; |
| | Statistical analysis of covariance model; |
| | Number of participants enrolled into 5 treatment arms, |
| | in the 5.0 mg arm, and estimated to complete study. |

The SAP (final version 1.0, 22 Dec. 2021) was written from Protocol Amendment 9. Changes in the planned analysis relevant to Amendment 10 included: the analysis of covariance (ANCOVA) model included study epoch (before/after the interim analysis) as a covariate for the final study analysis.

After Amendment 8, Holter monitoring after the screening visit was removed. Thus, analysis of planned time points described in the ECG Analysis SAP (final version 1.0, 12 Apr. 2022) for 24-hour continuous Holter monitoring beyond the screening visit (i.e., Day 1, Week 4, and Week 28) were not included in the final analysis.

In addition to the planned analyses, more conservative sensitivity analyses were added to the final data after the final study data blind break. These analyses categorized participants with missing biopsy data as non-responders. Sensitivity analyses applied to four biopsy and exploratory endpoints.

Secondary endpoints included the following analyses:
Proportion of participants with resolution of steatohepatitis (NASH/MASH) on overall histopathological reading and no worsening of liver fibrosis on NASH CRN fibrosis score;
Proportion of participants with improvement in liver fibrosis greater than or equal to one stage (NASH CRN fibrosis score) and no worsening of steatohepatitis;
Proportion of participants with >2-point improvement in NAS (with at least 1-point improvement in ballooning or inflammation) with no worsening of fibrosis stage;
Proportion of participants with resolution of steatohepatitis and improvement in fibrosis. Improvement is represented by a decline in fibrosis score.

Exploratory endpoints included the following analyses:
Proportion of participants with at least 2 points reduction in NAS;
Proportion of participants with at least 2 points reduction in NAS and resolution of steatohepatitis;
Proportion of participants with at least 2 points reduction in NAS and 1 stage improvement in fibrosis score;
Proportion of participants with at least 1 stage improvement in fibrosis score.

All efficacy analyses were performed on the FAS. The data were also analyzed with the PPS which is considered the observed dose population.

The primary efficacy endpoint is relative change in liver fat content (assessed by MRI PDFF) from baseline to Week 12. Data were imputed for participants with a missing Week 12 assessment to include data from another postbaseline assessment. The primary efficacy endpoint, relative change in liver fat content (assessed by MRI PDFF) from baseline to Week 12. Week 12 and Week 52 data are presented in Table 11.

Notably, all treatment arms had a reduction in liver fat at Week 12 but were more pronounced in the VK2809 arms. Comparing VK2809 versus placebo at Week 12, participants in the VK2809 arms demonstrated statistically significant reductions (p<0.0001) in the absolute and relative percent change from baseline in liver fat. The mean relative reduction ranged from 19.3% to 53.0% in the VK2809 arms. Reductions increased with increasing dose. The corresponding least squares mean (LSM) of the relative change from baseline in all VK2809 arms was 42.3 versus 27.7 for the placebo arm.

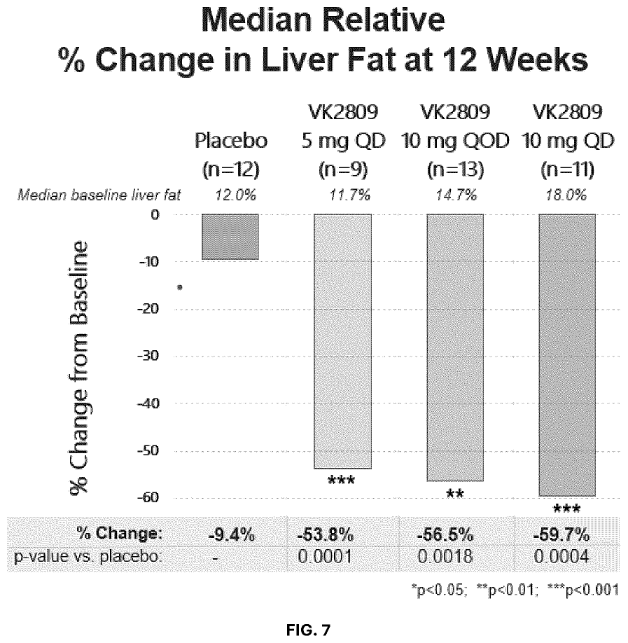

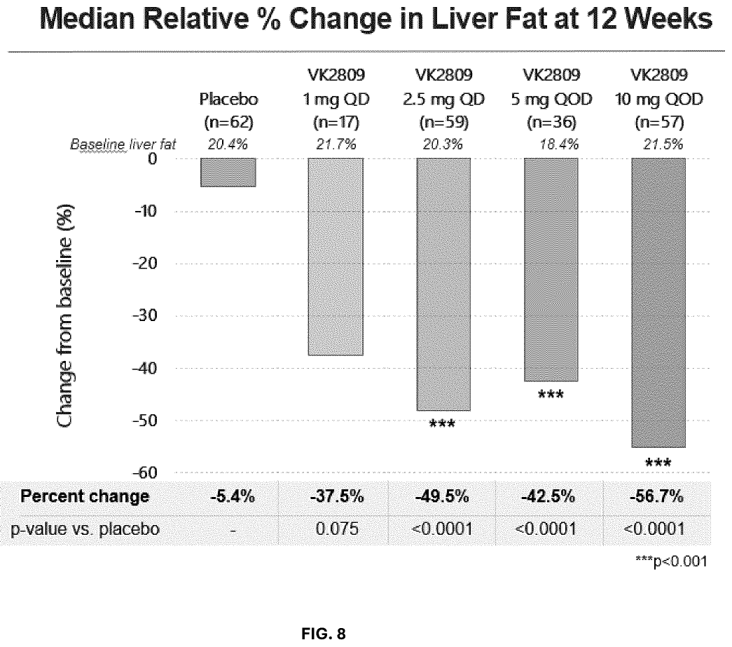

As shown in FIGS. 7 and 8, statistically significant reductions in liver fat were observed from baseline to Week 12 in the higher 3 dose VK2809 arms. See effects of 5.0 mg QD, 10.0 mg QOD, and 10 mg QD shown in FIG. 7, and 2.5 mg QD, 5.0 mg QOD, and 10.0 mg QOD shown in FIG. 8. Overall, there was a significant liver fat reduction observed at 12 weeks. The p-value (p=0.0504) in the 1.0 mg VK2809 arm (FIG. 8) was also reduced and trended near statistical significance. The median relative percentage change in liver fat at 12 weeks for 1 mg QD dose showed a 37.5% decrease, the 2.5 mg QD dose showed a 49.5% decrease, the 5 mg QD dose showed a 53.8% decrease, the 5 mg QOD dose showed a 42.5% decrease, the 10 mg QOD showed a 56.6% decrease, and the 10 mg QD showed a 59.7% decrease. The results show an up to 57% median reduction of liver fat and an overall liver fat effect similar to prior 12-week NAFLD studies.

A tipping point analysis tested the assumption of data missing at random for the primary endpoint if the null hypothesis was rejected at the 2-sided 5% level (2.5% for each pairwise test). Comparing the VK2809 10.0 mg QOD arm versus the placebo arm, absolute liver fat was reduced (p≤0.0089) from baseline to Week 12 in LSM & from 0% to 80%. The tipping point was 81% and higher in which no further statistically significant reductions were observed.

Secondary efficacy endpoints assessed mean hepatic fat fraction (MHFF) by MRI-PDFF, and liver fibrosis, steatohepatitis, and NASH/MASH by liver biopsy from baseline and at weeks 12 and 52. Alternative methods for assessing liver fibrosis, steatohepatitis, and/or NASH include magnetic resonance elastography (MRE) and transient elastography (FibroScan) in addition to liver biopsy, among other methods to determine fibrosis, steatophepatitis, and/or NASH/MASH. Secondary efficacy endpoints assessed MHFF by MRI-PDFF at weeks 12 and 52. Data were imputed for participants with a missing Week 52 assessment to include data from another postbaseline assessment. An overview of liver fat content at weeks 12 and 52 is shown in Table 12.

Greater reductions in liver fat were noted in participants in the VK2809 arms versus the placebo arm at Week 52. Notably all treatment arms had a reduction in liver fat at Week 52 but were more pronounced in the VK2809 arms. Further, the liver fat reduction was maintained, based on the 12 week results, at 52 weeks.

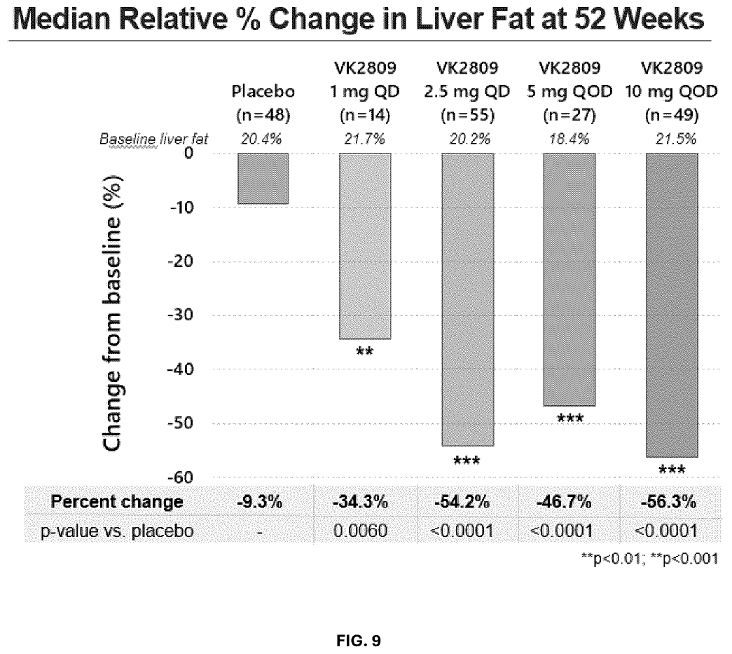

As shown in FIG. 9, there was significant liver fat reduction maintained at 52 weeks compared to 12 weeks. In more detail, significant median relative percentage change in liver fat was maintained for 1 mg QD dose, 2.5 mg QD dose, 5 mg QOD dose, and 10 mg QOD dose. The median relative percent change in liver fat at 52 weeks for the 1 mg QD dose was 34.3% reduction, the 2.5 mg QD dose was 54.2% reduction, the 5 mg QOD was 46.7% reduction, and the 10 mg QOD was 56.3% reduction. Overall, there was up to 56% median reduction and maintained in liver fat. The results show a sustained liver fat reduction indicating a robust durability in treatment effects.

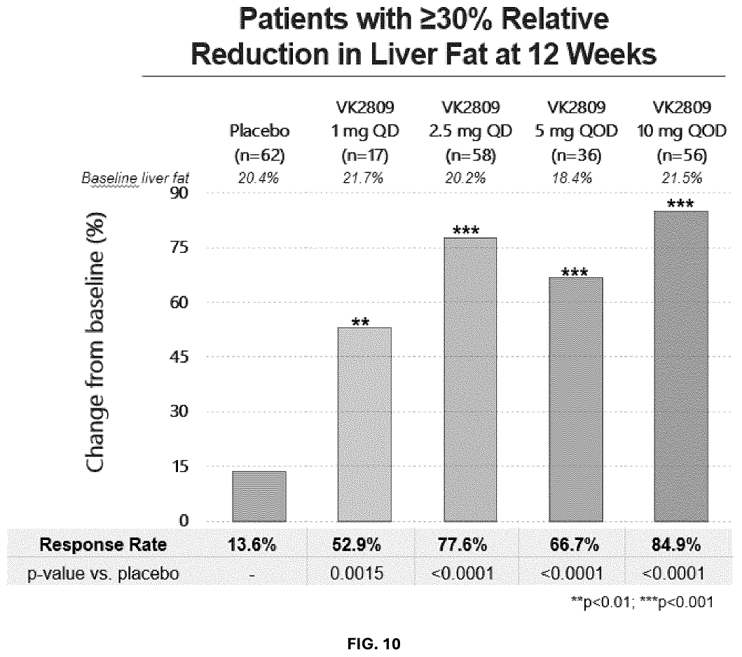

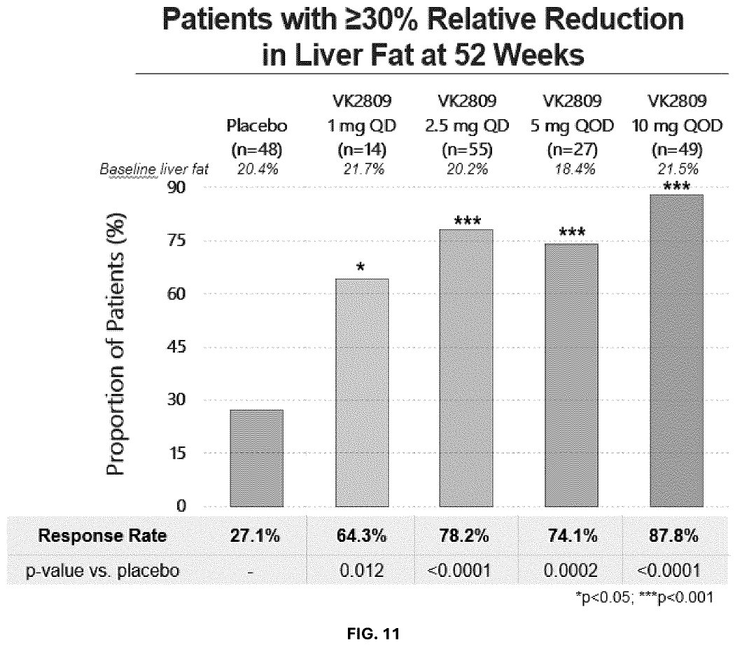

Further, FIG. 10 shows the patients with >30% relative reduction in liver fat at 12 weeks and FIG. 11 shows the patients with >30% relative reduction in liver fat at 52 weeks.

As shown in FIG. 10, there was a significant change from baseline percentage in liver fat at 12 weeks for the 1 mg QD dose, the 2.5 mg QD dose, the 5 mg QOD dose, and 10 mg QOD dose. The relative reduction in liver fat for 1 mg QD dose was 52.9%, the 2.5 mg QD dose was 77.6%, the 5 mg QOD dose is 66.7%, and the 10 mg QOD dose was 84.9%. Overall, up to 85% of VK2809 patients experienced a response, as defined by >30% relative reduction in liver fat at week 12. The combined VK2809 cohorts demonstrated a 75% response rate. The reduction in liver fat was correlated with improved odds of long-term histology benefits.

As shown in FIG. 11, there was a significant change from baseline percentage in liver fat at 52 weeks for the 1 mg QD dose, the 2.5 mg QD dose, the 5 mg QOD dose, and 10 mg QOD dose. Further, the high response rate of liver fat reduction was maintained at the 52 weeks from the 12 week results. As shown in FIG. 11, the relative reduction in liver fat for 1 mg QD dose was 64.3%, the 2.5 mg QD dose was 78.2%, the 5 mg QOD dose is 74.1%, and the 10 mg QOD dose was 87.8%. Overall, up to 88% of VK2809 patients experienced a response, as defined by >30% relative reduction in liver fat at week 52. The combined VK2809 cohorts demonstrated a 79% response rate. The reduction in liver fat was correlated with improved odds of long-term histology benefits.

The absolute and relative LSM change in liver fat at week 12 and week 52 and the response rate for ≥30% fat reduction to Week 52 in all VK2809 arms versus the placebo arm are presented below:
Absolute LSM change from baseline to Week 12 in all VK2809 arms (LSM=8.38; LSM difference=6.95; p<0.0001);
Relative LSM change from baseline to Week 12 in all VK2809 arms (LSM=38.17 difference 34.18; p<0.0001);
Response rate of ≥30% fat reduction to Week 12 for all VK2809 arms versus placebo (response rate=75.3% vs.13.6%; p<0.0001);
Relative mean reduction in overall liver fat content at Week 52 ranged from 30.1% to 53.0% in the VK2809 arms. Reductions increased with increasing dose;
Absolute mean reduction in overall liver fat content at Week 52 ranged from 6.57% to 12.0% in the VK2809 arms. Reductions increased with increasing dose;
Absolute LSM change from baseline to Week 52 for VK2809 arms (LSM=42.45; LSM difference=34.2; p<0.0001);
Relative LSM change from baseline to Week 52 for VK2809 arms (LSM=9.24; LSM difference=4.85; p<0.0001);
Response rate of >30% fat reduction to Week 52 for VK2809 arms (response rate=79.3% versus 27.1%; p<0.0001).

Similar relative and absolute changes from baseline in weeks 12 and 52 were observed for the FAS without imputation and the PPS. Decreases in fat reduction ≥30% from baseline were present in the PPS at Week 12 and Week 52. Moreover, ≥50% decreases in fat reduction were presented in the PPS at Week 12 and Week 52.

TABLE 11

| | | | | | |
|---|---|---|---|---|---|
| Placebo QD | 1.0 mg QD | 2.5 mg QD | 5.0 mg QOD | 10.0 mg QOD | All VK2809 |
| (N = 62) | (N = 17) | (N = 59) | (N = 36) | (N = 57) | (N = 169) |

Overall Mean Hepatic Fat Fraction (%) [1]
Baseline

| n | 62 | | 17 | | 59 | | 36 | | 57 | | 169 | |
|---|---|---|---|---|---|---|---|---|---|---|---|---|
| Mean (SD) | 20.39 | (7.444) | 21.73 | (8.447) | 20.33 | (7.834) | 18.43 | (6.603) | 21.46 | (8.420) | 20.45 | (7.879) |

| | | | | | | |
|---|---|---|---|---|---|---|
| Median | 19.70 | 20.10 | 19.20 | 15.80 | 21.60 | 19.60 |
| Min, Max | 8.2, 39.7 | 12.2, 39.2 | 8.1, 43.9 | 8.4, 31.0 | 8.1, 40.7 | 8.1, 43.9 |

Week 12

| n | 62 | | 17 | | 59 | | 36 | | 57 | | 169 | |
|---|---|---|---|---|---|---|---|---|---|---|---|---|
| Mean (SD) | 19.07 | (7.077) | 17.09 | (8.526) | 10.44 | (5.576) | 11.48 | (5.347) | 9.90 | (6.171) | 11.15 | (6.373) |

| | | | | | | |
|---|---|---|---|---|---|---|
| Median | 18.65 | 16.30 | 9.50 | 10.80 | 8.40 | 9.40 |
| Min, Max | 7.1, 41.7 | 3.4, 38.3 | 2.5, 31.3 | 3.6, 22.4 | 1.6, 29.7 | 1.6, 38.3 |

Change from Baseline to Week 12

| n | 62 | | 17 | | 59 | | 36 | | 57 | | 169 | |
|---|---|---|---|---|---|---|---|---|---|---|---|---|
| Mean (SD) | −1.32 | (4.999) | −4.64 | (7.262) | −9.89 | (7.355) | −6.95 | (5.334) | −11.56 | (6.895) | −9.30 | (7.121) |
| Median | −0.95 | | −5.90 | | −8.60 | | −6.80 | | −10.40 | | −8.20 | |
| Min, Max | −13.8, 17.2 | | −19.0, 11.5 | | −30.7, 9.1 | | −21.6, 4.8 | | −27.3, 3.9 | | −30.7, 11.5 | |
| p-value for Change | 0.0414 | | 0.0180 | | <0.0001 | | <0.0001 | | <0.0001 | | <0.0001 | |
| from Baseline [3] | | | | | | | | | | | | |
| LSM (SEM) [4] | −1.43 | (0.66) | −4.23 | (1.31) | −10.05 | (0.67) | −8.13 | (0.97) | −11.09 | (0.69) | −8.38 | (0.53) |

| | | | | | | |
|---|---|---|---|---|---|---|
| 95% CI [4] | −2.733, −0.118 | −6.811, −1.643 | −11.378, −8.727 | −10.050, −6.214 | −12.440, −9.739 | −9.422, −7.328 |

Treatment Comparison (VK2809 - Placebo)

| | | | | | | | | | | | |
|---|---|---|---|---|---|---|---|---|---|---|---|
| LSM Difference | NA | −2.80 | (1.42) | −8.63 | (0.91) | −6.71 | (1.13) | −9.66 | (0.92) | −6.95 | (0.79) |
| (SEM) [4] | | | | | | | | | | | |

| | | | | | | |
|---|---|---|---|---|---|---|
| 95% CI [4] | NA | −5.607, 0.005 | −10.415, −6.839 | −8.934, −4.479 | −11.479, −7.848 | −8.512, −5.386 |
| p-value [4] | NA | 0.0504 | <0.0001 | <0.0001 | <0.0001 | <0.0001 |

Percent Change from Baseline to Week 12 [2]

| n | 62 | | 17 | | 59 | | 36 | | 57 | | 169 | |
|---|---|---|---|---|---|---|---|---|---|---|---|---|
| Mean (SD) | −3.96 | (27.499) | −19.30 | (38.527) | −45.89 | (25.449) | −36.51 | (25.542) | −52.96 | (21.564) | −43.60 | (27.531) |
| Median | −5.42 | | −37.50 | | −49.45 | | −42.53 | | −56.71 | | −45.77 | |
| Min, Max | −48.6, 145.8 | | −72.1, 94.3 | | −88.5, 41.0 | | −73.2, 31.6 | | −88.7, 18.4 | | −88.7, 94.3 | |
| p-value % Change | 0.2616 | | 0.0555 | | <0.0001 | | <0.0001 | | <0.0001 | | <0.0001 | |
| from BL [3] | | | | | | | | | | | | |
| LSM (SEM) [4] | −3.99 | (3.46) | −17.15 | (6.75) | −45.95 | (3.51) | −37.27 | (4.88) | −52.32 | (3.56) | −38.17 | (2.70) |

| | | | | | | |
|---|---|---|---|---|---|---|
| 95% CI [4] | −10.808, 2.818 | −30.450, −3.856 | −52.857, −39.038 | −46.881, −27.665 | −59.332, −45.318 | −43.500, −32.850 |

Treatment Comparison (VK2809 - Placebo)

| | | | | | | | | | | | |
|---|---|---|---|---|---|---|---|---|---|---|---|
| LSM Difference | NA | −13.16 | (7.35) | −41.95 | (4.73) | −33.28 | (5.75) | −48.33 | (4.79) | −34.18 | (4.10) |
| (SEM) [4] | | | | | | | | | | | |

| | | | | | | |
|---|---|---|---|---|---|---|
| 95% CI [4] | NA | −27.646, 1.329 | −51.279, −32.626 | −44.606, −21.950 | −57.767, −38.894 | −42.269, −26.091 |
| p-value | NA | 0.0109 | <0.0001 | <0.0001 | <0.0001 | <0.0001 |

Week 52

| n | 62 | | 17 | | 59 | | 36 | | 57 | | 169 | |
|---|---|---|---|---|---|---|---|---|---|---|---|---|
| Mean (SD) | 17.70 | (7.363) | 15.16 | (7.764) | 9.73 | (5.214) | 10.91 | (4.593) | 9.46 | (5.712) | 10.44 | (5.762) |

| | | | | | | |
|---|---|---|---|---|---|---|
| Median | 17.60 | 13.70 | 9.50 | 11.00 | 9.00 | 9.50 |
| Min, Max | 3.6, 42.5 | 4.1, 31.9 | 1.5, 29.0 | 3.6, 21.9 | 2.1, 29.5 | 1.5, 31.9 |

Change from Baseline to Week 52

| n | 62 | | 17 | | 59 | | 36 | | 57 | | 169 | |
|---|---|---|---|---|---|---|---|---|---|---|---|---|
| Mean (SD) | −2.69 | (6.298) | −6.57 | (6.270) | −10.60 | (8.804) | −7.52 | (5.675) | −12.00 | (8.135) | −10.01 | (7.954) |
| Median | −1.75 | | −5.10 | | −10.20 | | −8.05 | | −11.20 | | −9.20 | |
| Min, Max | −22.1, 7.9 | | −21.3, 3.3 | | −39.4, 5.7 | | −21.7, 6.6 | | −33.5, 7.1 | | −39.4, 7.1 | |
| p-value for Change | 0.0014 | | 0.0005 | | <0.0001 | | <0.0001 | | <0.0001 | | <0.0001 | |
| from BL [3] | | | | | | | | | | | | |
| LSM (SEM) [4] | −2.73 | (0.70) | −6.17 | (1.39) | −10.74 | (0.71) | −8.79 | (1.03) | −11.26 | (0.73) | −9.24 | (0.56) |

| | | | | | | |
|---|---|---|---|---|---|---|
| 95% CI [4] | −4.120, −1.348 | −8.906, −3.427 | −12.147, −9.335 | −10.824, −6.756 | −12.695, −9.831 | −10.350, −8.130 |

Treatment Comparison (VK2809 - Placebo)

| | | | | | | | | | | |
|---|---|---|---|---|---|---|---|---|---|---|
| LSM Difference | −3.43 | (1.51) | −8.01 | (0.96) | −6.06 | (1.20) | −8.53 | (0.98) | −6.51 | (0.84) |
| (SEM) [4] | | | | | | | | | | |

| | | | | | |
|---|---|---|---|---|---|
| 95% CI [4] | −6.408, −0.458 | −9.904, −6.111 | −8.418, −3.694 | −10.454, −6.605 | −8.164, −4.849 |
| p-value [4] | 0.0239 | <0.0001 | <0.0001 | <0.0001 | <0.0001 |

Percent Change from Baseline to Week 52 [2]

| n | 62 | | 17 | | 59 | | 36 | | 57 | | 169 | |
|---|---|---|---|---|---|---|---|---|---|---|---|---|
| Mean (SD) | −10.00 | (29.557) | −30.08 | (23.981) | −46.44 | (29.815) | −37.67 | (26.884) | −53.01 | (25.388) | −45.14 | (27.993) |
| Median | −7.77 | | −30.19 | | −53.26 | | −41.49 | | −55.94 | | −49.21 | |
| Min, Max | −78.9, 54.1 | | −66.4, 14.7 | | −92.6, 26.0 | | −71.0, 67.3 | | −91.0, 31.7 | | −92.6, 67.3 | |
| p-value for Percent | 0.0099 | | <0.0001 | | <0.0001 | | <0.0001 | | <0.0001 | | <0.0001 | |
| Change from BL [3] | | | | | | | | | | | | |
| LSM (SEM) [4] | −10.61 | (3.52) | −29.84 | (6.87) | −46.99 | (3.57) | −40.84 | (4.96) | −52.10 | (3.62) | −42.45 | (2.75) |

| | | | | | | |
|---|---|---|---|---|---|---|
| 95% CI [4] | −17.552, −3.676 | −43.383, −16.302 | −54.031, −39.958 | −50.629, −31.061 | −59.239, −44.969 | −47.869, −37.024 |

Treatment Comparison (VK2809 - Placebo)

| | | | | | | | | | | |
|---|---|---|---|---|---|---|---|---|---|---|
| LSM Difference | −19.23 | (7.49) | −36.38 | (4.82) | −30.23 | (5.85) | −41.49 | (4.88) | −31.83 | (4.18) |
| (SEM) [4] | | | | | | | | | | |

| | | | | | |
|---|---|---|---|---|---|
| 95% CI [4] | −33.982, −4.476 | −45.878, −26.883 | −41.767, −18.695 | −51.099, −31.881 | −40.069, −23.595 |
| p-value [4] | 0.0109 | <0.0001 | <0.0001 | <0.0001 | <0.0001 |

Abbreviations: ANCOVA = analysis of covariance, BL = baseline, CI = confidence interval, LSM = least aquares mean, Max = maximum; MHFF = mean hepatic fat fraction, Max = maximum, Min = minimum, MRI-PDFF = Magnetic Resonance Imaging-Proton Density, NA = not applicable, QD = daily, QOD = every other day
[1] Mean hepatic fat fraction (%) was assessed by MRI-PDFF.
[2] Percent Change from Baseline was computed as (Postbaseline-Baseline)/Baseline*100%.
[3] Paired t-test within treatment group
[4] Results were from an Analysis of Covariance Model (ANCOVA) with randomization stratification factors (including whether the subject was randomized before/after the interim analysis), treatment assignments, baseline MHFF, and interaction of baseline MHFF with treatment as covariates. The interaction of MHFF with treatment was not retained in the model if the p-value for the interaction was greater than the 0.15-level of significance and the model reran before estimates were computed.

TABLE 12

| | Response Rate (%) | | | | | |
|---|---|---|---|---|---|---|
| | Placebo QD | 1.0 mg QD | 2.5 mg QD | 5.0 mg QOD | 10.0 mg QOD | All VK2809 |
| Secondary Endpoints | (N = 62) | (N = 17)⁴ | (N = 59) | (N = 36) | (N = 57) | (N = 169) |

Week 12
Absolute change from Baseline

| | | | | |
|---|---|---|---|---|
| LSM (SEM) ² | −4.54 | (4.332) | 19.49 | (8.752 |

| | | | | | | | | | |
|---|---|---|---|---|---|---|---|---|---|
| 31.51 | (4.219) | 15.27 | (6.190) | 28.28 | (4.354) | −9.66 | (0.92) | −6.95 | (0.79) |

| | | | | | |
|---|---|---|---|---|---|
| p-value ² | 0.0504 | <0.0001 | <0.0001 | <0.0001 | <0.0001 |

Relative % Change from Baseline ¹

| | | | | | | | | | | | | |
|---|---|---|---|---|---|---|---|---|---|---|---|---|
| LSM (SEM) ² | −3.99 | (3.46) | −17.15 | (6.75) | −45.95 | (3.51) | −37.27 | (4.88) | −52.32 | (3.56) | −38.17 | (2.70) |

| | | | | | | | | | | | |
|---|---|---|---|---|---|---|---|---|---|---|---|
| LSM Difference (SEM) ² | NA | −13.16 | (7.35) | −41.95 | (4.73) | −33.28 | (5.75) | −48.33 | (4.79) | −34.18 | (4.10) |

| | | | | | | |
|---|---|---|---|---|---|---|
| p-value ² | NA | 0.0748 | <0.0001 | <0.0001 | <0.0001 | <0.0001 |

Participants with ≥30 fat reduction

| | | | | | | |
|---|---|---|---|---|---|---|
| Response rate (%) | 13.6 | 52.9 | 78.0 | 66.7 | 85.2 | 75.3 |
| 95% CI ³ | 7.03, 24.54 | 30.96, 73.83 | 65.87, 86.65 | 50.33, 79.79 | 73.40, 92.30 | 68.22, 81.24 |
| p-value ² | NA | 0.0014 | <0.0001 | <0.0001 | <0.0001 | <0.0001 |

Week 52
Absolute Change from Baseline

| | | | | | | | | | | | | |
|---|---|---|---|---|---|---|---|---|---|---|---|---|
| LSM (SEM) | −2.73 | (0.70) | −6.17 | (1.39) | −10.74 | (0.71) | −8.79 | (1.03) | −11.26 | (0.73) | −9.24 | (0.56) |

| | | | | | | |
|---|---|---|---|---|---|---|
| LSM Difference (SEM) | NA | −6.408, −0.458 | −9.904, −6.111 | −8.418, −3.694 | −10.454, −6.605 | −8.164, −4.849 |
| p-value | NA | 0.0239 | <0.0001 | <0.0001 | <0.0001 | <0.0001 |

Relative Change from Baseline

| | | | | | | | | | | | | |
|---|---|---|---|---|---|---|---|---|---|---|---|---|
| LSM (SEM) | −10.61 | (3.52) | −29.84 | (6.87) | −46.99 | (3.57) | −40.84 | (4.96) | −52.10 | (3.62) | −42.45 | (2.75) |

| | | | | | | | | | | | |
|---|---|---|---|---|---|---|---|---|---|---|---|
| LSM Difference (SEM) | NA | −19.23 | (7.49) | −36.38 | (4.82) | −30.23 | (5.85) | −41.49 | (4.88) | −31.83 | (4.18) |

| | | | | | | |
|---|---|---|---|---|---|---|
| p-value | NA | 0.0109 | <0.0001 | <0.0001 | <0.0001 | <0.0001 |

Participants with ≥30 fat reduction

| | | | | | | |
|---|---|---|---|---|---|---|
| Response rate (%) | 27.1 | 64.3 | 78.2 | 74.1 | 87.8 | 79.3 |
| 95% CI ³ | 16.57, 41.00 | 38.76, 83.66 | 65.63, 87.05 | 55.32, 86.83 | 75.76, 94.27 | 72.00, 85.11 |
| p-value ² | NA | 0.0116 | <0.0001 | <0.0001 | <0.0001 | <0.0001 |

Abbreviations: MHFF = mean hepatic fat fraction; NA = not applicable; QD = daily; QOD = every other day
¹ Mean hepatic fat fraction (%) is assessed by MRI-PDFF.
² Results were from an Analysis of Covariance Model (ANCOVA) with randomization stratification factors (including whether the subject was randomized before/after the interim analysis), treatment assignments, baseline MHFF, and the interaction of baseline MHFF with treatment as covariates. The interaction of MHFF with treatment is not retained in the model if the p-value for the interaction was greater than the 0.15 level of significance and the model reran before estimates are computed.
³ Wilson confidence interval for response rate.
⁴p-values based on a logistic regression model with randomization stratification factors (including whether the subject was randomized before/after the interim analysis), treatment assignments, baseline MHFF, and the interaction of baseline MHFF with treatment as covariates. The interaction of baseline MHFF with treatment is not retained in the model if the p-value for the interaction term is greater than the 0.15-level of significance, and the model rerun before estimates were computed.

Secondary efficacy endpoints of liver fibrosis, steatohepatitis, and NASH/MASH were assessed by liver biopsy from baseline and at weeks 12 and 52. Resolution of steatohepatitis is defined as absent fatty liver disease or isolated or simple steatosis without steatohepatitis and an NAS score of 0 to 1 for inflammation, 0 for ballooning, and any value for steatosis. A worsening of steatohepatitis is defined as an increase from baseline in ballooning, inflammation, and steatosis when measured via liver biopsy.

Improvement of fibrosis is defined as at least 1-stage improvement from baseline in fibrosis score and no increase from baseline in ballooning, inflammation, and steatosis. A worsening of fibrosis is defined as a higher grade of fibrosis observed versus baseline values via liver biopsy. No worsening of fibrosis is defined as a fibrosis score at Week 52 that is less than or equal to a baseline score.

Results from liver biopsies were provided by a central reader. The criterion for improvement is defined as a lower grade versus baseline by at least 1-stage in fibrosis score and no increase in ballooning, inflammation, or steatosis.

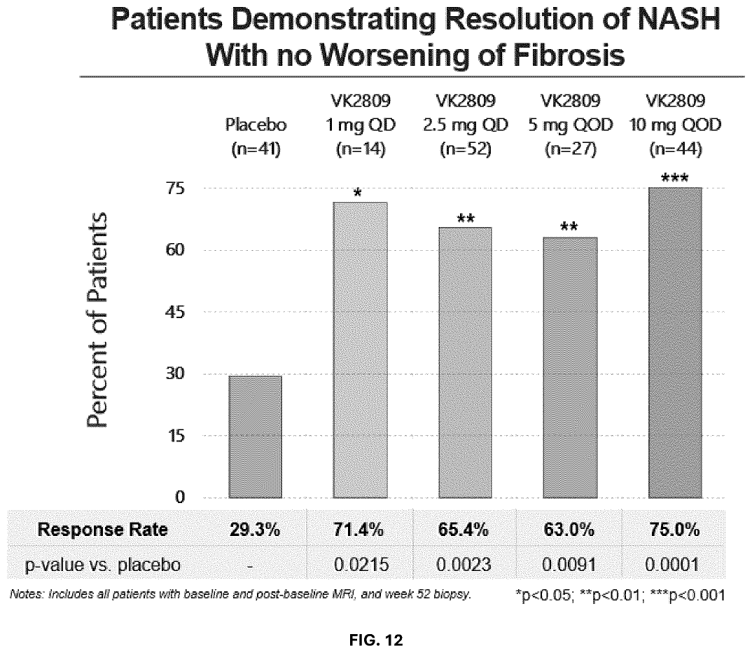

FIG. 12 shows results from the patients demonstrating resolution of NASH/MASH with no worsening of fibrosis. Overall, NASH/MASH resolution was observed in up to 75% of patients. The resolution of NASH/MASH is defined as NAS inflammation score of 0-1 and a ballooning score of 0. A statistically significant percent of patients showed a resolution of NASH/MASH with no worsening of fibrosis for the 1 mg QD dose, the 2.5 mg QD dose, the 5 mg QOD dose, and the 10 mg QOD dose. As shown in FIG. 12, the percent of patients demonstrating resolution of NASH with no worsening of fibrosis for the 1 mg QD dose was 71.4%, the 2.5 mg QD dose was 65.4%, the 5 mg QOD was 63.0%, and the 10 mg QOD dose was 75.0%. The NASH resolution with no worsening of fibrosis is a U.S. regulatory secondary endpoint.

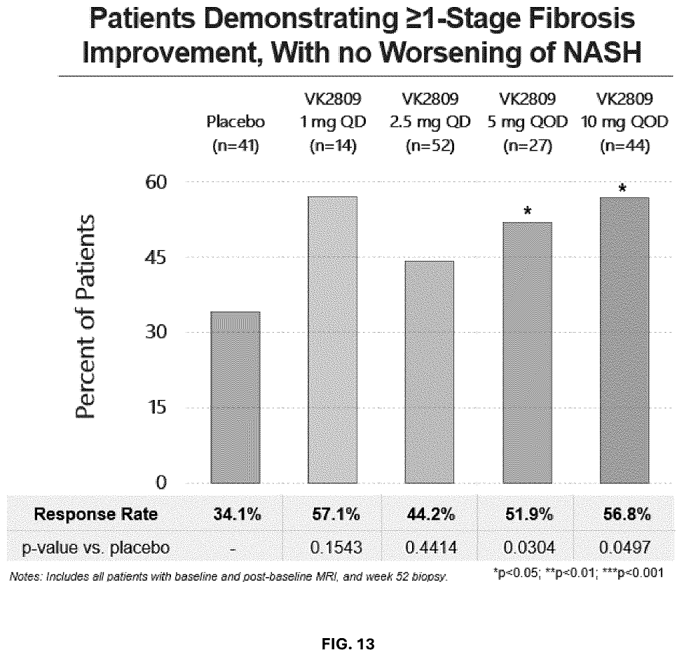

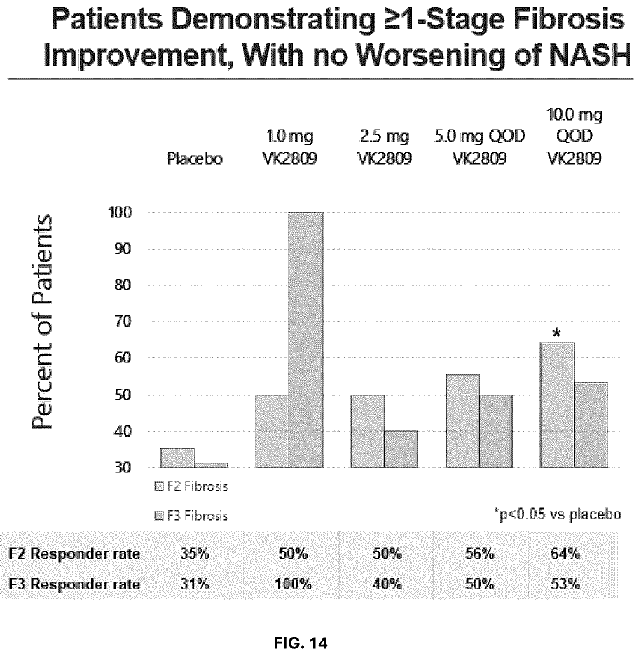

FIGS. 13 and 14 show results from patients demonstrating ≥1-stage fibrosis improvement with no worsening of NASH/MASH. As shown, there was a significant percentage of patients demonstrating >1-stage fibrosis improvement with no worsening of NASH/MASH for the 5 mg QOD dose and the 10 mg QOD dose. FIG. 13 shows the percentage of patients demonstrating >1-stage fibrosis improvement with no worsening of NASH/MASH for the 1 mg QD dose was 57.1%, the 2.5 mg QD dose was 44.2%, the 5 mg QOD dose was 51.9%, and the 10 mg QOD dose is 56.8%. The F2 fibrosis responder rate for the 10.0 mg QOD dose was further significant. FIG. 14 shows the F2 responder rate for the 1.0 mg QD dose was 50%, for the 2.5 mg QD dose was 50%, for the 5.0 mg QOD dose was 56%, and for the 10.0 mg QOD dose was 64%. The F3 responder rate for the 1.0 mg QD dose was 100%, for the 2.5 mg QD dose was 40%, for the 5.0 mg QOD dose was 50%, and for the 10.0 mg QOD dose was 53%. The results show a similar fibrosis improvement rates among the F2 and F3 subsets. The patients demonstrating >1-stage fibrosis improvement with no worsening of NASH/MASH is a U.S. regulatory secondary endpoint.

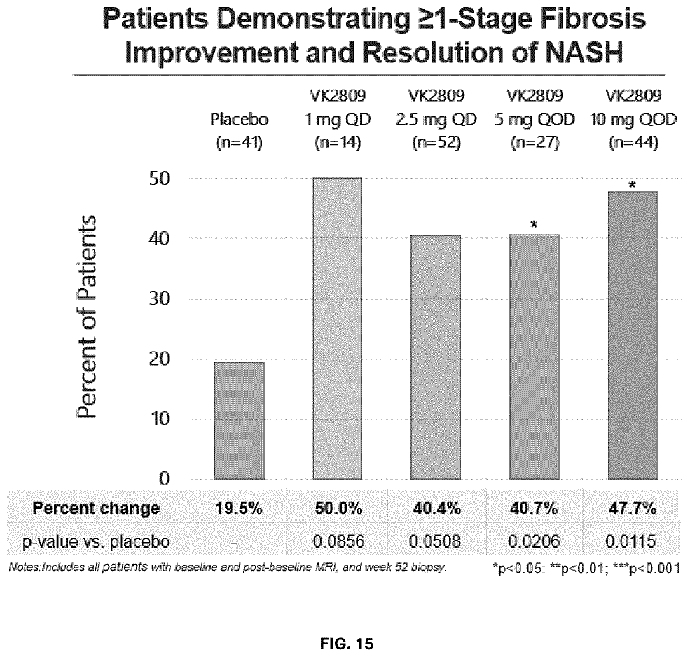

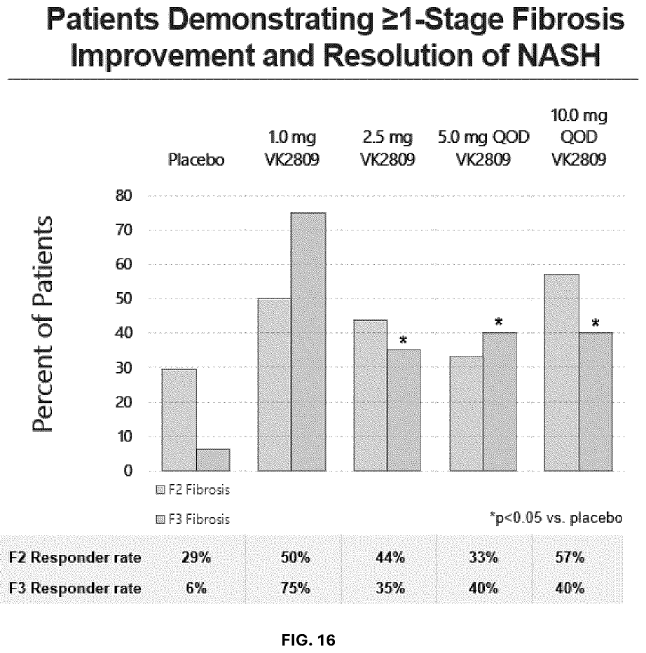

FIGS. 15 and 16 show results from patients demonstrating ≥1-stage fibrosis improvement with resolution of NASH/MASH. As shown, there was a statistically significant percent of patients demonstrating >1-stage fibrosis improvement with resolution of NASH/MASH for the 5 mg QOD dose and the 10 mg QOD dose. The percentage of patients demonstrating >1-stage fibrosis improvement with no worsening of NASH/MASH for the 1 mg QD dose was 50.0%, for the 2.5 mg QD dose was 40.4%, for the 5 mg QOD dose was 40.7%, and for the 10 mg QOD dose was 47.7%. As shown in FIG. 16, the F3 fibrosis responder rate for the 2.5 mg QD dose, the 5.0 mg QOD dose, and the 10.0 mg QOD dose were statistically significant. The F2 responder rate for the 1.0 mg QD dose was 50%, for the 2.5 mg QD dose was 44%, for the 5.0 mg QOD dose was 33%, and for the 10.0 mg QOD dose was 57%. The F3 responder rate for the 1.0 mg QD dose was 75%, for the 2.5 mg QD dose was 35%, for the 5.0 mg QOD dose was 40%, for the 10.0 mg QOD dose was 40%. The results show a similar trend in fibrosis improvement rates among the F2 and F3 subsets.

A summary of change from baseline in fibrosis and NAFLD is shown in Table 13. Improvement in fibrosis and NAFLD parameters (NAS score, steatosis, NAS ballooning, inflammation) to Week 52 was noted for all treatment groups but reductions for each parameter were greater in the VK2809 treatment arms versus the placebo arm.

The mean change in fibrosis from baseline to Week 52 was reduced in all treatment arms: ranges from 0.4 to 0.7 in VK2809 arms versus 0.2 in the placebo arm were noted. Median change from baseline was-1 for VK2809 treatment arms for the 1.0 mg QD, 5.0 mg QOD, and 10.0 mg QOD treatment arms versus 0 for the placebo and the VK2809 2.5 mg arm. Notably, 20.3% of participants in the VK2809 arms improved to stage F0 fibrosis at Week 52 versus 13.0% in the placebo arm. Moreover, fewer participants in the VK2809 arms had stage F3 fibrosis at Week 52 (15.2%) versus baseline (35.5%), with most participants improving. Six participants (4.3%) in the VK2809 arms versus 1 participant (2.2%) in the placebo arm advanced to stage F4 fibrosis.

An overview of the response rate for steatohepatitis and liver fibrosis at week 52 is shown in Table 14. Mean fibrosis and steatohepatitis improved from baseline to Week 52 in all treatment arms but improvement was greater in the VK2809 treatment arms. Improvements for the combined VK2809 treatment arms were noted for all VK2809 treatment arms versus the placebo arm in each parameter as follows:
Resolution of steatohepatitis with no worsening fibrosis (response rate=68.6% vs. 29.3%; p<0.0001);
≥1-stage of fibrosis improvement and no worsening steatohepatitis (response rate=51.1% vs. 34.1%; p=0.0308);
≥2-point NAS improvement with no worsening fibrosis (response rate=75.2% vs. 34.1%; p<0.0001);
Resolution of ≥1-stage steatohepatitis and fibrosis improvement (response rate=43.8% vs. 19.5%; p≤0.0115).

A more conservative sensitivity analysis of response rate for steatohepatitis and liver fibrosis at Week 52 in which participants lacking Week 52 biopsies were considered as non-responders. The ad hoc analyses are shown in Table 15.

Ad hoc analyses were consistent with prior analyses. Mean fibrosis and steatohepatitis improved from baseline to Week 52 in all treatment arms but improvement was greater in the VK2809 treatment arms. Improvements for the combined VK2809 treatment arms were noted for all VK2809 treatment arms versus the placebo arm in each parameter as follows:
Resolution of steatohepatitis with no worsening fibrosis (response rate=56.0% vs. 21.1%; p<0.0001);
≥1-stage of fibrosis improvement and no worsening steatohepatitis (response rate=41.7% vs. 24.6%; p=0.0101);
≥2-point NAS improvement with no worsening fibrosis (response rate=61.3% vs. 24.6%; p<0.0001);
Resolution of ≥1-stage steatohepatitis and fibrosis improvement (response rate=35.7% vs. 14.0%; p=0.0009).

TABLE 13

| | | | | | | |
|---|---|---|---|---|---|---|
| Visit | Placebo QD | 1.0 mg QD | 2.5 mg QD | 5.0 mg QOD | 10.0 mg QOD | All VK2809 |
| Category/Statistics | (N = 62) | (N = 17) | (N = 59) | (N = 36) | (N = 57) | (N = 169) |

Change in fibrosis

| | | | | | | |
|---|---|---|---|---|---|---|
| n | 46 | 14 | 52 | 27 | 45 | 138 |
| Mean (SD) | −0.2 (0.95) | −0.6 (0.84) | −0.4 (1.01) | −0.4 (0.85) | −0.7 (0.75) | −0.5 (0.89) |
| Median | 0.0 | −1.0 | 0.0 | −1.0 | −1.0 | −1.0 |
| Min, Max | −2, 2 | −2, 1 | −3, 2 | −2, 1 | −2, 1 | −3, 2 |

Change in NAS Score

| | | | | | | |
|---|---|---|---|---|---|---|
| n | 47 | 14 | 53 | 27 | 46 | 140 |
| Mean (SD) | −1.1 (1.52) | −2.3 (1.73) | −3.1 (1.43) | −2.6 (1.34) | −3.0 (1.56) | −2.9 (1.50) |
| Median | −1.0 | −3.0 | −3.0 | −3.0 | −3.0 | −3.0 |
| Min, Max | −5, 2 | −4, 1 | −6, 1 | −5, 0 | −6, 2 | −6, 2 |

Change in Steatosis

| | | | | | | |
|---|---|---|---|---|---|---|
| n | 47 | 14 | 54 | 27 | 46 | 141 |
| Mean (SD) | −0.2 (0.97) | −0.6 (0.84) | −1.2 (1.00) | −0.7 (0.91) | −1.2 (0.90) | −1.0 (0.96) |
| Median | 0.0 | 0.0 | −1.0 | −1.0 | −1.0 | −1.0 |
| Min, Max | −2, 2 | −2, 0 | −3, 1 | −3, 1 | −3, 1 | −3, 1 |

Change in NAS Ballooning

| | | | | | | |
|---|---|---|---|---|---|---|
| n | 47 | 14 | 53 | 27 | 46 | 140 |
| Mean (SD) | −0.6 (0.77) | −1.1 (0.77) | −1.3 (0.71) | −1.2 (0.64) | −1.3 (0.76) | −1.3 (0.71) |
| Median | 0.0 | −1.0 | −1.0 | −1.0 | −1.0 | −1.0 |
| Min, Max | −2, 1 | −2, 0 | −2, 1 | −2, 0 | −2, 1 | −2, 1 |

Change in Inflammation

| | | | | | | |
|---|---|---|---|---|---|---|
| n | 47 | 14 | 54 | 27 | 46 | 141 |
| Mean (SD) | −0.3 (0.60) | −0.5 (0.65) | −0.6 (0.69) | −0.7 (0.62) | −0.5 (0.62) | −0.6 (0.65) |
| Median | 0.0 | −1.0 | −1.0 | −1.0 | 0.0 | −1.0 |
| Min, Max | −1, 1 | −1, 1 | −2, 1 | −2, 0 | −2, 1 | −2, 1 |

Abbreviations: Max = Maximum, Min = Minimum, NAFLD = nonalcoholic fatty liver disease; NAS = nonalcoholic steatohepatitis activity score; QD = daily; QOD = every other day

TABLE 14

| | | | | | |
|---|---|---|---|---|---|
| Placebo QD | 1.0 mg QD | 2.5 mg QD | 5.0 mg QOD | 10.0 mg QOD | All VK2809 |
| (N = 62) | (N = 17) | (N = 59) | (N = 36) | (N = 57) | (N = 169) |

Resolution of steatohepatitis with no fibrosis worsening ¹

| | | | | | | |
|---|---|---|---|---|---|---|
| Response Rate % | 29.3 | 71.4 | 65.4 | 63.0 | 75.0 | 68.6 |
| 95% CI ² | 17.61, 44.48 | 45.35, 88.28 | 51.80, 76.85 | 44.23, 78.47 | 60.56, 85.43 | 60.42, 75.59 |
| p-value ³ | NA | 0.0215 | 0.0023 | 0.0091 | 0.0001 | <0.0001 |

≥1-stage Fibrosis improvement with no steatohepatitis worsening ¹

| | | | | | | |
|---|---|---|---|---|---|---|
| Response Rate % | 34.1 | 57.1 | 44.2 | 51.9 | 56.8 | 51.1 |
| 95% CI ² | 21.56, 49.45 | 32.59, 78.62 | 31.60, 57.66 | 33.99, 69.26 | 42.22, 70.32 | 42.81, 59.32 |
| p-value ³ | | 0.1543 | 0.4414 | 0.0304 | 0.0497 | 0.0308 |

≥2-point NAS improvement with no fibrosis worsening ¹

| | | | | | | |
|---|---|---|---|---|---|---|
| Response Rate % | | 64.3 | 71.2 | 70.4 | 86. | 75.2 |
| 95% CI ² | 34.1 | 38.76, 83.66 | 57.73, 81.67 | 51.52, 84.15 | 73.29, 93.60 | 67.33, 81.66 |
| p-value ³ | 21.56, 49.45 | 0.0532 | 0.0035 | 0.0016 | <0.0001 | <0.0001 |

Resolution of steatohepatitis and improvement in fibrosis ^{1,2}

| | | | | | | |
|---|---|---|---|---|---|---|
| Response Rate % | 19.5 | 50.0 | 40.4 | 40.7 | 47.7 | 43.8 |
| 95% CI ² | 10.23, 34.01 | 26.80, 73.20 | 28.16, 53.93 | 24.51. 59.27 | 33.75, 62.06 | 35.77. 52.16 |
| p-value ³ | | 0.0856 | 0.0508 | 0.0206 | 0.0115 | 0.0032 |

Abbreviations: CI = confidence interval; MRI = magnetic resonance imaging; NA = not applicable; NAS = non-alcoholic steatohepatitis activity score; NASH = nonalcoholic steatohepatitis; QD = daily; QOD = every other day
¹ Resolution of steatohepatitis is defined as a NAS score of 0-1 for inflammation, 0 for ballooning, and any value for steatosis. No worsening of liver fibrosis is defined as fibrosis score at Week 52 is less than or equal to baseline score.
² Wilson confidence interval for response rate.
³ Cochran-Mantel-Haenszel test controlling for stratification factors.

TABLE 15

| | Placebo QD | 1.0 mg QD | 2.5 mg QD | 5.0 mg QOD | 10.0 mg QOD | All VK2809 |
|---|---|---|---|---|---|---|
| | (N = 62) | (N = 17) | (N = 59) | (N = 36) | (N = 57) | (N = 169) |
| No. of Evaluable Participants | 57 | 17 | 59 | 36 | 56 | 168 |

Resolution of steatohepatitis with no fibrosis worsening ¹

| | | | | | | |
|---|---|---|---|---|---|---|
| Response Rate % | 21.1 | 58.8 | 57.6 | 47.2 | 58.9 | 56.0 |
| 95% CI ² | 12.47, 33.29 | 36.01, 78.39 | 44.93, 69.39 | 31.99, 62.99 | 45.88, 70.83 | 48.40, 63.24 |
| p-value ³ | NA | 0.0406 | 0.0002 | 0.0256 | 0.0001 | <0.0001 |

≥1-stage Fibrosis improvement with no steatohepatitis worsening ¹

| | | | | | | |
|---|---|---|---|---|---|---|
| Response Rate % | 24.6 | 47.1 | 39.0 | 38.9 | 44.6 | 41.7 |
| 95% CI ² | 15.23, 37.10 | 26.17, 69.04 | 27.58, 51.73 | 24.78, 55.14 | 32.39, 57.59 | 34.48, 49.23 |
| p-value ³ | NA | 0.2615 | 0.0850 | 0.0979 | 0.0159 | 0.0101 |

≥2-point NAS improvement with no fibrosis worsening ¹

| | | | | | | |
|---|---|---|---|---|---|---|
| Response Rate % | 24.6 | 52.9 | 62.7 | 52.8 | 67.9 | 61.3 |
| 95% CI ² | 15.23, 37.10 | 30.96, 73.83 | 49.95, 73.92 | 37.01, 68.01 | 54.82, 78.60 | 53.77, 68.34 |
| p-value ³ | NA | 0.0807 | 0.0003 | 0.0082 | <0.0001 | <0.0001 |

Resolution of steatohepatitis and improvement in fibrosis ^{1,2}

| | | | | | | |
|---|---|---|---|---|---|---|
| Response Rate % | 14.0 | 41.2 | 35.6 | 30.6 | 37.5 | 35.7 |
| 95% CI ² | 7.29, 25.32 | 21.61, 63.99 | 24.60, 48.34 | 18.00, 46.86 | 26.01, 50.59 | 28.86, 43.20 |
| p-value ³ | NA | 0.1302 | 0.0072 | 0.0440 | 0.0034 | 0.0009 |

Abbreviations: CI = confidence interval; MRI = magnetic resonance imaging; NA = not applicable; NAS = non-alcoholic steatohepatitis activity score; NASH = non-alcoholic steatohepatitis; QD = daily; QOD = every other day
¹ Resolution of steatohepatitis is defined as a NAS score of 0-1 for inflammation, 0 for ballooning, and any value for steatosis. No worsening of liver fibrosis is defined as fibrosis score at Week 52 is less than or equal to baseline score.
² Wilson confidence interval for response rate.
³ Cochran-Mantel-Haenszel test controlling for stratification factors.

Selected exploratory endpoints are discussed in this clinical study report as follows:
Normalization of liver fat <5% in the FAS at Weeks 12 and 52 and PPS at Week 12;
Absolute liver fat reduction <5% in the FAS at Weeks 12 and 52 and PPS at Week 12;
≥2-point improvement in NAS at Week 52 in the FAS, with imputation, and PPS;
≥2-Point Improvement in NAS and at Least 1-stage improvement in fibrosis on NASH CRN score at Week 52 in the FAS, with imputation, and PPS;
Ad hoc analysis of ≥2-point improvement in NAS in the FAS;
Ad hoc analysis of ≥1 stage improvement in fibrosis on NASH CRN score at Week 52 in the FAS.

Normalization of liver fat is defined as hepatic fat fraction <5% as assessed by MRI PDFF. Paired liver biopsies were conducted for each participant at the screening visit versus the Week 52 (end of treatment) visit. The same pathologist read both the screening biopsies and were blinded.

The proportion of participants with normalization of liver fat at week 12 and week 52 is presented in Table 16. The absolute reduction in liver fat ≥5% at week 12 and week 52 is presented in Table 17.

The normalization of liver fat response rate was 12.7% (CI: 8.43, 18.57) at Week 12 and 17.9% (CI: 12.54, 24.98) at Week 52 in the VK2809 arms. The response rate was 0 at Week 12 and 2.1% (CI: 0.37, 10.90) at Week 52 in the placebo arm. The response rate for the absolute reduction of liver fat ≥5% was 73.5% (CI: 66.30, 79.62; p<0.0001) at Week 12 and 76.6% (CI: 69.03, 82.71; p<0.0001) at Week 52 in the VK2809 arms. The response rate was versus 15.3% (CI: 8.24, 26.52) at Week 12 and 27.1% at Week 52 (CI: 16.57, 41.00) in the placebo arm.

TABLE 16

| | | | | | |
|---|---|---|---|---|---|
| Placebo QD | 1.0 mg QD | 2.5 mg QD | 5.0 mg QOD | 10.0 mg QOD | All VK2809 |
| (N = 62) | (N = 17) | (N = 59) | (N = 36) | (N = 57) | (N = 169) |

Week 12

| | | | | | | |
|---|---|---|---|---|---|---|
| Number of Evaluable Subjects | 59 | 17 | 59 | 36 | 54 | 166 |

Subjects with Normalization of Liver Fat [1]

| | | | | | | |
|---|---|---|---|---|---|---|
| Yes | 0 | 1 | 9 | 3 | 8 | 21 |
| No | 59 | 16 | 50 | 33 | 46 | 145 |
| Response Rate (%) | 0.0 | 5.9 | 15.3 | 8.3 | 14.8 | 12.7 |
| 95% CI [2] | 0.00, 6.11 | 1.05, 26.98 | 8.24, 26.52 | 2.87, 21.83 | 7.70, 26.60 | 8.43, 18.57 |
| Odds Ratio (VK2809 to Placebo) [3] | | NC | NC | NC | NC | NC |
| 95% CI [3] | | NC, NC | NC, NC | NC, NC | NC, NC | NC, NC |
| p-value [3] | | 0.2733 | 0.0041 | 0.2067 | 0.0013 | 0.0041 |

Week 52

| | | | | | | |
|---|---|---|---|---|---|---|
| Number of Evaluable Subjects | 48 | 14 | 55 | 27 | 49 | 145 |

Subjects with Normalization of Liver Fat [1]

| | | | | | | |
|---|---|---|---|---|---|---|
| Yes | 1 | 1 | 9 | 4 | 12 | 26 |
| No | 47 | 13 | 46 | 23 | 37 | 119 |
| Response Rate (%) | 2.1 | 7.1 | 16.4 | 14.8 | 24.5 | 17.9 |
| 95% CI [2] | 0.37, 10.90 | 1.27, 31.47 | 8.86, 28.26 | 5.92, 32.48 | 14.60, 38.09 | 12.54, 24.98 |
| Odds Ratio (VK2809 to Placebo) [3] | | NC | 7.9 | NC | NC | 14.7 |
| 95% CI [3] | | NC, NC | 1.04, 60.29 | NC, NC | NC, NC | 1.41, 152.77 |
| p-value [3] | | 0.2207 | 0.0182 | 0.1090 | 0.0003 | 0.0070 |

Abbreviations: CI = Confidence Interval, MRI-PDFF = Magnetic Resonance Imaging-Proton Density Fat Fraction, NC = Not Calculated; QD = daily; QOD = every other day
[1] Normalization of liver fat is defined as hepatic fat fraction <5% as assessed by MRI-PDFF.
[2] Wilson confidence interval for response rate.
[3] Cochran-Mantel-Haenszel test controlling for stratification factors.

TABLE 17

| | | | | | |
|---|---|---|---|---|---|
| Placebo QD | 1.0 mg QD | 2.5 mg QD | 5.0 mg QOD | 10.0 mg QOD | All VK2809 |
| (N = 62) | (N = 17) | (N = 59) | (N = 36) | (N = 57) | (N = 169) |

Week 12

| | | | | | | |
|---|---|---|---|---|---|---|
| Number of Evaluable Subjects | 59 | 17 | 59 | 36 | 54 | 166 |

Subjects with Absolute Reduction of Liver Fat ≥5% at Week 12 [1]

| | | | | | | |
|---|---|---|---|---|---|---|
| Yes | 9 | 10 | 43 | 24 | 45 | 122 |
| No | 50 | 7 | 16 | 12 | 9 | 44 |
| Response Rate (%) | 15.3 | 58.8 | 72.9 | 66.7 | 83.3 | 73.5 |
| 95% CI [2] | 8.24, 26.52 | 36.01, 78.39 | 60.40, 82.56 | 50.33, 79.79 | 71.26, 90.98 | 66.30, 79.62 |
| Odds Ratio (VK2809 to Placebo) [3] | | 5.7 | 11.8 | 6.6 | 54.9 | 15.9 |
| 95% CI [3] | | 1.43, 23.05 | 4.44, 31.29 | 2.17, 20.33 | 12.31, 244.44 | 6.87, 36.83 |
| p-value [3] | | 0.0124 | <0.0001 | 0.0005 | <0.0001 | <0.0001 |

Week 52

| | | | | | | |
|---|---|---|---|---|---|---|
| Number of Evaluable Subjects | 48 | 14 | 55 | 27 | 49 | 145 |

Subjects with Absolute Reduction of Liver Fat ≥5% at Week 52 [1]

| | | | | | | |
|---|---|---|---|---|---|---|
| Yes | 13 | 8 | 41 | 19 | 43 | 111 |
| No | 35 | 6 | 14 | 8 | 6 | 34 |
| Response Rate (%) | 27.1 | 57.1 | 74.5 | 70.4 | 87.8 | 76.6 |
| 95% CI [2] | 16.57, 41.00 | 32.59, 78.62 | 61.70, 84.19 | 51.52, 84.15 | 75.76, 94.27 | 69.03, 82.71 |
| Odds Ratio (VK2809 to Placebo) [3] | | 2.1 | 6.0 | 4.5 | 21.1 | 7.6 |
| 95% CI [3] | | 0.57, 7.77 | 2.42, 14.69 | 1.42, 14.46 | 5.92, 75.07 | 3.57, 16.19 |
| p-value [3] | | 0.2281 | <0.0001 | 0.0072 | <0.0001 | <0.0001 |

Abbreviations: CI = Confidence Interval, MRI-PDFF = Magnetic Resonance Imaging-Proton Density Fat Fraction; QD = daily; QOD = every other day
[1] At least 5% decrease from baseline in Hepatic fat fraction as assessed by MRI-PDFF.
[2] Wilson confidence interval for response rate.
[3] Cochran-Mantel-Haenszel test controlling for stratification factors.

The NASH/MASH parameters and individual components are shown in Table 18. Participants in all treatment arms recorded improvements in NASH/MASH; albeit the improvements were greater in the VK2809 treatment arms for each component. The greatest improvements in the VK2809 treatment arms were observed in NASH/MASH, steatosis, and hepatocyte ballooning. Comparisons in treatment arms at Week 52 are as follows:
NASH/MASH Improvement: VK2809 treatment arms 55.8% vs. placebo arm 30.0%;
Steatosis Improvement: VK2809 treatment arms 57.9% vs. placebo arm 46.7%;
Hepatocyte ballooning Improvement: VK2809 treatment arms 56.9% vs. placebo arm 34.0%;
Inflammation Improvement: VK2809 treatment arms 44.2% vs. placebo arm 22.0%;
Fibrosis Improvement: VK2809 treatment arms 36.5% vs. placebo arm 32.0%.

The response rate for participants with at least a 2-point improvement in NAS and 1-stage improvement liver fibrosis at Week 52 is presented in Table 19. The response rate for ≥2 point or greater improvement was 84.2% in the VK2809 arms versus 33.3% in the placebo arm. The response rate increased with increasing VK2809 dose. Statistically significant reductions were observed for all VK2809 treatment arms (p≤0.0448). With the addition of a 1-stage improvement in liver fibrosis, the response rate at Week 52 was 48.2% in the VK2809 treatment arms versus 24.4% in the placebo arm. The response rate increased with increasing VK2809 dose. Statistically significant reductions were observed for the two higher VK2809 treatment arms (p≤0.0273).

Ad hoc analyses of these data in which participants with a missing liver biopsy at Week 52 were considered non-responders are presented in Table 20. The response rate for ≥2-point improvement was 69.6% in the VK2809 arms versus 24.6% in the placebo arm. Statistically significant reductions were observed for all VK2809 treatment arms (p≤0.0011). With the addition of a 1-stage improvement in liver fibrosis, the response rate at Week 52 was 39.3% in the VK2809 treatment arms versus 17.5% in the placebo arm. Statistically significant reductions were observed for the 2 VK2809 treatment arms (p≤0.0105), the 2.5 mg QD and the 10.0 mg QOD dose.

TABLE 18

| | | | | | | |
|---|---|---|---|---|---|---|
| Parameter | Placebo QD | 1.0 mg QD | 2.5 mg QD | 5.0 mg QOD | 10.0 mg QOD | All VK2809 |
| Category | (N = 62) | (N = 17) | (N = 59) | (N = 36) | (N = 57) | (N = 231) |

Week 52 End of Treatment
NASH

| n | 50 | | 15 | 54 | | 30 | | 48 | | 197 | |
|---|---|---|---|---|---|---|---|---|---|---|---|
| Yes | 30 | (60.0) | 6 (40.0) | 18 | (33.3) | 9 | (30.0) | 8 | (16.7) | 71 | (36.0) |
| No | 3 | (6.0) | 2 (13.3) | 19 | (35.2) | 6 | (20.0) | 17 | (35.4) | 47 | (23.9) |
| Borderline | 16 | (32.0) | 7 (46.7) | 15 | (27.8) | 15 | (50.0) | 23 | (47.9) | 76 | (38.6) |
| Not Evaluable | 1 | (2.0) | 0 | 2 | (3.7) | 0 | | 0 | | 3 | (1.5) |
| Better | 15 | (30.0) | 9 (60.0) | 34 | (63.0) | 19 | (63.3) | 33 | (68.8) | 110 | (55.8) |
| Same | 27 | (54.0) | 4 (26.7) | 12 | (22.2) | 9 | (30.0) | 10 | (20.8) | 62 | (31.5) |
| Worse | 8 | (16.0) | 2 (13.3) | 7 | (13.0) | 2 | (6.7) | 5 | (10.4) | 24 | (12.2) |

Steatosis

| | | | | | | | | | | | |
|---|---|---|---|---|---|---|---|---|---|---|---|
| Better | 12 | (24.0) | 7 (46.7) | 38 | (70.4) | 18 | (60.0) | 39 | (81.3) | 114 | (57.9) |
| Same | 32 | (64.0) | 6 (40.0) | 10 | (18.5) | 7 | (23.3) | 4 | (8.3) | 59 | (29.9) |
| Worse | 6 | (12.0) | 2 (13.3) | 5 | (9.3) | 5 | (16.7) | 5 | (10.4) | 23 | (11.7) |

Hepatocyte Ballooning

| | | | | | | | | | | | |
|---|---|---|---|---|---|---|---|---|---|---|---|
| Better | 17 | (34.0) | 9 (60.0) | 36 | (66.7) | 19 | (63.3) | 31 | (64.6) | 112 | (56.9) |
| Same | 26 | (52.0) | 2 (13.3) | 12 | (22.2) | 9 | (30.0) | 12 | (25.0) | 61 | (31.0) |
| Worse | 6 | (12.0) | 4 (26.7) | 5 | (9.3) | 2 | (6.7) | 5 | (10.4) | 22 | (11.2) |

Inflammation

| | | | | | | | | | | | |
|---|---|---|---|---|---|---|---|---|---|---|---|
| Better | 11 | (22.0) | 9 (60.0) | 30 | (55.6) | 11 | (36.7) | 26 | (54.2) | 87 | (44.2) |
| Same | 34 | (68.0) | 3 (20.0) | 18 | (33.3) | 17 | (56.7) | 18 | (37.5) | 90 | (45.7) |
| Worse | 5 | (10.0) | 3 (20.0) | 5 | (9.3) | 2 | (6.7) | 4 | (8.3) | 19 | (9.6) |

Fibrosis

| | | | | | | | | | | | |
|---|---|---|---|---|---|---|---|---|---|---|---|
| Better | 16 | (32.0) | 7 (46.7) | 22 | (40.7) | 10 | (33.3) | 17 | (35.4) | 72 | (36.5) |
| Same | 25 | (50.0) | 6 (40.0) | 23 | (42.6) | 18 | (60.0) | 22 | (45.8) | 94 | (47.7) |
| Worse | 6 | (12.0) | 2 (13.3) | 6 | (11.1) | 2 | (6.7) | 7 | (14.6) | 23 | (11.7) |

Note:
Percentages are based on the number of subjects with assessments.
Abbreviations: NASH = Non-alcoholic Steatohepatitis; QD = daily; QOD = every other day

TABLE 19

| | Placebo QD | 1.0 mg QD | 2.5 mg QD | 5.0 mg QOD | 10.0 mg QOD | All VK2809 |
|---|---|---|---|---|---|---|
| | (N = 62) | (N = 17) | (N = 59) | (N = 36) | (N = 57) | (N = 169) |
| Number of Evaluable Subjects | 42 | 14 | 53 | 27 | 45 | 139 |

Subjects with >=2-Point Improvement in NAS at Week 52 [1]

| | | | | | | |
|---|---|---|---|---|---|---|
| Yes | 14 | 9 | 45 | 23 | 40 | 117 |
| No | 28 | 5 | 8 | 4 | 5 | 22 |
| Response Rate (%) | 33.3 | 64.3 | 84.9 | 85.2 | 88.9 | 84.2 |
| 95% CI [2] | 21.01, 48.45 | 38.76, 83.66 | 72.95, 92.15 | 67.52, 94.08 | 76.50, 95.16 | 77.20, 89.31 |
| Odds Ratio (VK2809 to Placebo) [3] | | 4.5 | 10.4 | 13.2 | 20.6 | 11.8 |
| 95% CI [3] | | 1.04, 19.20 | 3.50, 30.67 | 2.89, 60.38 | 4.60, 92.18 | 4.95, 28.35 |
| p-value [3] | | 0.0448 | <0.0001 | 0.0002 | <0.0001 | <0.0001 |

Subjects with >=2-Point Improvement in NAS and at Least 1-Stage Improvement in Liver Fibrosis at Week 52 [1]

| | | | | | | |
|---|---|---|---|---|---|---|
| Number of Evaluable Subjects | 41 | 14 | 52 | 27 | 44 | 137 |
| Yes | 10 | 6 | 23 | 12 | 25 | 66 |
| No | 31 | 8 | 29 | 15 | 19 | 71 |
| Response Rate (%) | 24.4 | 42.9 | 44.2 | 44.4 | 56.8 | 48.2 |
| 95% CI [2] | 13.83, 39.34 | 21.38, 67.41 | 31.60, 57.66 | 27.59, 62.69 | 42.22, 70.32 | 39.97, 56.48 |
| Odds Ratio (VK2809 to Placebo) [3] | | 2.1 | 2.3 | 4.4 | 4.6 | 3.3 |
| 95% CI [3] | | 0.52, 8.61 | 0.92, 5.63 | 1.12, 17.54 | 1.58, 13.13 | 1.45, 7.35 |
| p-value [3] | | 0.3023 | 0.0787 | 0.0273 | 0.0037 | 0.0034 |

Abbreviations: CI = Confidence Interval, NAS = NASH Activity Score, NASH = nonalcoholic steatohepatitis; QD = daily; QOD = every other day
[1] At least 2-point improvement in NAS from baseline and at least 1-stage improvement in liver fibrosis on NASH CRN Score at Week 52.
[2] Wilson confidence interval for response rate.
[3] Cochran-Mantel-Haenszel test controlling for stratification factors.

TABLE 20

| | Placebo QD | 1.0 mg QD | 2.5 mg QD | 5.0 mg QOD | 10.0 mg QOD | All VK2809 |
|---|---|---|---|---|---|---|
| | (N = 62) | (N = 17) | (N = 59) | (N = 36) | (N = 57) | (N = 169) |
| Number of Evaluable Subjects | 57 | 17 | 59 | 36 | 56 | 168 |

Subjects with ≥2-Point Improvement in NAS at Week 52 [1]

| | | | | | | |
|---|---|---|---|---|---|---|
| Yes | 14 | 9 | 45 | 23 | 40 | 117 |
| No | 43 | 8 | 14 | 13 | 16 | 51 |
| Response Rate (%) | 24.6 | 52.9 | 76.3 | 63.9 | 71.4 | 69.6 |
| 95% CI [2] | 15.23, 37.10 | 30.96, 73.83 | 64.03, 85.31 | 47.58, 77.52 | 58.52, 81.58 | 62.32, 76.09 |
| Odds Ratio (VK2809 to Placebo) [3] | | 3.2 | 8.7 | 5.9 | 9.6 | 7.6 |
| 95% CI [3] | | 0.88, 11.87 | 3.59, 21.07 | 1.96, 17.49 | 3.62, 25.27 | 3.67, 15.70 |
| p-value [3] | | 0.0807 | <0.0001 | 0.0011 | <0.0001 | <0.0001 |

Subjects with ≥2-Point Improvement in NAS and ≥1-Stage Improvement in Liver Fibrosis at Week 52 [1]

| | | | | | | |
|---|---|---|---|---|---|---|
| Number of Evaluable Subjects | 57 | 17 | 59 | 36 | 56 | 168 |
| Yes | 10 | 6 | 23 | 12 | 25 | 66 |
| No | 47 | 11 | 36 | 24 | 31 | 102 |
| Response Rate (%) | 17.5 | 35.3 | 39.0 | 33.3 | 44.6 | 39.3 |
| 95% CI [2] | 9.82, 29.37 | 17.31, 58.70 | 27.58, 51.73 | 20.21, 49.67 | 32.39, 57.59 | 32.22, 46.83 |
| Odds Ratio (VK2809 to Placebo) [3] | | 1.9 | 3.0 | 2.9 | 4.8 | 3.5 |
| 95% CI [3] | | 0.48, 7.19 | 1.32, 7.05 | 0.90, 9.40 | 1.85, 12.58 | 1.63, 7.50 |
| p-value [3] | | 0.3949 | 0.0105 | 0.0655 | 0.0010 | 0.0010 |

Note:
Participants with a missing liver biopsy at Week 52 are considered non-responders
Abbreviations: CI = Confidence Interval, NAS = NASH Activity Score, NASH = non-alcoholic steatohepatitis; QD = daily; QOD = every other day.
[1] At least 2-point improvement in NAS from baseline and at least 1-stage improvement in liver fibrosis on NASH CRN Score at Week 52.
[2] Wilson confidence interval for response rate.
[3] Cochran-Mantel-Haenszel test controlling for stratification factors.

Selected safety analyses are discussed in this clinical study report as follows:
NASH/MASH Biomarker results, change from baseline and percent change LSM and LSM difference from baseline at Week 52;
Liver profile change from baseline and percent change LSM and LSM difference from baseline at Week 52;
Thyroid profile change from baseline and percent change LSM and LSM difference from baseline at Week 52.

Biomarkers are tools to determine whether the NASH/MASH diagnosis is improving or worsening with VK2809 treatment. Reduction in biomarkers is indicative or NASH/MASH improvement whereas increases indicate worsening of NASH/MASH. The Enhanced Liver Fibrosis Score (ELF i.e., Fibrotest) comprises 3 components assessed in this study: hyaluronic acid, and TIMP1/creatinine. Other NASH/MASH markers include NIS4 and Pro-C3.

Changes in markers of hepatic fibrosis and steatosis from baseline to Week 52 are shown in Table 21. Overall, reductions were more prevalent than increases in NASH/MASH biomarkers. Moreover, the reductions were greater in the VK2809 treatment arms versus the placebo arm. However, the VK2809 5.0 mg QOD treatment arm exhibited more increases indictive of worsening NASH/MASH. Reductions in all treatment arms were noted for Pro-C3 absolute and relative mean, median, LSM, and LSM difference from baseline to Week 52.

ELF scores (mean, median, LSM [absolute change], LSM difference [relative change]) were reduced at Week 52 but greater reduction was noted for participants in the VK2809 treatment arms versus the placebo arm. Moreover, in the VK2809 5.0 mg QOD arm ELF scores were increased for mean and median absolute and relative change from baseline to Week 52, and the corresponding LSM (absolute change), and LSM difference (relative change).

The P1INP biomarker, a component of ELF scores, was reduced in all treatment arms including placebo, except for the VK2809 5.0 mg QOD arm. In that treatment arm, mean and mean absolute and relative change from baseline to LSM difference increased from baseline to Week 52.

The TIMP 1/creatinine biomarker, a component of ELF scores were reduced in all treatment arms including placebo, except the VK2809 1.0 mg QD arm. In that treatment arm, increases in median absolute and relative change from baseline were noted to Week 52. The absolute and relative LSM difference in the 2.5 mg QD VK2809 arm with a p-value=0.0072.

Hyaluronic acid results were variable with increases in absolute mean, median, and LSM, and LSM difference (VK2809 5.0 mg QOD only) in the placebo and VK2809 5.0 mg QOD treatment arm and reductions for all other treatment arms. Increases were also noted for relative mean and LSM in all treatment arms, and LSM difference for VK2809 5.0 mg QOD. Reductions were noted for all other parameters.

Reductions in NIS4 were noted for absolute mean, median, LSM, and LSM mean differences for all treatment arms. Most relative mean, median, LSM, and LSM mean differences (p=0.0096 for 2.5 mg VK2809; p-0.0159 for VK2809 10.0 mg QOD) were noted for all treatment arms except the mean for placebo and LSM for the placebo and VK2809 5.0 mg QOD treatment arms.

The change and percent change in liver profile from baseline to Week 52 is shown in Table 22. Absolute and relative reductions in ALT and AST were noted in all treatment arms but changes were greater with VK2809 treatment:
ALT absolute LSM change for VK2809 arms (LSM≥16.20; LSM difference ≥2.45);
ALT relative LSM change for VK2809 arms (LSM≥18.41; LSM difference ≥5.73; p<0.0431 10.0 mg QOD VK2809 arm);
AST absolute LSM change for VK2809 arms (LSM≥11.42; LSM difference ≥7.57; p≤0.0477 1.0 mg QD and 10.0 mg QOD VK2809 arm);
AST relative LSM change for VK2809 arms (LSM≥18.51; LSM difference ≥16.81; p<0.0418 1.0 mg QD VK2809 arm).

Absolute and relative reductions in GGT were noted in the VK2809 arms but not the placebo arm:
GTT absolute LSM change for VK2809 arms (LSM≥19.19; LSM difference ≥45.00; p <0.0314 all VK2809 arm);
GTT relative LSM change for VK2809 arms (LSM≥4.7; LSM difference ≥12.7;
p≤0.0211 in all VK2809 arms except 5.0 mg QOD).

Reductions from baseline in albumin, prothrombin time, prothrombin international normalized ratio were noted in all treatment arms. Increases from baseline were noted for direct bilirubin with greater changes in the VK2809 arms. Variable changes for alkaline phosphatase, bilirubin, and indirect bilirubin.

The change and percent change in thyroid profile from baseline to Week 52 is shown in Table 23. Absolute and relative LSM reductions in triiodothyronine (T3) were observed in all treatment arms except the 1.0 mg VK2809 arm. Absolute and relative LSM increases in sex hormone binding globulin (SHBG) were observed in all treatment arms except the 1.0 mg VK2809 arm. Variable absolute and relative changes in thyrotropin, thyroxine (T4).

Overall, lipid measurements of mean and median HDL and LDL cholesterol, total cholesterol, triglycerides, lipoprotein A, and apolipoprotein B improved in all treatment arms. However, the changes were more pronounced in the VK2809 treatment arms with greater differences noted at the higher VK2809 dose. The change and percent change LSM and LSM difference from baseline in lipid profile from baseline at Week 52 are as follows:
Absolute and relative increases in HDL cholesterol were observed in all treatment arms. LSM differences were notable for the 10.0 mg VK2809 treatment arm (p≤0.103);
Absolute and relative decreases in LDL cholesterol were observed in all treatment arms except relative change from baseline in the placebo arm. LSM differences were also notable in all VK2809 treatment arms (p≤0.034);
Absolute and relative decreases in cholesterol were observed in all treatment arms. LSM differences were also notable in all VK2809 treatment arms (p≤0.0205);
Absolute and relative decreases in triglycerides were observed in all VK2809 treatment arms. LSM differences were also notable VK2809 2.5 mg QD and 10.0 mg QOD arms (p≤0.0056);
Absolute and relative LSM decreases in lipoprotein A were observed in all VK2809 treatment arms but not placebo arms. LSM differences were also notable in all VK2809 treatment arms except the 1.0 mg QD arm (p≤0.0011);
Absolute and relative LSM decreases in apolipoprotein B were observed in all treatment arms. LSM differences were also notable in all VK2809 treatment arms (p≤0.0074).

TABLE 21

| | | | | | | |
|---|---|---|---|---|---|---|
| Parameter | Placebo QD | 1.0 mg QD | 2.5 mg QD | 5.0 mg QOD | 10.0 mg QOD | All VK2809 |
| Visit | (N = 65) | (N = 17) | (N = 66) | (N = 37) | (N = 61) | (N = 181) |

ELF (FibroTest)
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | −0.091 | (0.107) | −0.343 | (0.217) | −0.253 | (0.098) | 0.076 | (0.146) | −0.227 | (0.104) | Not Calculated |

| | | | | | | | | | | |
|---|---|---|---|---|---|---|---|---|---|---|
| LSM Difference SEM [2] | Not Applicable | −0.252 | (0.236) | −0.162 | (0.139) | 0.167 | (0.175) | −0.135 | (0.143) | Not Calculated |

| | | | | | | |
|---|---|---|---|---|---|---|
| p-value | Not Applicable | 0.2860 | 0.2466 | 0.3405 | 0.3447 | 0.2860 |

Percent Change from Baseline to Week 52 - EOT

| | | | | | | | | | | | |
|---|---|---|---|---|---|---|---|---|---|---|---|
| LSM (SEM [2]) | −0.96 | (1.10) | −3.49 | (2.25) | −2.35 | (1.01) | 0.82 | (1.51) | −2.16 | (1.07) | Not Calculated |

| | | | | | | | | | | |
|---|---|---|---|---|---|---|---|---|---|---|
| LSM Difference | Not Applicable | −2.521 | (2.437) | −1.386 | (1.439) | 1.780 | (1.809) | −1.191 | (1.479) | Not Calculated |
| (SEM) [2] | | | | | | | | | | |

| | | | | | | |
|---|---|---|---|---|---|---|
| p-value | Not Applicable | 0.3025 | 0.3367 | 0.3263 | 0.4216 | Not Calculated |

NIS4
Change from Baseline to Week 52 - EOT

| n | 44 | | 11 | | 51 | | 21 | | 46 | | 129 |
|---|---|---|---|---|---|---|---|---|---|---|---|
| LSM (SEM) [2] | −0.084 | (0.042) | −0.151 | (0.084) | −0.126 | (0.038) | −0.035 | (0.062) | −0.096 | (0.040) | Not Calculated |

| | | | | | | | | | | |
|---|---|---|---|---|---|---|---|---|---|---|
| LSM Difference SEM [2] | Not Applicable | −0.067 | (0.091) | −0.041 | (0.054) | 0.049 | (0.073) | −0.012 | (0.055) | Not Calculated |

| | | | | | | |
|---|---|---|---|---|---|---|
| p-value | Not Applicable | 0.4616 | 0.4427 | 0.4990 | 0.8240 | Not Calculated |

Percent Change from Baseline to Week 52 - EOT

| | | | | | | | | | | | |
|---|---|---|---|---|---|---|---|---|---|---|---|
| LSM (SEM) [2] | 20.86 | (9.69) | −7.92 | (19.70) | −12.08 | (8.82) | 0.48 | (14.40) | −10.61 | (9.29) | Not Calculated |

| | | | | | | | | | | |
|---|---|---|---|---|---|---|---|---|---|---|
| LSM Difference SEM [2] | Not Applicable | −28.779 | (21.327) | −32.940 | (12.561) | −20.380 | (16.946) | −31.473 | (12.909) | Not Calculated |

| | | | | | | |
|---|---|---|---|---|---|---|
| p-value | Not Applicable | 0.1791 | 0.0096 | 0.2309 | 0.0159 | Not Calculated |

Pro-C3
Change from Baseline to Week 52 - EOT

| n | 45 | | 12 | | 52 | | 25 | | 46 | | 135 |
|---|---|---|---|---|---|---|---|---|---|---|---|
| LSM (SEM) [2] | −20.367 | (7.529) | −32.862 | (15.015) | −36.040 | (6.779) | −34.007 | (10.776) | −32.471 | (7.242) | Not Calculated |

| | | | | | | | | | | |
|---|---|---|---|---|---|---|---|---|---|---|
| LSM Difference SEM [2] | Not Applicable | −12.495 | (16.327) | −15.673 | (9.724) | −13.640 | (12.619) | −12.104 | (9.971) | Not Calculated |

| | | | | | | |
|---|---|---|---|---|---|---|
| p-value | Not Applicable | 0.4452 | 0.1089 | 0.2813 | 0.2265 | Not Calculated |

Percent Change from Baseline to Week 52 - EOT

| | | | | | | | | | | | |
|---|---|---|---|---|---|---|---|---|---|---|---|
| LSM (SEM) [2] | −13.68 | (5.11) | −24.84 | (9.92) | −21.39 | (4.60) | −18.54 | (7.15) | −18.21 | (4.92) | Not Calculated |

| | | | | | | | | | | |
|---|---|---|---|---|---|---|---|---|---|---|
| LSM Difference SEM [2] | Not Applicable | −11.160 | (10.854) | −7.708 | (6.605) | −4.865 | (8.445) | −4.532 | (6.781) | Not Calculated |

| | | | | | | |
|---|---|---|---|---|---|---|
| p-value | Not Applicable | 0.3053 | 0.2448 | 0.5653 | 0.5048 | Not Calculated |

TIMP1/Creatinine
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | −4.725 | (7.876) | −12.117 | (15.157) | −32.556 | (7.210) | −15.288 | (10.756) | −16.511 | (7.627) | Not Calculated |

| | | | | | | | | | | |
|---|---|---|---|---|---|---|---|---|---|---|
| LSM Difference SEM [2] | Not Applicable | −7.392 | (16.484) | −27.831 | (10.242) | −10.562 | (12.742) | −11.786 | (10.556) | Not Calculated |

| | | | | | | |
|---|---|---|---|---|---|---|
| p-value [2] | Not Applicable | 0.6544 | 0.0072 | 0.4082 | 0.2657 | Not Calculated |

Percent Change from Baseline to Week 52 - EOT

| | | | | | | | | | | | |
|---|---|---|---|---|---|---|---|---|---|---|---|
| LSM (SEM) [2] | −1.63 | (2.50) | −3.21 | (4.81) | −9.88 | (2.29) | −4.74 | (3.41) | −4.46 | (2.42) | Not Calculated |

| | | | | | | | | | | |
|---|---|---|---|---|---|---|---|---|---|---|
| LSM Difference SEM [2] | Not Applicable | −1.574 | (5.231) | −8.244 | (3.250) | −3.106 | (4.044) | −2.823 | (3.350) | Not Calculated |

| | | | | | | |
|---|---|---|---|---|---|---|
| p-value [2] | Not Applicable | 0.7639 | 0.0120 | 0.4435 | 0.4004 | Not Calculated |

Hyaluronic Acid Baseline (ng/mL)
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | 10.911 | (7.854) | −6.268 | (18.242) | −4.977 | (7.304) | 12.202 | (10.690) | −4.194 | (7.608) | Not Calculated |

| | | | | | | | | | | |
|---|---|---|---|---|---|---|---|---|---|---|
| LSM Difference SEM [2] | Not Applicable | −17.179 | (19.418) | −15.889 | (10.246) | 1.291 | (12.771) | −15.105 | (10.504) | Not Calculated |

| | | | | | | |
|---|---|---|---|---|---|---|
| p-value [2] | Not Applicable | 0.3775 | 0.1228 | 0.9196 | 0.1522 | Not calculated |

Percent Change from Baseline to Week 52 - EOT

| | | | | | | | | | | | |
|---|---|---|---|---|---|---|---|---|---|---|---|
| LSM (SEM) [2] | 22.91 | (10.58) | 6.57 | (20.40) | 7.68 | (9.75) | 33.22 | (14.41) | 3.80 | (10.30) | Not Calculated |

| | | | | | | | | | | |
|---|---|---|---|---|---|---|---|---|---|---|
| LSM Difference SEM [2] | Not Applicable | −16.346 | (22.338) | −15.232 | (13.817) | 10.309 | (17.229) | −19.112 | (14.183) | Not Calculated |

| | | | | | | |
|---|---|---|---|---|---|---|
| p-value [2] | Not Applicable | 0.4653 | 0.2717 | 0.5503 | 0.1795 | Not Calculated |

Procollagen 3 N-Terminal Propeptide
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | −0.942 | (0.590) | −2.749 | (1.203) | −1.869 | (0.539) | −0.659 | (0.841) | −1.291 | (0.566) | Not Calculated |

| | | | | | | | | | | |
|---|---|---|---|---|---|---|---|---|---|---|
| LSM Difference SEM [2] | Not Applicable | −1.806 | (1.297) | −0.926 | (0.769) | 0.284 | (0.980) | −0.348 | (0.784) | Not Calculated |

| | | | | | | |
|---|---|---|---|---|---|---|
| p-value [2] | Not Applicable | 0.1654 | 0.2297 | 0.7724 | 0.6575 | Not Calculated |

Percent Change from Baseline to Week 52 - EOT

| Mean (SD) | −5.13 | (41.749) | −7.66 | (36.536) | −12.11 | (33.682) | 11.78 | (42.188) | −4.38 | (37.677) | −4.43 (37.679) |
|---|---|---|---|---|---|---|---|---|---|---|---|
| Median | −11.85 | | −15.99 | | −19.00 | | 2.10 | | −11.72 | | −12.15 |
| Min, Max | −59.4, 131.2 | | −55.7, 54.0 | | −70.3, 100.0 | | −61.1, 148.9 | | −65.6, 104.9 | | −70.3, 148.9 |
| LSM (SEM) [2] | −8.18 | (5.43) | −19.48 | (11.06) | −11.20 | (4.96) | −1.32 | (7.73) | −5.46 | (5.20) | Not Calculated |

| | | | | | | | | | | |
|---|---|---|---|---|---|---|---|---|---|---|
| LSM Difference SEM [2] | Not Applicable | −11.300 | (11.924) | −3.014 | (7.068) | 6.858 | (9.012) | 2.718 | (7.211) | Not Calculated |

| | | | | | | |
|---|---|---|---|---|---|---|
| p-value [2] | Not Applicable | 0.3446 | 0.6703 | 0.4477 | 0.7067 | Not Calculated |

Note:
Baseline is the last assessment prior to the first dose of study drug.
Abbreviations: ELF = enhanced liver fibrosis. EOT = end of treatment, NIS4 = non-invasive score-4, PIIINP = amino-terminal propeptide of type III procollagen, QD = daily, QOD = every other day, TIMP1 = tissue inhibitor of metalloproteinases 1, ANCOVA = analysis of covariance, LSM = Least Squares Mean, QD = daily, QOD = every other day
[2] Results are from an Analysis of Covariance Model (ANCOVA) with randomization stratification factors (including whether the subject was randomized before/after the interim analysis), treatment assignments, baseline value, and the interaction of baseline value with treatment as covariates. The interaction of baseline value with treatment is not retained in the model if the p-value for the interaction is greater than the 0.15-level of significance and the model reran before estimates are computed.

TABLE 22

| | | | | | | |
|---|---|---|---|---|---|---|
| Parameter | Placebo QD | 1.0 mg QD | 2.5 mg QD | 5.0 mg QOD | 10.0 mg QOD | All VK2809 |
| Visit | (N = 65) | (N = 17) | (N = 66) | (N = 37) | (N = 61) | (N = 181) |

ELF (FibroTest)
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | −0.091 | (0.107) | −0.343 | (0.217) | −0.253 | (0.098) | 0.076 | (0.146) | −0.227 | (0.104) | Not Calculated |

| | | | | | | | | | | |
|---|---|---|---|---|---|---|---|---|---|---|
| LSM Difference SEM [2] | Not Applicable | −0.252 | (0.236) | −0.162 | (0.139) | 0.167 | (0.175) | −0.135 | (0.143) | Not Calculated |

| | | | | | | |
|---|---|---|---|---|---|---|
| p-value | Not Applicable | 0.2860 | 0.2466 | 0.3405 | 0.3447 | 0.2860 |

Percent Change from Baseline to Week 52 - EOT

| | | | | | | | | | | | |
|---|---|---|---|---|---|---|---|---|---|---|---|
| LSM (SEM [2]) | −0.96 | (1.10) | −3.49 | (2.25) | −2.35 | (1.01) | 0.82 | (1.51) | −2.16 | (1.07) | Not Calculated |

| | | | | | | | | | | |
|---|---|---|---|---|---|---|---|---|---|---|
| LSM Difference | Not Applicable | −2.521 | (2.437) | −1.386 | (1.439) | 1.780 | (1.809) | −1.191 | (1.479) | Not Calculated |
| (SEM) [2] | | | | | | | | | | |

| | | | | | | |
|---|---|---|---|---|---|---|
| p-value | Not Applicable | 0.3025 | 0.3367 | 0.3263 | 0.4216 | Not Calculated |

NIS4
Change from Baseline to Week 52 - EOT

| n | 44 | | 11 | | 51 | | 21 | | 46 | | 129 |
|---|---|---|---|---|---|---|---|---|---|---|---|
| LSM (SEM) [2] | −0.084 | (0.042) | −0.151 | (0.084) | −0.126 | (0.038) | −0.035 | (0.062) | −0.096 | (0.040) | Not Calculated |

| | | | | | | | | | | |
|---|---|---|---|---|---|---|---|---|---|---|
| LSM Difference SEM [2] | Not Applicable | −0.067 | (0.091) | −0.041 | (0.054) | 0.049 | (0.073) | −0.012 | (0.055) | Not Calculated |

| | | | | | | |
|---|---|---|---|---|---|---|
| p-value | Not Applicable | 0.4616 | 0.4427 | 0.4990 | 0.8240 | Not Calculated |

Percent Change from Baseline to Week 52 - EOT

| | | | | | | | | | | | |
|---|---|---|---|---|---|---|---|---|---|---|---|
| LSM (SEM) [2] | 20.86 | (9.69) | −7.92 | (19.70) | −12.08 | (8.82) | 0.48 | (14.40) | −10.61 | (9.29) | Not Calculated |

| | | | | | | | | | | |
|---|---|---|---|---|---|---|---|---|---|---|
| LSM Difference SEM [2] | Not Applicable | −28.779 | (21.327) | −32.940 | (12.561) | −20.380 | (16.946) | −31.473 | (12.909) | Not Calculated |

| | | | | | | |
|---|---|---|---|---|---|---|
| p-value | Not Applicable | 0.1791 | 0.0096 | 0.2309 | 0.0159 | Not Calculated |

Pro-C3
Change from Baseline to Week 52 - EOT

| n | 45 | | 12 | | 52 | | 25 | | 46 | | 135 |
|---|---|---|---|---|---|---|---|---|---|---|---|
| LSM (SEM) [2] | −20.367 | (7.529) | −32.862 | (15.015) | −36.040 | (6.779) | −34.007 | (10.776) | −32.471 | (7.242) | Not Calculated |

| | | | | | | | | | | |
|---|---|---|---|---|---|---|---|---|---|---|
| LSM Difference SEM [2] | Not Applicable | −12.495 | (16.327) | −15.673 | (9.724) | −13.640 | (12.619) | −12.104 | (9.971) | Not Calculated |

| | | | | | | |
|---|---|---|---|---|---|---|
| p-value | Not Applicable | 0.4452 | 0.1089 | 0.2813 | 0.2265 | Not Calculated |

Percent Change from Baseline to Week 52 - EOT

| | | | | | | | | | | | |
|---|---|---|---|---|---|---|---|---|---|---|---|
| LSM (SEM) [2] | −13.68 | (5.11) | −24.84 | (9.92) | −21.39 | (4.60) | −18.54 | (7.15) | −18.21 | (4.92) | Not Calculated |

| | | | | | | | | | | |
|---|---|---|---|---|---|---|---|---|---|---|
| LSM Difference SEM [2] | Not Applicable | −11.160 | (10.854) | −7.708 | (6.605) | −4.865 | (8.445) | −4.532 | (6.781) | Not Calculated |

| | | | | | | |
|---|---|---|---|---|---|---|
| p-value | Not Applicable | 0.3053 | 0.2448 | 0.5653 | 0.5048 | Not Calculated |

TIMP1/Creatinine
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | −4.725 | (7.876) | −12.117 | (15.157) | −32.556 | (7.210) | −15.288 | (10.756) | −16.511 | (7.627) | Not Calculated |

| | | | | | | | | | | |
|---|---|---|---|---|---|---|---|---|---|---|
| LSM Difference SEM [2] | Not Applicable | −7.392 | (16.484) | −27.831 | (10.242) | −10.562 | (12.742) | −11.786 | (10.556) | Not Calculated |

| | | | | | | |
|---|---|---|---|---|---|---|
| p-value [2] | Not Applicable | 0.6544 | 0.0072 | 0.4082 | 0.2657 | Not Calculated |

Percent Change from Baseline to Week 52 - EOT

| | | | | | | | | | | | |
|---|---|---|---|---|---|---|---|---|---|---|---|
| LSM (SEM) [2] | −1.63 | (2.50) | −3.21 | (4.81) | −9.88 | (2.29) | −4.74 | (3.41) | −4.46 | (2.42) | Not Calculated |

| | | | | | | | | | | |
|---|---|---|---|---|---|---|---|---|---|---|
| LSM Difference SEM [2] | Not Applicable | −1.574 | (5.231) | −8.244 | (3.250) | −3.106 | (4.044) | −2.823 | (3.350) | Not Calculated |

| | | | | | | |
|---|---|---|---|---|---|---|
| p-value [2] | Not Applicable | 0.7639 | 0.0120 | 0.4435 | 0.4004 | Not Calculated |

Hyaluronic Acid Baseline (ng/mL)
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | 10.911 | (7.854) | −6.268 | (18.242) | −4.977 | (7.304) | 12.202 | (10.690) | −4.194 | (7.608) | Not Calculated |

| | | | | | | | | | | |
|---|---|---|---|---|---|---|---|---|---|---|
| SEM [2] | Not Applicable | −17.179 | (19.418) | −15.889 | (10.246) | 1.291 | (12.771) | −15.105 | (10.504) | Not Calculated |

| | | | | | | |
|---|---|---|---|---|---|---|
| LSM Difference | Not Applicable | 0.3775 | 0.1228 | 0.9196 | 0.1522 | Not calculated |
| p-value [2] | | | | | | |

Percent Change from Baseline to Week 52 - EOT

| | | | | | | | | | | | |
|---|---|---|---|---|---|---|---|---|---|---|---|
| LSM (SEM) [2] | 22.91 | (10.58) | 6.57 | (20.40) | 7.68 | (9.75) | 33.22 | (14.41) | 3.80 | (10.30) | Not Calculated |

| | | | | | | | | | | |
|---|---|---|---|---|---|---|---|---|---|---|
| LSM Difference SEM [2] | Not Applicable | −16.346 | (22.338) | −15.232 | (13.817) | 10.309 | (17.229) | −19.112 | (14.183) | Not Calculated |

| | | | | | | |
|---|---|---|---|---|---|---|
| p-value [2] | Not Applicable | 0.4653 | 0.2717 | 0.5503 | 0.1795 | Not Calculated |

Procollagen 3 N-Terminal Propeptide
Change from Baseline to Week 52 - EOT

| n | 48 | | 13 | | 54 | | 28 | | 48 | | 143 |
|---|---|---|---|---|---|---|---|---|---|---|---|
| LSM (SEM) [2] | −0.942 | (0.590) | −2.749 | (1.203) | −1.869 | (0.539) | −0.659 | (0.841) | −1.291 | (0.566) | Not Calculated |

| | | | | | | | | | | |
|---|---|---|---|---|---|---|---|---|---|---|
| LSM Difference SEM [2] | Not Applicable | −1.806 | (1.297) | −0.926 | (0.769) | 0.284 | (0.980) | −0.348 | (0.784) | Not Calculated |

| | | | | | | |
|---|---|---|---|---|---|---|
| p-value [2] | Not Applicable | 0.1654 | 0.2297 | 0.7724 | 0.6575 | Not Calculated |

Percent Change from Baseline to Week 52 - EOT

| Mean (SD) | −5.13 | (41.749) | −7.66 | (36.536) | −12.11 | (33.682) | 11.78 | (42.188) | −4.38 | (37.677) | −4.43 (37.679) |
|---|---|---|---|---|---|---|---|---|---|---|---|
| Median | −11.85 | | −15.99 | | −19.00 | | 2.10 | | −11.72 | | −12.15 |
| Min, Max | −59.4, 131.2 | | −55.7, 54.0 | | −70.3, 100.0 | | −61.1, 148.9 | | −65.6, 104.9 | | −70.3, 148.9 |
| LSM (SEM) [2] | −8.18 | (5.43) | −19.48 | (11.06) | −11.20 | (4.96) | −1.32 | (7.73) | −5.46 | (5.20) | Not Calculated |

| | | | | | | | | | | |
|---|---|---|---|---|---|---|---|---|---|---|
| LSM Difference SEM [2] | Not Applicable | −11.300 | (11.924) | −3.014 | (7.068) | 6.858 | (9.012) | 2.718 | (7.211) | Not Calculated |

| | | | | | | |
|---|---|---|---|---|---|---|
| p-value [2] | Not Applicable | 0.3446 | 0.6703 | 0.4477 | 0.7067 | Not Calculated |

Note:
Baseline is the last assessment prior to the first dose of study drug.
Abbreviations: ELF = enhanced liver fibrosis. EOT = end of treatment, NIS4 = non-invasive score-4, PIIINP = amino-terminal propeptide of type III procollagen, QD = daily, QOD = every other day, TIMP1 = tissue inhibitor of metalloproteinases 1, ANCOVA = analysis of covariance, LSM = Least Squares Mean, QD = daily, QOD = every other day
[2] Results are from an Analysis of Covariance Model (ANCOVA) with randomization stratification factors (including whether the subject was randomized before/after the interim analysis), treatment assignments, baseline value, and the interaction of baseline value with treatment as covariates. The interaction of baseline value with treatment is not retained in the model if the p-value for the interaction is greater than the 0.15-level of significance and the model reran before estimates are computed.

TABLE 23

| | | | | | |
|---|---|---|---|---|---|
| Parameter | Placebo QD | 1.0 mg QD | 2.5 mg QD | 5.0 mg QOD | 10.0 mg QOD |
| Visit | (N = 65) | (N = 17) | (N = 66) | (N = 37) | (N = 61) |

Thyrotropin (uIU/mL)
Change from Baseline to Week 52 - EOT

| n | 47 | | 14 | | 55 | | 28 | | 49 | |
|---|---|---|---|---|---|---|---|---|---|---|
| LSM (SEM) [2] | 0.066 | (0.126) | −0.068 | (0.229) | −0.368 | (0.112) | 0.267 | (0.176) | −0.214 | (0.119) |

| | | | | | | | | | |
|---|---|---|---|---|---|---|---|---|---|
| LSM Difference (SEM) [2] | NA | −0.134 | (0.253) | −0.434 | (0.162) | 0.201 | (0.208) | −0.280 | (0.166) |

| | | | | | |
|---|---|---|---|---|---|
| p-value (VK2809 - Placebo) [2] | NA | 0.5988 | 0.0081 | 0.3346 | 0.0943 |

Percent Change from Baseline to Week 52 - EOT

| | | | | | | | | | | |
|---|---|---|---|---|---|---|---|---|---|---|
| LSM (SEM) [2] | 10.08 | (7.03) | −3.26 | (12.91) | −10.97 | (6.33) | 23.92 | (9.48) | −0.73 | (6.69) |

| | | | | | | | | | |
|---|---|---|---|---|---|---|---|---|---|
| LSM Difference (SEM) [2] | NA | −13.339 | (14.242) | −21.055 | (9.096) | 13.839 | (11.431) | −10.818 | (9.328) |

| | | | | | | |
|---|---|---|---|---|---|---|
| Parameter | Placebo QD | 1.0 mg QD | 2.5 mg QD | 5.0 mg QOD | 10.0 mg QOD | All VK2809 |
| Visit | (N = 65) | (N = 17) | (N = 66) | (N = 37) | (N = 61) | (N = 181) |

| | | | | | |
|---|---|---|---|---|---|
| p-value (VK2809 - Placebo) [2] | NA | 0.3502 | 0.0217 | 0.2276 | 0.2476 |

Thyroxine, Free (ng/dL)
Change from Baseline to Week 52 - EOT

| n | 47 | | 14 | | 55 | | 28 | | 49 | |
|---|---|---|---|---|---|---|---|---|---|---|
| LSM (SEM) [2] | 0.066 | (0.126) | −0.068 | (0.229) | −0.368 | (0.112) | 0.267 | (0.176) | −0.214 | (0.119) |

| | | | | | | | | | |
|---|---|---|---|---|---|---|---|---|---|
| LSM Difference (SEM) [2] | NA | −0.134 | (0.253) | −0.434 | (0.162) | 0.201 | (0.208) | −0.280 | (0.166) |

| | | | | | |
|---|---|---|---|---|---|
| p-value (VK2809 - Placebo) [2] | NA | 0.5988 | 0.0081 | 0.3346 | 0.0943 |

Percent Change from Baseline to Week 52 - EOT

| | | | | | | | | | | |
|---|---|---|---|---|---|---|---|---|---|---|
| LSM (SEM) [2] | 10.08 | (7.03) | −3.26 | (12.91) | −10.97 | (6.33) | 23.92 | (9.48) | −0.73 | (6.69) |

| | | | | | | | | | |
|---|---|---|---|---|---|---|---|---|---|
| LSM Difference (SEM) [2] | NA | −13.339 | (14.242) | −21.055 | (9.096) | 13.839 | (11.431) | −10.818 | (9.328) |

| | | | | | |
|---|---|---|---|---|---|
| p-value (VK2809 - Placebo) [2] | NA | 0.3502 | 0.0217 | 0.2276 | 0.2476 |

Triiodothyronine (pg/mL)
Change from Baseline to Week 52 - EOT

| n | 47 | | 14 | | 55 | | 28 | | 48 | |
|---|---|---|---|---|---|---|---|---|---|---|
| LSM (SEM) [2] | −0.09 | (0.063) | −0.01 | (0.116) | −0.07 | (0.057) | −0.04 | (0.086) | −0.06 | (0.061) |

| | | | | | | | | | |
|---|---|---|---|---|---|---|---|---|---|
| LSM Difference (SEM) [2] | NA | 0.08 | (0.128) | 0.02 | (0.082) | 0.05 | (0.103) | 0.03 | (0.084) |

| | | | | | |
|---|---|---|---|---|---|
| p-value (VK2809 - Placebo) [2] | NA | 0.5145 | 0.8262 | 0.6324 | 0.7505 |

Percent Change from Baseline to Week 52 - EOT

| | | | | | | | | | | |
|---|---|---|---|---|---|---|---|---|---|---|
| LSM (SEM) [2] | −1.82 | (1.80) | 0.18 | (3.31) | −1.06 | (1.62) | −0.81 | (2.42) | −0.39 | (1.74) |

| | | | | | | | | | |
|---|---|---|---|---|---|---|---|---|---|
| LSM Difference (SEM) [2] | NA | 2.00 | (3.645) | 0.76 | (2.322) | 1.01 | (2.907) | 1.43 | (2.400) |

| | | | | | |
|---|---|---|---|---|---|
| p-value (VK2809 - Placebo) [2] | NA | 0.5841 | 0.7446 | 0.7290 | 0.5523 |

Sex Hormone Binding Globulin (nmol/L)
Change from Baseline to Week 52 - EOT

| n | 47 | | 14 | | 53 | | 27 | | 48 | |
|---|---|---|---|---|---|---|---|---|---|---|
| LSM (SEM) [2] | −0.286 | (6.673) | 45.174 | (14.285) | 71.180 | (6.382) | 49.401 | (9.270) | 88.338 | (6.392) |

| | | | | | | | | | |
|---|---|---|---|---|---|---|---|---|---|
| LSM Difference (SEM) [2] | NA | 45.461 | (15.302) | 71.466 | (8.800) | 49.688 | (10.960) | 88.624 | (8.887) |

| | | | | | |
|---|---|---|---|---|---|
| p-value (VK2809 - Placebo) [2] | NA | 0.0034 | <0.0001 | <0.0001 | <0.0001 |

Percent Change from Baseline to Week 52 - EOT

| | | | | | | | | | | |
|---|---|---|---|---|---|---|---|---|---|---|
| LSM (SEM) [2] | −4.36 | (24.43) | 97.80 | (45.23) | 185.91 | (22.48) | 127.27 | (33.48) | 302.82 | (23.41) |

| | | | | | | | | | |
|---|---|---|---|---|---|---|---|---|---|
| LSM Difference (SEM) [2] | NA | 102.163 | (49.686) | 190.266 | (31.828) | 131.631 | (39.889) | 307.176 | (32.563) |

| | | | | | |
|---|---|---|---|---|---|
| p-value (VK2809 - Placebo) [2] | NA | 0.0412 | <0.0001 | 0.0012 | <0.0001 |

Abbreviations: ANCOVA = analysis of covariance, LSM = Least Squares Mean, QD = daily, QOD = every other day
[2] Results are from an Analysis of Covariance Model (ANCOVA) with randomization stratification factors (including whether the subject was randomized before/after the interim analysis), treatment assignments, baseline value, and the interaction of baseline value with treatment as covariates. The interaction of baseline value with treatment is not retained in the model if the p-value for the interaction is greater than the 0.15-level of significance and the model reran before estimates are computed.

In the VK2809 arms, both primary and secondary endpoints showed reductions in relative and absolute fat content at Weeks 12 and 52 as assessed by MRI-PDFF.

At Week 12, statistically significant reductions in relative and absolute liver fat content in the VK2809 arms versus placebo were noted as assessed by MRI-PDFF in the Full Analysis Set. The mean relative reduction ranged from 19.3% to 53.0% in the VK2809 arms. Reductions increased with increasing dose. At Week 12, a sensitivity analysis to determine tipping point was consistent with the reduction in the relative change in liver fat content from baseline. A tipping point of 81% and higher was determined in which no further statistically significant reductions in VK2809 versus placebo were observed. At Week 12, the number of participants with a ≥30% fat reduction response rate for VK2809 arms was 75.3% versus 13.6% in the placebo arm (p<0.0001).

At Week 52, statistically significant reductions in relative and absolute liver fat content in the VK2809 arms versus placebo were noted as assessed by MRI-PDFF in the Full Analysis Set. The mean relative reduction ranged from 30.1% to 53.0% in the VK2809 arms. Reductions increased with increasing dose. At Week 52, the number of participants with a ≥30% fat reduction response rate for VK2809 arms was 79.3% versus 27.1% in the placebo arm (p<0.0001).

For the secondary endpoints that were assessed by liver biopsy at Week 52, the following results were noted. Steatohepatitis resolution with no worsening of fibrosis response rate was 68.6% in VK2809 arms versus 29.3% in the placebo arm (p<0.0001). ≥1-stage of fibrosis improvement and no worsening steatohepatitis response rate was 51.1% in the VK2809 arms versus 34.1% in the placebo arm (p=0.0308). ≥2 point NAS improvement with no worsening fibrosis response rate was 75.2% in the VK2809 arms versus 34.1% in the placebo arm (p<0.0001). Resolution of ≥1-stage steatohepatitis and fibrosis improvement response rate was 43.8% in the VK2809 arms versus 19.5% in the placebo arm (p=0.0032).

Sensitivity analyses were conducted after the study was unblinded were consistent with liver biopsy results. Steatohepatitis and fibrosis showed improvements at Week 52. The following results were noted. Resolution of steatohepatitis with no worsening fibrosis response rate was 56.0% in the VK2809 arms versus 21.1% in the placebo arm (p<0.0001). ≥1-stage of fibrosis improvement and no worsening steatohepatitis response rate was 41.7% in the VK2809 arms versus 24.6% in the placebo arm (p=0.0101). ≥2-point NAS improvement with no worsening fibrosis response rate was 61.3% in the VK2809 arms versus 24.6% in the placebo arm (p<0.0001). Resolution of ≥1-stage steatohepatitis and fibrosis improvement (response rate was 35.7% in the VK2809 arms versus 14.0% in the placebo arm (p=0.0009). Ad hoc analyses conducted after the data were unblinded were consistent with the prior analyses for fibrosis and steatohepatitis.

For the exploratory endpoints that were assessed, the following results were noted. For MRI-PDFF, the normalization of liver fat response rate was 12.7% (CI: 8.43, 18.57) at Week 12 and 17.9% (CI: 12.54, 24.98) at Week 52 in the VK2809 arms. The response rate was 0 at Week 12 and 2.1% (CI: 0.37, 10.90) at Week 52 in the placebo arm. For MRI-PDFF, the absolution reduction of liver fat ≥5% response rate was 73.5% at Week 12 and 76.6% at Week 52 in the VK2809 arms. The response rate was versus 15.3% at Week 12 and 27.1% in Week 52 in the placebo arm. For liver biopsy at Week 52, the response rate for ≥2 point or improvement in NAS was 84.2% in the VK2809 arms versus 33.3% in the placebo arm. With the addition of a 1-stage improvement in liver fibrosis, the response rate was 48.2% in the VK2809 treatment arms versus 24.4% in the placebo arm. A sensitivity analysis of the ≥2-point improvement showed a response rate of 69.6% in the VK2809 arms versus 24.6% in the placebo arm. With the addition of a 1-stage improvement in liver fibrosis, the response rate at Week 52 was 39.3% in the VK2809 treatment arms versus 17.5% in the placebo arm.

For paired liver biopsy comparison, the following results and improvements were noted:
NASH/MASH Improvements: VK2809 treatment arms 55.8% vs. placebo arm 30.0%;
Steatosis Improvements: VK2809 treatment arms 57.9% vs. placebo arm 46.7%;
Hepatocyte ballooning Improvements: VK2809 treatment arms 56.9% vs. placebo arm 34.0%;
Inflammation Improvement: VK2809 treatment arms 44.2% vs. placebo arm 22.0%;
Fibrosis Improvement: VK2809 treatment arms 36.5% vs. placebo arm 32.0%.

For NASH biomarkers from baseline to Week 52, the following results were noted. ELF scores (mean, median, LSM [absolute change], LSM difference [relative change]) were reduced at Week 52 but greater reduction was noted for participants in the VK2809 treatment arms versus the placebo arm. Pro C3 reductions in all treatment arms were noted for absolute and relative mean, median, LSM, and LSM difference. PINP reductions in all treatment arms including placebo, except for the VK2809 5 mg QOD arm. TIMP1/creatinine reductions in all treatment arms including placebo, except the VK2809 1 mg QD arm. NIS4 reductions were noted for absolute mean, median, LSM, and LSM mean differences for all treatment arms.

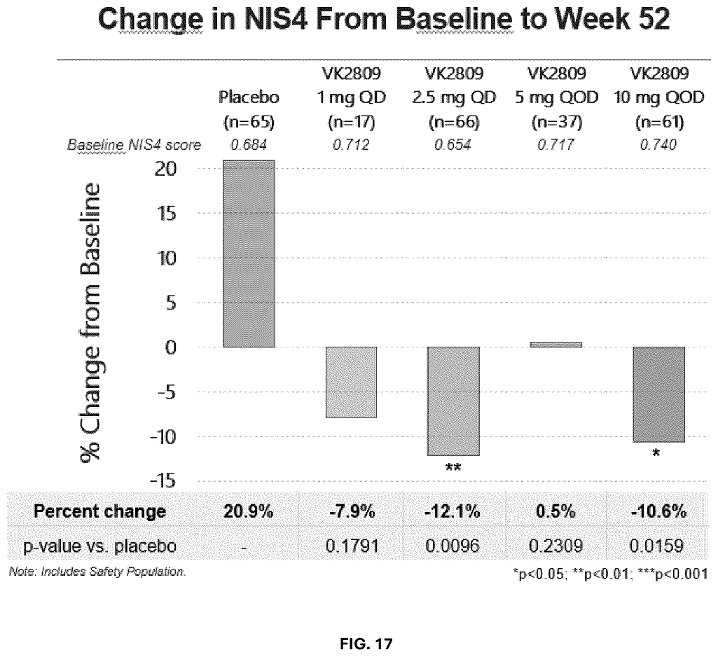

FIG. 17 shows the change in NIS4 from baseline to week 52. As shown, there was a statistically significant reduction in NIS4 for the 2.5 mg QD dose and the 10 mg QOD dose. The percentage change from baseline for the 1 mg QD dose was 7.9% reduction, for the 2.5 mg QD dose was 12.1% reduction, for the 5 mg QOD dose was 0.5% increase, and for the 10 mg QOD dose was 10.6% reduction.

For the liver profile, the following results were noted. Absolute and relative reductions in ALT and AST were noted in all treatment arms but changes were greater with VK2809 treatment. Absolute and relative reductions in GGT were noted in the VK2809 arms but not the placebo arm. Reductions from baseline in albumin, prothrombin time, prothrombin international normalized ratio were noted in all treatment arms. Increases from baseline were noted for direct bilirubin with greater changes in the VK2809 arms.

For thyroid profile, the following results were noted. Absolute and relative LSM reductions in T3 were observed in all treatment arms except the 1 mg VK2809 arm. Absolute and relative LSM increases in SHBG were observed in all treatment arms except the 1 mg VK2809 arm.

For lipid profile, the following results were noted. Overall, lipid measurements of mean and median HDL and LDL cholesterol, total cholesterol, triglycerides, lipoprotein A, and apolipoprotein B improved in all treatment arms. However, the changes were more pronounced in the VK2809 treatment arms with greater differences noted at the higher VK2809 dose. Absolute and relative increases in HDL cholesterol were noted in all treatment arms. LSM differences were notable for the 10 mg VK2809 treatment arm (p<0.103). Absolute and relative decreases in LDL cholesterol were noted in all treatment arms except relative change from baseline in the placebo arm. LSM differences were also notable in all VK2809 treatment arms (p≤0.034). Absolute and relative decreases in cholesterol were noted in all treatment arms. LSM differences were also notable in all VK2809 treatment arms (p≤0.0205). Absolute and relative decreases in triglycerides were noted in all VK2809 treatment arms. LSM differences were also notable VK2809 2.5 mg QD and 10.0 mg QOD arms (p≤0.0056). Absolute and relative LSM decreases in lipoprotein-a were noted in all VK2809 treatment arms but not placebo arms. LSM differences were also notable in all VK2809 treatment arms except the 1.0 mg QD arm (p≤0.0011). Absolute and relative LSM decreases in apolipoprotein B were noted in all treatment arms. LSM differences were also notable in all VK2809 treatment arms (p≤0.0074).

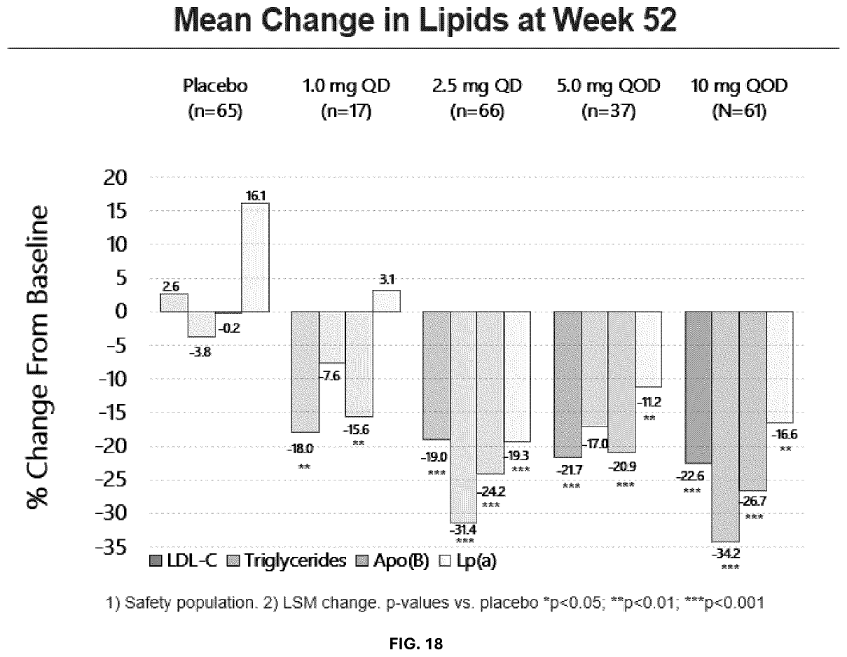

FIG. 18 shows a mean change in lipids at week 52. The results show a robust reduction in plasma lipids where the plasma lipids significantly improved after 52 weeks. The percent change from baseline of LDL-C was statistically significant for the 1.0 mg QD dose, the 2.5 mg QD dose, the 5.0 mg QOD dose, and the 10 mg QOD dose. The percent change from baseline of triglycerides was statistically significant for the 2.5 mg QD dose and the 10 mg QOD dose. The percent change form baseline of apo(β) was statistically significant for the 1.0 mg QD dose, 2.5 mg QD dose, the 5.0 mg QOD dose, and the 10 mg QOD dose. The percent change from baseline of 1p(α) was statistically significant for the 2.5 mg QD dose, the 5.0 mg QOD dose, and the 10 mg QOD dose. The reduction in plasma lipids at 52 weeks suggests long-term cardioprotective benefit.

All participants were required to provide predose samples for plasma concentration analysis (Sparse PK collection) at designated time points. Participants were given the option to perform intensive PK sampling on Day 1 and Week 52, targeting approximately 6 participants per active treatment arm. Most participants provided predose samples for most visits, although samples were not collected for reasons such as remote visits or per protocol amendments.

In the VK2809 arms, the Sparse PK Population comprised 159 participants and the Intensive PK Population comprised the following 14 participants:
VK2809 1.0 mg QD: n=1 participant;
VK2809 2.5 mg QD: n=5 participants;
VK2809 5.0 mg QOD: n=3 participants;
VK2809 10.0 mg QOD: n=5 participants.

All 14 participants in the Intensive PK Population provided some samples for Day 1. At Week 52, samples were collected from the 2.5 mg QD (n=3) and 10.0 mg QOD treatment (n=4) VK2809 arms. Interpretation of collective results is limited with small sample sizes.

Evidence of systemic exposure to VK2809 and VK2809A was observed in all treated participants. Geometric means of predose concentrations were several-fold greater for the active metabolite, VK2809A, than for the prodrug, VK2809. In general, VK2809 appears to be rapidly absorbed and converted to VK2809A.

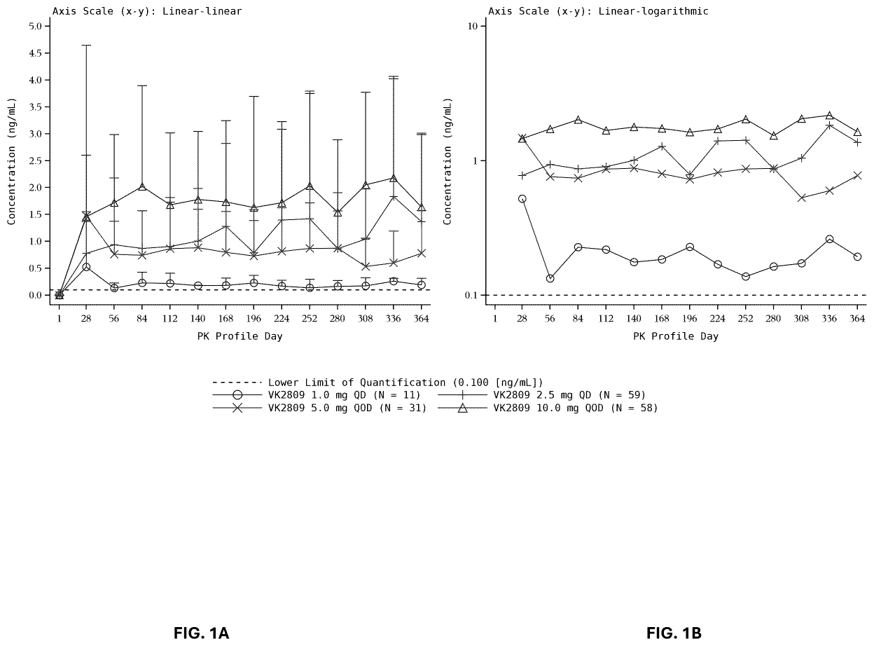

A summary of predose VK2809 plasma concentration data with geometric means and CVs are presented in Table 24. Predose linear and log-linear plots of the mean (+SD) overlaying dose as a function of days from Day 1 of Week 1 through Day 364 of Week 52 are presented in FIGS. 1A and 1B, respectively.

Observed steady state appeared to be achieved by Day 28. The 2.5 mg QD and 5.0 mg QOD arms demonstrated observable comparable profiles although the 5.0 mg QOD arm was more variable. The 10.0 mg QOD arm profile appeared to be dose proportional versus the 2.5 mg QD and 5.0 mg QD arms.

TABLE 24

| Timepoint N | | | | | | | |
|---|---|---|---|---|---|---|---|
| Geometric Mean | 1.0 mg QD | | 2.5 mg QD | | 5.0 mg QOD | 10.0 mg QD | |
| (Geometric CV %) | (N = 11) | | (N = 59) | | (N = 31) | (N = 58) | |
| Day 1 n | 8 | | 37 | | 16 | 43 | |
| Mean (CV %) | NC | (NC) | NC | (NC) | NC (NC) | NC | (NC) |
| Week 4 n | 5 | | 39 | | 16 | 36 | |
| Mean (CV %) | 0.322 | (214.5) | 0.65 | (78.6) | 0.815 (137.9) | 1.27 | (70.2) |
| Week 8 n | 8 | | 41 | | 23 | 43 | |
| Mean (CV %) | 0.169 | (35.2) | 0.76 | (72.5) | 0.618 (102.1) | 1.36 | (88.4) |
| Week 12 n | 9 | | 42 | | 23 | 42 | |
| Mean (CV %) | 0.257 | (55.5) | 0.728 | (67.9) | 0.603 (121.1) | 1.74 | (79.5) |
| Week 16 n | 10 | | 44 | | 25 | 45 | |
| Mean (CV %) | 0.237 | (57.4) | 0.732 | (77.7) | 0.682 (111.8) | 1.32 | (97.0) |
| Week 20 n | 7 | | 16 | | 20 | 21 | |
| Mean (CV %) | 0.176 | (6.2) | 0.78 | (96.2) | 0.583 (132.1) | 1.55 | (72.1) |
| Day 24 n | 7 | | 12 | | 17 | 21 | |
| Mean (CV %) | 0.196 | (45.7) | 0.899 | (120.4) | 0.528 (123.7) | 1.48 | (114.7) |
| Week 28 n | 9 | | 47 | | 25 | 47 | |
| Mean (CV %) | 0.205 | (46.4) | 0.699 | (66.6) | 0.583 (115.1) | 1.34 | (94.0) |
| Week 32 n | 5 | | 16 | | 17 | 13 | |
| Mean (CV %) | 0.205 | (32.3) | 0.901 | (107.3) | 0.527 (126.8) | 1.36 | (105.6) |
| Week 36 n | 4 | | 17 | | 13 | 12 | |
| Mean (CV %) | 0.274 | (11.4) | 0.866 | (100.3) | 0.589 (112.9) | 1.66 | (100.5) |
| Week 40 n | 10 | | 45 | | 25 | 44 | |
| Mean (CV %) | 0.193 | (38.1) | 0.766 | (65.5) | 0.641 (142.8) | 1.36 | (94.9) |
| Week 44 n | 3 | | 13 | | 13 | 7 | |
| Mean (CV %) | 0.258 | (2.5) | 00.754 | (104.9) | 0.452 (106.2) | 1.3 | (169.5) |
| Week 48 n | 3 | | 9 | | 8 | 9 | |
| Mean (CV %) | 0.257 | (21.4) | 1.1 | (135.3) | 0.495 (107.4) | 1.52 | (203.2) |
| Week 52 n | 9 | | 35 | | 16 | 34 | |
| Mean (CV %) | 0.197 | (51.8) | 0.941 | (99.0) | 0.556 (115.5) | 1.51 | (92.6) |

Notes:
This table includes subjects with predose concentration data included in descriptive statistics. Concentrations below the lower limit of quantitation are treated as 0 for the purpose of summary statistics.
Abbreviations: CV = covariance, n = number of observations, NC = not calculated, QD = daily, QOD = every other day

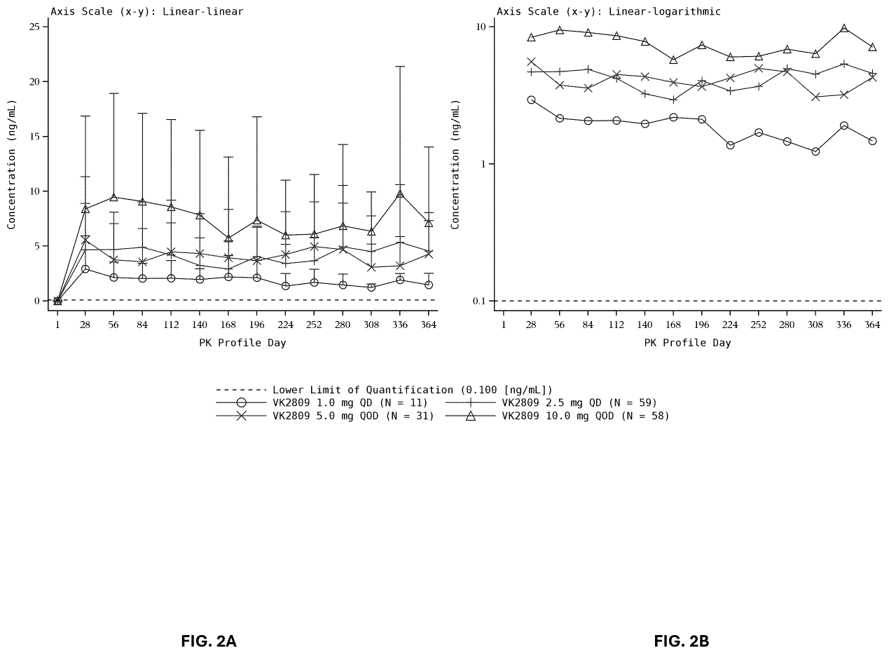

A summary of predose VK2809A plasma concentration data using geometric means and CVs are summarized in Table 25. Predose linear and log-linear plots of the mean (+SD) overlaying dose as a function of days are presented in FIGS. 2A and 2B, respectively.

Observed steady state appeared to be achieved by Day 28. The 5.0 mg QOD and 2.5 mg QD arms demonstrated observable comparable profiles. The 10.0 mg QOD arm profile appeared to be slightly less than dose proportional versus the 2.5 mg QD and 5.0 QOD arms.

TABLE 25

| Timepoint N | | | | | | | |
|---|---|---|---|---|---|---|---|
| Geometric Mean | 1.0 mg QD | | 2.5 mg QD | | 5.0 mg QOD | | 10.0 mg QD |
| (Geometric CV %) | (N = 11) | | (N = 59) | | (N = 31) | | (N = 58) |
| Day 1 n | 7 | | 37 | | 16 | | 43 |
| Mean (CV %) | NC | (NC) | NC | (NC) | NC | (NC) | NC (NC) |
| Week 4 n | 5 | | 39 | | 16 | | 36 |
| Mean (CV %) | 1.63 | (213.1) | 3.74 | (91.4) | 4.78 | (85.2) | 5.58 (140.9) |
| Week 8 n | 8 | | 41 | | 23 | | 43 |
| Mean (CV %) | 1.76 | (79.2) | 3.79 | (84.2) | 2.87 | (100.9) | 6.08 (133.1) |
| Week 12 n | 9 | | 42 | | 23 | | 42 |
| Mean (CV %) | 1.97 | (75.2) | 3.79 | (87.4) | 2.71 | (122.0) | 6.56 (124.4) |
| Week 16 n | 10 | | 44 | | 25 | | 45 |
| Mean (CV %) | 1.83 | (87.9) | 3.64 | (69.8) | 3.15 | (146.9) | 6.05 (121.0) |
| Week 20 n | 7 | | 16 | | 20 | | 21 |
| Mean (CV %) | 1.68 | (75.7) | 2.63 | (101.1) | 2.83 | (133.9) | 5.48 (114.6) |
| Week 24 n | 7 | | 12 | | 17 | | 21 |
| Mean (CV %) | 2.03 | (86.8) | 2.35 | (107.7) | 2.55 | (115.5) | 3.82 (167.9) |
| Week 28 n | 9 | | 47 | | 25 | | 47 |
| Mean (CV %) | 1.67 | (86.2) | 3.66 | (71.4) | 2.61 | (123.8) | 5.13 (127.3) |
| Week 32 n | 5 | | 16 | | 17 | | 13 |
| Mean (CV %) | 1.51 | (62.7) | 3.01 | (56.9) | 2.99 | (103.3) | 4.56 (125.5) |
| Week 36 n | 4 | | 17 | | 13 | | 12 |
| Mean (CV %) | 2.22 | (23.4) | 3.22 | (55.2) | 3.64 | (99.2) | 4.84 (105.8) |
| Week 40 n | 10 | | 45 | | 25 | | 44 |
| Mean (CV %) | 1.37 | (72.7) | 3.80 | (98.1) | 2.80 | (152.4) | 4.61 (160.2) |
| Week 44 n | 3 | | 13 | | 13 | | 7 |
| Mean (CV %) | 1.20 | (28.1) | 3.29 | (117.0) | 2.22 | (127.26) | 4.84 (124.0) |
| Week 48 n | 3 | | 9 | | 8 | | 9 |
| Mean (CV %) | 1.84 | (30.8) | 3.85 | (100.7) | 2.46 | (88.9) | 6.37 (196.7) |
| Week 52 n | 9 | | 35 | | 16 | | 34 |
| Mean (CV %) | 1.41 | (66.4) | 3.68 | (82.7) | 3.33 | (86.6) | 5.49 (117.2) |

Notes:
This table includes subjects with predose concentration data included in descriptive statistics. Concentrations below the lower limit of quantitation are treated as 0 for the purpose of summary statistics .
Abbreviations: CV = covariance, n = number of observations, NC = not calculated, QD = daily, QOD = every other day

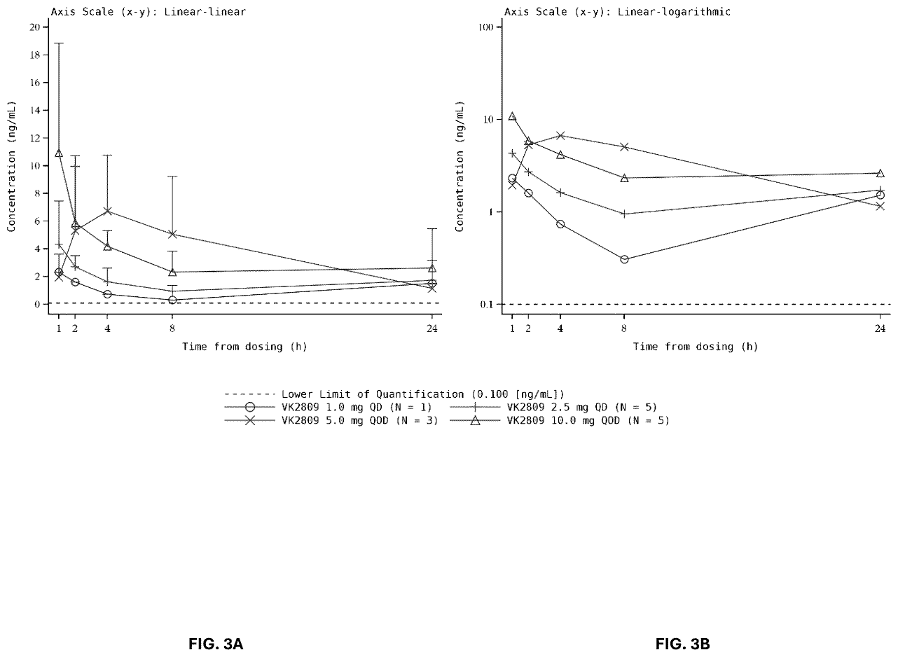

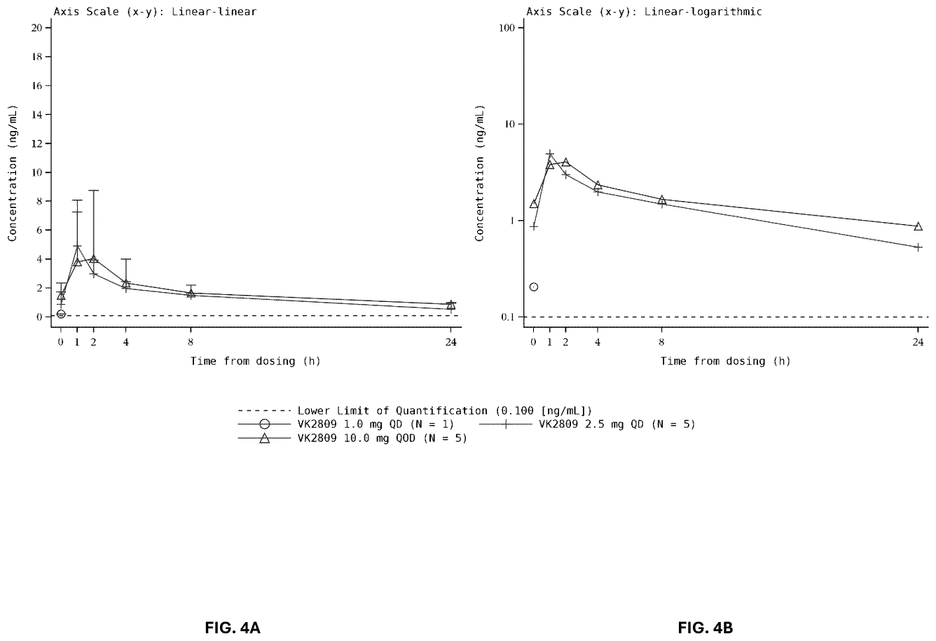

FIGS. 3A, 3B, 4A, and 4B show data from Day 1 of Week 1 and data from Day 364 of Week 52, in the form of linear and log-linear time profile plots of the mean (+SD) overlaying dose as a function of time (in hours) from dosing. FIG. 3A shows linear plot of Day 1 (Week 1) data and FIG. 3B shows log-linear plot of Day 1 (Week 1) data. FIG. 4A shows linear plot of Day 364 (Week 52) data and FIG. 4B shows log-linear plot of Day 52 (Week 52) data. On Day 1, 2 participants in each of the 2.5 mg QD and 10.0 mg QOD arms showed 24 hour plasma concentrations higher than the 8 hour values. Except for the 5.0 mg QOD arm, the concentration profiles exhibited similar properties in a less than dose proportional manner. The 5.0 mg QOD arm had an apparent tmax that was greater than the other arms. At Day 364 of Week 52, profiles of the 2.5 mg QD and 10.0 mg QOD arms were very similar and appear less than dose proportional. Upon observation of the 10.0 mg QOD arm, 3 participants had Day 364 (at Week 52) profiles much lower than the Day 1 (at Week 1) mean profile.

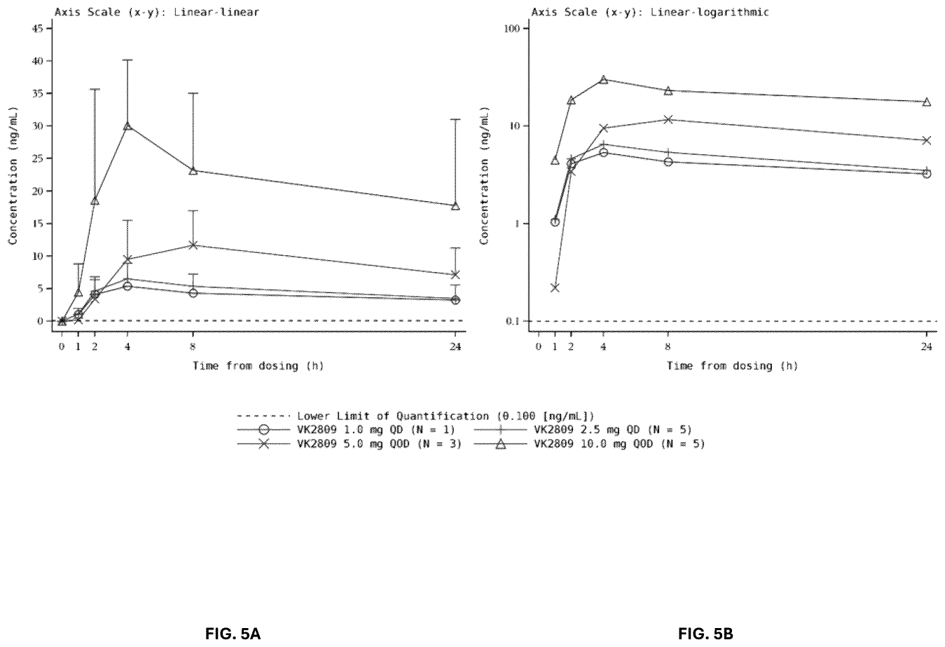

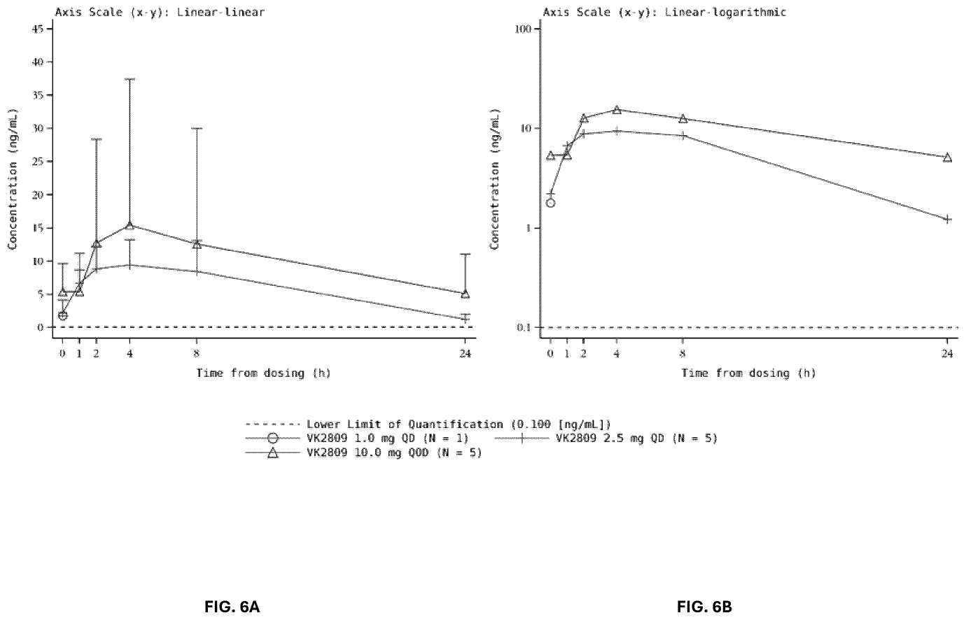

Linear and log-linear time profile plots of the mean (+SD) overlaying dose are presented for Day 1 (Week 1) in FIGS. 5A and 5B, respectively, and for Day 364 (Week 52) in FIGS. 6A and 6B, respectively. Day 364 (Week 52) plasma concentration data were not available in the 1.0 mg QD and 5.0 mg QOD arms since all participants discontinued early. Linear and log-linear individual time profile figures including the Day 1 and Week 52 and all treatment arms are presented in.

At Day 1 of Week 1, profiles of the 1.0 mg QD, 2.5 mg QD, 5.0 mg QOD, and 10.0 mg QOD arms showed an increase of exposure with an increase in dose, with the 10.0 mg dose greater than dose proportional.

At Day 364 of Week 52, profiles of the 2.5 mg QD and 10.0 mg QOD treatment arms appear less than dose proportional. Upon observation of the 10.0 mg QOD treatment arm, 3 participants demonstrated profiles from Day 364 at Week 52 had much lower values than the Day 1 mean profile. Plasma concentration data from Day 364 at Week 52 were not available in the 1.0 mg QD and 5.0 mg QOD arms since all participants in discontinued early. Data for AUC0-48 was reported only for the 10.0 mg QOD arm since no other arms had sampling up to 48 hours at Week 52.

Summaries of PK parameters are presented on the Day 1 in Table 26 and Day 364 at Week 52 in Table 27. On Day 1, AUClast, AUC0-24h, and Cmax increased in an approximate dose proportional manner with respect to the 2.5 mg QD and 5.0 mg QOD arms. The 10.0 mg QOD arm has slightly lower AUCs than the 5.0 mg QOD arm. Higher variability was observed in the higher dose arms. The median tmax ranged from 1 to 2 hours with 1 participant in the 10.0 mg QOD arm having a value at approximately 24 hours.

On Day 364 at Week 52, the AUClast, and AUC0-24 h values were similar with respect to the 2.5 mg QD and 10.0 mg QOD arms. The 10.0 mg QOD arm had a lower Cmax relative to the 2.5 mg QD arm. The tmax ranged from 1 to 2 hours. The accumulation ratio base within the 2.5 mg QD arm showed little to no accumulation (median=1.04). The 10.0 mg QOD treatment arm appeared to have lower exposures at Week 52 compared to Day 1 (median=0.633).

A summary of PK parameters are presented on Day 1 in Table 28 and Day 364 at Week 52 in Table 29. On Day 1, AUClast, and AUC0-24 h increased in an approximate dose proportional manner with Cmax increasing greater than dose proportional with respect to the 10.0 mg QOD arm. AUC0-48 was reported only for the 10.0 mg QOD arm since no other treatments had sampling up to 48 hours a day 364 at Week 52. The median tmax ranged from 4 to 8 hours. One participant in the 10.0 mg QOD arm had a tmax value at approximately 24 hours.

On Day 1, the median AUClast metabolite to parent ratios ranged from 2.46 to 6.64 with the highest ratio noted in the 10.0 mg QOD arm and similar ratios for the 2.5 mg QD and 5.0 mg QOD arms. Median Cmax metabolite to parent ratios ranged from 1.5 to 2.65 with the highest ratio noted in the 10.0 mg QOD arm and similar ratios for the 2.5 mg QD and 5.0 mg QOD arms.

At Day 364 at Week 52, AUClast, AUC0-24 h and Cmax were comparable with respect to the 2.5 mg QD and 10.0 mg QOD arms. The median tmax ranged from 0.49 to 4 hours. Median AUClast metabolite to parent ratios were very similar (3.63 and 3.55 for the 2.5 mg QD and 10.0 mg QOD arms, respectively). Median Cmax metabolite to parent ratios ranged from 1.96 to 3.12 with the higher ratio noted in the 10.0 mg QOD arm.

At Day 364 at Week 52, within the 2.5 mg QD arm, some accumulation was noted (median value=1.39). The 10.0 mg QOD arm appeared to have lower exposures at Week 52 versus Day 1 (median value=0.218).

TABLE 26

| | | | | |
|---|---|---|---|---|
| Parameter (unit) | 1.0 mg QD | 2.5 mg QD | 5.0 mg QOD | 10.0 mg QOD |
| Statistics | (N = 1) | (N = 5) | (N = 3) | (N = 5) |

AUCₗₐₛₜ

| | | | | |
|---|---|---|---|---|
| n | 1 | 4 | 3 | 5 |
| Geometric Mean | NC | 35.4 | 75.6 | 66.9 |
| Geometric CV | NC | 51.3 | 48.0 | 57.7 |
| Arithmetic Mean (SD) | NC (NC) | 38.8 (19.7) | 81.3 (39.6) | 75.1 (40.6) |
| Median | NC | 32.9 | 63.9 | 74.4 |
| Min, Max | 22.1, 22.1 | 23.6, 65.7 | 53.4, 127 | 35.0, 139 |

AUC_{0-24 h}

| | | | | |
|---|---|---|---|---|
| n | 1 | 4 | 3 | 5 |
| Geometric Mean | NC | 35.4 | 75.5 | 67.1 |
| Geometric CV | NC | 51.3 | 48.2 | 57.5 |
| Arithmetic Mean (SD) | NC (NC) | 38.8 (19.7) | 81.3 (39.6) | 75.2 (40.5) |
| Median | NC | 32.9 | 63.9 | 74.9 |
| Min, Max | 21.8, 21.8 | 23.6, 65.7 | 53.2, 127 | 35.2, 139 |

tₘₐₓ

| | | | | |
|---|---|---|---|---|
| n | 1 | 5 | 3 | 5 |
| Geometric Mean | NC | NC | NC | NC |
| Geometric CV | NC | NC | NC | NC |
| Arithmetic Mean (SD) | NC (NC) | 1.40 (0.55) | 2.72 (1.11) | 5.71 (9.98) |
| Median | NC | 1.00 | 2.17 | 1.00 |
| Min, Max | 0.82, 0.82 | 1.00, 2.00 | 2.00, 4.00 | 1.00, 23.55 |

Cₘₐₓ

| | | | | |
|---|---|---|---|---|
| n | 1 | 5 | 3 | 5 |
| Geometric Mean | NC | 4.06 | 8.11 | 10.2 |
| Geometric CV | NC | 70.6 | 53.9 | 76.2 |
| Arithmetic Mean (SD) | NC (NC) | 4.72 (2.63) | 8.75 (3.68) | 12.0 (6.63) |
| Median | NC | 5.29 | 10.3 | 13.3 |
| Min, Max | 2.32, 2.32 | 1.99, 8.11 | 4.54, 11.4 | 3.98, 19.7 |

Abbreviations: AR_{AUC} = accumulation ratio base on AUC₀₋₂₄, CV = Coefficient of Variation, EOT = End of Treatment, Max = Maximum, Min = Minimum, N = Number of subjects in the respective group, n = Number of Observations, NC = Not Calculated, QD = Every Day, QOD = Every Other Day, SD = Standard Deviation. AUCₗₐₛₜ = area under the concentration-time curve from hour 0 to the last measurable concentration, AUC_{0-24 h} or AUC_{0-48 h} = area under the concentration-time curve from 0 to 24 or 48 hours postdose(QOD cohorts, Week 52 only), Cₘₐₓ = maximum observed plasma concentration, Tₘₐₓ = time to maximum observed plasma concentration, Tₗₐₛₜ = time of last measurable plasma concentration

TABLE 27

| | | | | |
|---|---|---|---|---|
| Parameter (unit) | 1.0 mg QD | 2.5 mg QD | 5.0 mg QOD | 10.0 mg QOD |
| Statistics | (N = 1) | (N = 5) | (N = 3) | (N = 5) |

AUCₗₐₛₜ

| n | 0 | 4 | | 0 | 4 |
|---|---|---|---|---|---|
| Geometric Mean | NA | 34.3 | | NA | 37.5 |
| Geometric CV | NA | 25.9 | | NA | 41.2 |
| Arithmetic Mean (SD) | NA | 35.0 | (8.23) | NA | 40.0 (18.5) |

| | | | | |
|---|---|---|---|---|
| Median | NA | 38.7 | NA | 31.0 |
| Min, Max | NA | 25.6, 40.8 | NA | 30.3, 67.8 |

AUC_{0-24 h}

| n | 0 | 4 | | 0 | 4 |
|---|---|---|---|---|---|
| Geometric Mean | NA | 34.4 | | NA | 37.5 |
| Geometric CV | NA | 26.5 | | NA | 41.2 |
| Arithmetic Mean (SD) | NA | 35.2 | (8.41) | NA | 40.0 (18.5) |

| | | | | |
|---|---|---|---|---|
| Median | NA | 39.3 | NA | 31.0 |
| Min, Max | NA | 25.2, 40.8 | NA | 30.3, 67.8 |

AUC_{0-48 h}

| | | | | |
|---|---|---|---|---|
| n | 0 | 0 | 0 | 4 |
| Geometric Mean | NA | NA | NA | 53.4 |
| Geometric CV | NA | NA | NA | 23.2 |
| Arithmetic Mean (SD) | NA | NA | NA | 54.5 (13.7) |
| Median | NA | NA | NA | 48.8 |
| Min, Max | NA | NA | NA | 45.9, 74.7 |

tmax

| | | | | |
|---|---|---|---|---|
| n | 0 | 3 | 0 | 4 |
| Geometric Mean | NA | NC | NA | NC |
| Geometric CV | NA | NC | NA | NC |
| Arithmetic Mean (SD) | NA | NA | NA | 1.00 (1.15) |
| Median | NA | NA | NA | 1.00 |
| Min, Max | NA | NA | NA | 0.00, 2.00 |

Cmax

| n | 0 | 3 | | 0 | 4 |
|---|---|---|---|---|---|
| Geometric Mean | NA | 4.56 | | NA | 2.93 |
| Geometric CV | NA | 51.3 | | NA | 110.5 |
| Arithmetic Mean (SD) | NA | 4.92 | (2.33) | NA | 4.19 (4.61) |

| | | | | |
|---|---|---|---|---|
| Median | NA | 4.50 | NA | 1.95 |
| Min, Max | NA | 2.83, 7.44 | NA | 1.76, 11.1 |

AR_{AUC}

| n | 0 | 3 | | 0 | 4 |
|---|---|---|---|---|---|
| Arithmetic Mean (SD) | NA | 1.24 | (0.425) | NA | 0.741 (0.571) |

| | | | | |
|---|---|---|---|---|
| Median | NA | 1.04 | NA | 0.633 |
| Min, Max | NA | 0.957, 1.73 | NA | 0.218, 1.48 |

Abbreviations: AR_{AUC} = accumulation ratio base on AUC₀₋₂₄, CV = Coefficient of Variation, EOT = End of Treatment, Max = Maximum, Min = Minimum, N = Number of subjects in the respective group, n = Number of Observations, NC = Not Calculated, QD = Every Day, QOD = Every Other Day, SD = Standard Deviation. AUCₗₐₛₜ = area under the concentration-time curve from hour 0 to the last measurable concentration, AUC_{0-24 h} or AUC_{0-48 h} = area under the concentration-time curve from 0 to 24 or 48 hours postdose(QOD cohorts, Week 52 only), Cₘₐₓ = maximum observed plasma concentration, Tₘₐₓ = time to maximum observed plasma concentration, Tₗₐₛₜ = time of last measurable plasma concentration

TABLE 28

| | | | | |
|---|---|---|---|---|
| Parameter (unit) | 1.0 mg QD | 2.5 mg QD | 5.0 mg QOD | 10.0 mg QOD |
| Statistics | (N = 1) | (N = 5) | (N = 3) | (N = 5) |

AUCₗₐₛₜ and AUC_{0-24 h}

| n | 1 | 4 | | 3 | | 5 |
|---|---|---|---|---|---|---|
| Geometric Mean | NC | 102 | | 186 | | 444 |
| Geometric CV | NC | 52.7 | | 58.2 | | 39.3 |
| Arithmetic Mean (SD) | NC (NC) | 110 | (41.4) | 204 | (105) | 467 (145) |

| Median | NC | 127 | 191 | 496 |
|---|---|---|---|---|
| Min, Max | 92.9, 92.9 | 48.7, 137 | 107, 315 | 233, 626 |

tₘₐₓ

| n | 1 | 5 | | 3 | | 5 |
|---|---|---|---|---|---|---|
| Geometric Mean | NC | NC | | NC | | NC |
| Geometric CV | NC | NC | | NC | | NC |
| Arithmetic Mean (SD) | NC (NC) | 4.69 | (1.57) | 8.06 | (0.10) | 8.65 (8.49) |

| | | | | |
|---|---|---|---|---|
| Median | NC | 4.00 | 8.00 | 4.00 |
| Min, Max | 3.82, 3.82 | 3.95, 7.50 | 8.00, 8.17 | 3.97, 23.55 |

Cₘₐₓ

| n | 1 | 5 | | 3 | | 5 |
|---|---|---|---|---|---|---|
| Geometric Mean | NC | 6.16 | | 10.8 | | 30.1 |
| Geometric CV | NC | 42.4 | | 51.9 | | 35.5 |
| Arithmetic Mean (SD) | NC (NC) | 6.54 | (2.31) | 11.7 | (5.32) | 31.5 (9.66) |

| | | | | |
|---|---|---|---|---|
| Median | NC | 7.17 | 11.5 | 35.3 |
| Min, Max | 5.36, 5.36 | 3.23, 9.49 | 6.47, 17.1 | 18.0, 41.1 |

MP_{AUC}

| n | 1 | 4 | | 3 | | 5 |
|---|---|---|---|---|---|---|
| Geometric Mean | NC | 2.88 | | 2.46 | | 6.64 |
| Geometric CV | NC | 53.0 | | 118.9 | | 41.9 |
| Arithmetic Mean (SD) | NC (NC) | 3.18 | (1.70) | 3.12 | (2.08) | 7.07 (2.71) |

| | | | | |
|---|---|---|---|---|
| Median | NC | 2.64 | 3.57 | 6.65 |
| Min, Max | 4.20, 4.20 | 1.87, 5.57 | 0.846, 4.93 | 3.77, 10.9 |

MPCₘₐₓ

| n | 1 | 5 | | 3 | | 5 |
|---|---|---|---|---|---|---|
| Geometric Mean | NC | 1.51 | | 1.34 | | 2.96 |
| Geometric CV | NC | 42.9 | | 90.1 | | 65.3 |
| Arithmetic Mean (SD) | NC (NC) | 1.62 | (0.711) | 1.59 | (0.985) | 3.38 (1.84) |

| | | | | |
|---|---|---|---|---|
| Median | NC | 1.50 | 1.66 | 2.65 |
| Min, Max | 2.31, 2.31 | 0.889, 2.80 | 0.568, 2.53 | 1.27, 5.93 |

Abbreviations: CV = Coefficient of Variation, EOT = End of Treatment, Max = Maximum, Min = Minimum, MP_{AUC} = metabolite to parent ratio base on AUCₗₐₛₜ, MP_{Cmax} = metabolite to parent ratio based on Cₘₐₓ, N = Number of subjects in the respective group, n = Number of Observations, NC = Not Calculated, QD = Every Day, QOD = Every Other Day, SD = Standard Deviation. AUCₗₐₛₜ = area under the concentration-time curve from hour 0 to the last measurable concentration AUC_{0-24 h} = area under the concentration-time curve from 0 to 24 hours postdose(QOD cohorts, Week 52 only), Cₘₐₓ = maximum observed plasma concentration, Tₘₐₓ = time to maximum observed plasma concentration

TABLE 29

| | | | | |
|---|---|---|---|---|
| Parameter (unit) | 1.0 mg QD | 2.5 mg QD | 5.0 mg QOD | 10.0 mg QOD |
| Statistics | (N = 1) | (N = 5) | (N = 3) | (N = 5) |

AUCₗₐₛₜ

| n | 0 | 3 | | 0 | 4 | |
|---|---|---|---|---|---|---|
| Geometric Mean | NA | 124 | | NA | 133 | |
| Geometric CV | NA | 39.5 | | NA | 168.0 | |
| Arithmetic Mean (SD) | NA | 131 | (53.0) | NA | 231 | (294) |

| | | | | |
|---|---|---|---|---|
| Median | NA | 108 | NA | 104 |
| Min, Max | NA | 93.0, 191 | NA | 45.3, 669 |

AUC_{0-24 h}

| n | 0 | 3 | | 0 | 4 | |
|---|---|---|---|---|---|---|
| Geometric Mean | NA | 124 | | NA | 133 | |
| Geometric CV | NA | 39.4 | | NA | 168.9 | |
| Arithmetic Mean (SD) | NA | 131 | (52.9) | NA | 231 | (294) |

| | | | | |
|---|---|---|---|---|
| Median | NA | 108 | NA | 104 |
| Min, Max | NA | 93.0, 191 | NA | 44.6, 669 |

AUC_{0-48 h}

| n | 0 | 0 | 0 | 3 | |
|---|---|---|---|---|---|
| Geometric Mean | NA | NA | NA | 115 | |
| Geometric CV | NA | NA | NA | 38.2 | |
| Arithmetic Mean (SD) | NA | NA | NA | 120 | (43.0) |

| | | | | |
|---|---|---|---|---|
| Median | NA | NA | NA | 118 |
| Min, Max | NA | NA | NA | 78.7, 165 |

tₘₐₓ

| n | 0 | 3 | | 0 | 4 | |
|---|---|---|---|---|---|---|
| Geometric Mean | NA | NC | | NA | NC | |
| Geometric CV | NA | NC | | NA | NC | |
| Arithmetic Mean (SD) | NA | 4.01 | (0.02) | NA | 1.25 | (1.89) |

| | | | | |
|---|---|---|---|---|
| Median | NA | 4.00 | 0 | 0.49 |
| Min, Max | 0 | 4.00, 4.03 | 0 | 0.00, 4.00 |

Cₘₐₓ

| n | 0 | 3 | | 0 | 4 | |
|---|---|---|---|---|---|---|
| Geometric Mean | NA | 8.94 | | NA | 9.14 | |
| Geometric CV | NA | 39.5 | | NA | 211.2 | |
| Arithmetic Mean (SD) | NA | 9.41 | (3.83) | NA | 16.9 | (21.2) |

| | | | | |
|---|---|---|---|---|
| Median | NA | 7.65 | NA | 8.55 |
| Min, Max | NA | 6.77, 13.8 | NA | 2.06, 48.3 |

AR_{AUC}

| n | 0 | 3 | | 0 | 4 | |
|---|---|---|---|---|---|---|
| Geometric Mean | NA | 1.3 | | NA | 0.325 | |
| Geometric CV | NA | 62.5 | | NA | 122.5 | |
| Arithmetic Mean (SD) | NA | 1.44 | (0.756) | NA | 0.4491 | (0.573) |

| | | | | |
|---|---|---|---|---|
| Median | NA | 1.39 | NA | 0.218 |
| Min, Max | NA | 0.707, 2.22 | NA | 0.178, 1.35 |

MP_{AUC}

| n | 0 | 3 | | 0 | 4 | |
|---|---|---|---|---|---|---|
| Geometric Mean | NA | 3.63 | | NA | 3.55 | |
| Geometric CV | NA | 69.7 | | NA | 96.9 | |
| Arithmetic Mean (SD) | NA | 4.18 | (2.87) | NA | 4.54 | (3.73) |

| | | | | |
|---|---|---|---|---|
| Median | NA | 2.65 | 0 | 3.44 |
| Min, Max | NA | 2.41, 7.48 | 0 | 1.43, 9.87 |

MPCₘₐₓ

| n | 0 | 3 | | 0 | 4 | |
|---|---|---|---|---|---|---|
| Geometric Mean | NA | 1.96 | | NA | 3.12 | |
| Geometric CV | NA | 102.7 | | NA | 74.1 | |
| Arithmetic Mean (SD) | NA | 2.50 | (2.10) | NA | 3.56 | (1.54) |

| | | | | |
|---|---|---|---|---|
| Median | NA | 1.70 | NA | 4.10 |
| Min, Max | NA | 0.910, 4.88 | NA | 1.17, 4.86 |

Abbreviations: CV = Coefficient of Variation, EOT = End of Treatment, Max = Maximum, Min = Minimum, MP_{AUC} = metabolite to parent ratio base on AUCₗₐₛₜ, MP_{Cmax} = metabolite to parent ratio based on Cₘₐₓ, N = Number of subjects in the respective group, n = Number of Observations, NA = not available, QD = Every Day, QOD = Every Other Day, SD = Standard Deviation. AUCₗₐₛₜ = area under the concentration-time curve from hour 0 to the last measurable concentration, AUC_{0-24 h} or AUC_{0-48 h} = area under the concentration-time curve from 0 to 24 or 48 hours postdose(QOD cohorts, Week 52 only), Cₘₐₓ = maximum observed plasma concentration, Tₘₐₓ = time to maximum observed plasma concentration

Evidence of systemic exposure to VK2809 and VK2809A was observed in all treated participants. Geometric means of predose concentrations were several-fold greater for the active metabolite, VK2809A, than for the prodrug, VK2809. Observed steady state seemed to be achieved by Day 28. The 5 mg QOD and 2.5 mg QD arms demonstrated observable comparable profiles. The 10 mg QOD arm profile appeared to be slightly less than dose proportional to the 2.5 mg QD and 5.0 QOD arms.

The 1.0 mg QD (n=1) and 5.0 mg QOD (n=3) arm participants had small sample sizes at Day 1 and 0 participants at Day 364 at week 52. Thus, the interpretability of the data is limited.

Day 1 AUClast, AUC0-24h, and Cmax increased in an approximate dose proportional manner with respect to the 2.5 mg QD and 5.0 mg QOD arms. The 10 mg QOD arm had slightly lower AUCs than the 5.0 mg QOD arm. Higher variability was observed at the higher doses. Day 1 median tmax ranged from 1 to 2 hours with 1 participant in the 10 mg QOD arm having a value at approximately 24 hours.

Day 364 at Week 52 AUClast, and AUC0-24 h were similar with respect to the 2.5 mg QD and 10.0 mg QOD arms. The 10 mg QOD arm had a lower Cmax. Day 364 at Week 52 median tmax ranged from 1 to 2 hours across arms.

Within the 2.5 mg QD arm, little to no accumulation in AUC0-24 was noted (median=1.04). The 10 mg QOD arm appeared to have lower exposures at Day 364 at week 52 versus Day 1 (median-0.633). At Day 364 at Week 52, the lower exposures in 3 of 4 participants was unexpected and investigations of dosing, TEAEs, and assays did not provide any conclusive information regarding this outcome.

The safety and tolerability of VK2809 was assessed through the incidence and severity of AEs, vital signs, 12-lead ECGs, physical examination, potential liver toxicity events, cardiac safety monitoring, changes in bone markers, clinical laboratory parameters, and diabetic management in the Safety Population.

A summary of treatment exposure is presented in Table 30. The mean duration of exposure ranged from 1 to 403 days in the VK2809 arms to 1 to 415 days for the placebo arm. The median duration ranged from 364 to 365 days. The mean and median total cumulative dose of VK2809 are as follows:
1.0 mg QD-mean (SD) 330.53 (72.493) mg; median 359.0 mg;
2.5 mg QD-mean (SD) 790.76 (246.421) mg; median 897.5 mg;
5.0 mg QOD mean (SD) 772.16 (232.180) mg; median 880.0 mg;
10.0 mg QOD mean (SD) 1616.50 (423.051) mg; median 1795.0 mg.

Similar cumulative mean and median VK2809 was administered to participants in the 2.5 mg QD and 5.0 mg QOD VK2809 treatment arms.

Reasons for treatment interruption included AEs and others. The number of days of treatment interruption ranged from 1 to 112 in the VK2809 treatment arms versus 1 to 20 days in the placebo arm. Note that in Table 22 participants with an interruption date without a drug restart date were excluded whereas the TEAEs that led to study drug interruption in Table 22 did not exclude participants without a drug restart date. Thus, the numbers between the tables differ.

TABLE 30

| | | | | | |
|---|---|---|---|---|---|
| Placebo QD | 1.0 mg QD | 2.5 mg QD | 5.0 mg QOD | 10.0 mg QOD | All VK2809 |
| (N = 65) | (N = 17) | (N = 66) | (N = 37) | (N = 61) | (N = 181) |

Duration of Exposure (days) [1]

| n | 65 | | 17 | | 66 | | 37 | | 61 | | 181 | |
|---|---|---|---|---|---|---|---|---|---|---|---|---|
| Mean (SD) | 297.8 | (115.85) | 335.5 | (70.65) | 322.6 | (102.57) | 321.9 | (96.20) | 324.8 | (95.13) | 324.4 | (95.52) |

| | | | | | | |
|---|---|---|---|---|---|---|
| Median | 364.0 | 365.0 | 364.5 | 364.0 | 365.0 | 365.0 |
| Min, Max | 1, 415 | 135, 373 | 22, 386 | 30, 403 | 1, 372 | 1, 403 |

Total Cumulative Dose (mg) [2]

| n | 63 | | 17 | | 66 | | 37 | | 60 | | 180 | |
|---|---|---|---|---|---|---|---|---|---|---|---|---|
| Mean (SD) | 0.00 | (0.000) | 330.53 | (72.493) | 790.76 | (246.421) | 772.16 | (232.180) | 1616.50 | (423.051) | 1018.72 | (537.436) |

| | | | | | | |
|---|---|---|---|---|---|---|
| Median | 0.00 | 359.00 | 897.50 | 880.00 | 1795.00 | 905.00 |
| Min, Max | 0.0, 0.0 | 119.0, 397.0 | 55.0, 962.5 | 65.0, 995.0 | 80.0, 1950.0 | 55.0, 1950.0 |

Duration of Interruption (days) [3]

| n | 12 | | 2 | | 8 | | 7 | | 12 | | 29 | |
|---|---|---|---|---|---|---|---|---|---|---|---|---|
| Mean (SD) | 10.9 | (8.34) | 1.5 | (0.71) | 7.9 | (15.08) | 22.4 | (40.03) | 11.6 | (12.15) | 12.5 | (22.32) |

| | | | | | | |
|---|---|---|---|---|---|---|
| Median | 8.5 | 1.5 | 2.0 | 7.0 | 5.5 | 5.0 |
| Min, Max | 1, 29 | 1, 2 | 1, 45 | 1, 112 | 1, 33 | 1, 112 |

Reason for Interruption (n (%))

| | | | | | | | | | | | | |
|---|---|---|---|---|---|---|---|---|---|---|---|---|
| Adverse Event | 9 | (13.8) | 2 | (11.8) | 3 | (4.5) | 6 | (16.2) | 5 | (8.2) | 16 | (8.8) |

| | | | | | | | | | | | |
|---|---|---|---|---|---|---|---|---|---|---|---|
| Other | 4 | (6.2) | 0 | 5 | (7.6) | 1 | (2.7) | 9 | (14.8) | 15 | (8.3) |

Note:
Percentages are based on the number of subjects in the respective group.
Abbreviations: QD = daily; QOD = every other day
[1] Duration of Exposure (days) = Date of last dose or date of last blister pack returned (for subjects still on study drug) − date of first dose + 1.
[2] Cumulative Dose (mg) = Dose Group × total number of pills not returned for subjects on QD dosing regimen. For subjects on QOD dosing regimen with odd number of pills administered, cumulative dose = Dose Group × (Number of Exposure Days −1)/2 + 1). For subjects on QOD dosing regimen with even number of pills administered, cumulative dose = Dose Group × (Number of Exposure Days)/2. For subjects who had only placebo, this value was 0.
[3] Duration of Interruption is the sum of all interruption days as recorded in the EDC.

The safety and tolerability of VK2809 was measured by assessing the incidence and severity of AEs, vital signs, 12-lead ECGs, physical examination, potential liver toxicity events, and clinical laboratory parameters in the Safety Population. Note that the VK2809 1.0 mg QD and 5.0 mg QOD arms have fewer randomized participants versus the remaining VK2809 arms and placebo arms. Thus, TEAEs may appear to be more prevalent in those arms.

A summary of AEs is presented in Table 31. The number of AEs was slightly greater than the number of TEAEs because AEs were measured from the screening period to the EOS rather than from the first dose of study drug to the end of the study.

Most participants experienced a TEAE: 86.2% (796 events) in the VK2809 arms versus 78.5% (253 events) in the placebo arm. Most TEAEs were Grade 1 or 2; 771 events in the VK2809 arms versus 246 events in the placebo arm. Twenty-five Grade 3 to 5 events occurred in the VK2809 treatment arms versus 7 Grade 3 events in the placebo arm. In the VK2809 arms, 21 events were rated Grade 3, 3 were Grade 4 and 1 was Grade 5 (death). One additional participant died but was never randomized into the study. Most TEAEs were not or unlikely related to study drug treatment; 646 events in the VK2809 treatment arms versus 197 events in the placebo arm. Most TEAEs were unrelated to study procedure: 776 events in the VK2809 arms versus 45 events in the placebo arm.

TEAEs led to discontinuation of study drug in 6.1% (23 events) of participants in the VK2809 arms versus 7.7% (5 events) in the placebo arm. TEAEs that led to discontinuation of the study in 6.1% (20 events) in the VK2809 arms versus 9.2% (10 events) in the placebo arm. Interruptions in study drug occurred in 12.2% (33 events) of participants in the VK2809 arms versus 21.5% (24 events) in the placebo arm.

SAEs were experienced by a similar percentage of participants: 13 participants (7.3%, 15 events) in the VK2809 treatment arms versus 5 participants (7.7%, 6 events) in the placebo arm. In the VK2809 treatment arm, 9 participants had Grade 3 SAEs (5%, 11 events), 3 participants had Grade 4 SAEs (1.7%, 3 events), and 1 participant had a Grade 5 SAE (0.6%, 1 event). In the placebo arm, 5 participants had 1 Grade 1 (1.5%) SAE, 2 had Grade 2, and 3 Grade 3 SAEs (3.1%). Moreover, 1 participant who was screened but not randomized died and the cause of death was unknown.

TABLE 31

| | Placebo | | 1.0 mg | | | 2.5 mg | | | 5.0 mg | | | 10.0 mg | | | All | | |
|---|---|---|---|---|---|---|---|---|---|---|---|---|---|---|---|---|---|
| | QD | | QD | | | QD | | | QOD | | | QOD | | | VK2809 | | |
| | (N = 65) | | (N = 17) | | | (N = 66) | | | (N = 37) | | | (N = 61) | | | (N = 181) | | |
| | n (%) | E | n (%) | | E | n (%) | | E | n (%) | | E | n (%) | | E | n (%) | | E |
| Any AE | 51 (78.5) | 265 | 14 | (82.4) | 98 | 57 | (86.4) | 275 | 29 | (78.4) | 180 | 58 | (95.1) | 327 | 158 | (87.3) | 880 |
| Any SAE | 5 (7.7) | 6 | 1 | (5.9) | 1 | 6 | (9.1) | 6 | 2 | (5.4) | 2 | 4 | (6.6) | 6 | 13 | (7.2) | 15 |
| Any TEAEs | 51 (78.5) | 253 | 14 | (82.4) | 93 | 55 | (83.3) | 232 | 29 | (78.4) | 163 | 58 | (95.1) | 308 | 156 | (86.2) | 796 |

TEAEs by Severity [1]

| | | | | | | | | | | | | | | | | | |
|---|---|---|---|---|---|---|---|---|---|---|---|---|---|---|---|---|---|
| Grade 1 | 6 (9.2) | 117 | 2 | (11.8) | 62 | 10 | (15.2) | 104 | 3 | (8.1) | 76 | 11 | (18.0) | 163 | 26 | (14.4) | 405 |
| Grade 2 | 40 (61.5) | 129 | 11 | (64.7) | 30 | 37 | (56.1) | 119 | 21 | (56.8) | 82 | 41 | (67.2) | 135 | 110 | (60.8) | 366 |

| | | | | | | | | | | | | | | | | |
|---|---|---|---|---|---|---|---|---|---|---|---|---|---|---|---|---|
| Grade 3 | 5 (7.7) | 7 | 0 | 0 | 7 | (10.6) | 8 | 5 | (13.5) | 5 | 4 | (6.6) | 8 | 16 | (8.8) | 21 |

| | | | | | | | | | | | | | | | |
|---|---|---|---|---|---|---|---|---|---|---|---|---|---|---|---|
| Grade 4 | 0 | 0 | 0 | 0 | 1 | (1.5) | 1 | 0 | 0 | 2 | (3.3) | 2 | 3 | (1.7) | 3 |

| | | | | | | | | | | | | | | |
|---|---|---|---|---|---|---|---|---|---|---|---|---|---|---|
| Grade 5 | 0 | 0 | 1 | (5.9) | 1 | 0 | 0 | 0 | 0 | 0 | 0 | 1 | (0.6) | 1 |

TEAEs by Relationship to Study Drug

| Definitely Related | 1 (1.5) | 13 | 0 | | 0 | 0 | | 0 | 2 | (5.4) | 6 | 4 | (6.6) | 7 | 6 | (3.3) | 13 |
|---|---|---|---|---|---|---|---|---|---|---|---|---|---|---|---|---|---|
| Probably Related | 5 (7.7) | 7 | 2 | (11.8) | 2 | 2 | (3.0) | 13 | 2 | (5.4) | 2 | 4 | (6.6) | 8 | 10 | (5.5) | 25 |
| Possibly Related | 16 (24.6) | 36 | 5 | (29.4) | 20 | 11 | (16.7) | 26 | 5 | (13.5) | 12 | 16 | (26.2) | 57 | 37 | (20.4) | 115 |
| Unlikely Related | 10 (15.4) | 60 | 2 | (11.8) | 12 | 8 | (12.1) | 30 | 9 | (24.3) | 30 | 6 | (9.8) | 58 | 25 | (13.8) | 130 |
| Unrelated | 19 (29.2) | 137 | 5 | (29.4) | 59 | 34 | (51.5) | 163 | 11 | (29.7) | 113 | 28 | (45.9) | 178 | 78 | (43.1) | 513 |

TEAEs by Relationship to Study Procedure

| Definitely Related | 4 (6.2) | 5 | 1 | (5.9) | 1 | 4 | (6.1) | 6 | 3 | (8.1) | 4 | 0 | | 0 | 8 | (4.4) | 11 |
|---|---|---|---|---|---|---|---|---|---|---|---|---|---|---|---|---|---|
| Probably Related | 0 | 1 | 0 | | 0 | 0 | | 0 | 1 | (2.7) | 4 | 0 | | 0 | 1 | (0.6) | 4 |
| Possibly Related | 2 (3.1) | 2 | 1 | (5.9) | 1 | 1 | (1.5) | 1 | 0 | | 1 | 2 | (3.3) | 2 | 4 | (2.2) | 5 |
| Unlikely Related | 5 (7.7) | 22 | 2 | (11.8) | 2 | 3 | (4.5) | 3 | 4 | (10.8) | 5 | 6 | (9.8) | 33 | 15 | (8.3) | 43 |
| Unrelated | 40 (61.5) | 223 | 10 | (58.8) | 89 | 47 | (71.2) | 222 | 21 | (56.8) | 149 | 50 | (82.0) | 273 | 128 | (70.7) | 733 |
| TEAEs led to | 14 (21.5) | 24 | 3 | (17.6) | 7 | 7 | (10.6) | 7 | 6 | (16.2) | 8 | 6 | (9.8) | 11 | 22 | (12.2) | 33 |
| study drug | | | | | | | | | | | | | | | | | |
| interruption | | | | | | | | | | | | | | | | | |
| TEAEs led to DC | 5 (7.7) | 5 | 2 | (11.8) | 2 | 1 | (1.5) | 1 | 3 | (8.1) | 3 | 5 | (8.2) | 17 | 11 | (6.1) | 23 |
| of study drug | | | | | | | | | | | | | | | | | |
| TEAEs Led to | 6 (9.2) | 10 | 2 | (11.8) | 2 | 1 | (1.5) | 1 | 2 | (5.4) | 2 | 6 | (9.8) | 15 | 11 | (6.1) | 20 |
| DC of Study | | | | | | | | | | | | | | | | | |

SAEs by Severity [1]

| Grade 1 | 1 (1.5) | 1 | 0 | 0 | 0 | | 0 | 0 | | 0 | 0 | | 0 | 0 | | 0 |
|---|---|---|---|---|---|---|---|---|---|---|---|---|---|---|---|---|
| Grade 2 | 2 (3.1) | 3 | 0 | 0 | 0 | | 0 | 0 | | 0 | 0 | | 0 | 0 | | 0 |
| Grade 3 | 2 (3.1) | 2 | 0 | 0 | 5 | (7.6) | 5 | 2 | (5.4) | 2 | 2 | (3.3) | 4 | 9 | (5.0) | 11 |

| | | | | | | | | | | | | | | | |
|---|---|---|---|---|---|---|---|---|---|---|---|---|---|---|---|
| Grade 4 | 0 | 0 | 0 | 0 | 1 | (1.5) | 1 | 0 | 0 | 2 | (3.3) | 2 | 3 | (1.7) | 3 |

| | | | | | | | | | | | | | | |
|---|---|---|---|---|---|---|---|---|---|---|---|---|---|---|
| Grade 5 | 0 | 0 | 1 | (5.9) | 1 | 0 | 0 | 0 | 0 | 0 | 0 | 1 | (0.6) | 1 |

SAEs by Relationship to Study Drug

| Possibly Related | 0 | 0 | 0 | | 0 | 0 | | 0 | 0 | | 0 | 1 | (1.6) | 3 | 1 | (0.6) | 3 |
|---|---|---|---|---|---|---|---|---|---|---|---|---|---|---|---|---|---|
| Unlikely Related | 0 | 0 | 0 | | 0 | 1 | (1.5) | 1 | 0 | | 0 | 0 | | 0 | 1 | (0.6) | 1 |
| Unrelated | 5 (7.7) | 6 | 1 | (5.9) | 1 | 5 | (7.6) | 5 | 2 | (5.4) | 2 | 3 | (4.9) | 3 | 11 | (6.1) | 11 |

SAEs by relationship to study procedure

| Definitely Related | 0 | 0 | 0 | | 0 | 1 | (1.5) | 1 | 0 | | 0 | 0 | | 0 | 1 | (0.6) | 1 |
|---|---|---|---|---|---|---|---|---|---|---|---|---|---|---|---|---|---|
| Unrelated | 5 (7.7) | 6 | 1 | (5.9) | 1 | 5 | (7.6) | 5 | 2 | (5.4) | 2 | 4 | (6.6) | 6 | 12 | (6.6) | 14 |
| SAEs led to death | 0 | 0 | 1 | (5.9) | 1 | 0 | | 0 | 0 | | 0 | 0 | | 0 | 1 | (0.6) | 1 |
| Treatment-emergent | 0 | 0 | 1 | (5.9) | 1 | 0 | | 0 | 0 | | 0 | 0 | | 0 | 1 | (0.6) | 1 |
| SAEs led to death | | | | | | | | | | | | | | | | | |
| SAEs led to study | 2 (3.1) | 3 | 0 | | 0 | 1 | (1.5) | 1 | 1 | (2.7) | 1 | 0 | | 0 | 2 | (1.1) | 2 |
| drug interruption | | | | | | | | | | | | | | | | | |
| SAEs led to DC | 0 | 0 | 1 | (5.9) | 1 | 1 | (1.5) | 1 | 1 | (2.7) | 1 | 1 | (1.6) | 3 | 4 | (2.2) | 6 |
| Of study drug | | | | | | | | | | | | | | | | | |
| SAEs led to DC | 0 | 0 | 1 | (5.9) | 1 | 1 | (1.5) | 1 | 1 | (2.7) | 1 | 2 | (3.3) | 3 | 5 | (2.8) | 6 |
| of study | | | | | | | | | | | | | | | | | |

[1] Grade 1 = Mild, Grade 2 = Moderate, Grade 3 = Severe, Grade 4 = Life-threatening, and Grade 5 = Death.
Abbreviations: DC = discontinuation; E = number of events QD = daily; QOD = every other day; SAE = Serious Adverse Event, TEAE = Treatment-Emergent Adverse Event.
Notes:
TEAE is defined as an AE with start date on or after the date of first dose of study drug and up to 30 days after date of last dose of study drug. AEs occurring more than 30 days after the last treatment date were reported as TEAEs if they are assessed as related to treatment by the investigator. A treatment related AE is an AE considered by the investigator as definitely, possibly, or probably related to treatment or with unknown/missing relationship to treatment.
Missing CTCAE grade of an adverse event is imputed as Grade 4 severity. The table presents the number and percentage of subjects (n(%)) and E. Percentages are based on the number of subjects in the respective group. Subjects are counted under the most severe event and strongest relationship event for summary by severity and relationship, respectively.

A summary of TEAEs with an incidence >10% in any treatment arm is presented in Table 32. The most common SOC was Infections and Infestations with a similar number of participants: 47.5% in the VK2809 arm versus 46.2% in the placebo arm. The next most common SOC was Gastrointestinal Disorders: 33.7% versus 32.2%, respectively. Corona virus infection was the most common TEAE by preferred team in the VK2809 arms with nearly 86 participants (47.5%, 142 events). The most common TEAE in the placebo arm was fatigue in 11 participants (16.9%, 13 events).

Of note, type 2 diabetes mellitus was reported in 26 participants in the VK2809 arms with 27 TEAEs versus 4 participants with 4 TEAEs. Most of these TEAEs were worsening of existing type 2 diabetes mellitus.

Treatment-related TEAEs experienced in at least 10% of participants in any treatment group (i.e., TEAEs assessed as definitely, probably, and possible related to study drug by the investigator) are presented in Table 33. Drug-related TEAEs were reported in 53 participants (29.3%, 153 events) in the VK2809 treatment arms versus 22 participants (33.8%, 56 events). The most common drug-related TEAEs were events in the VK2809 treatment arms related to gastrointestinal disorder conditions in 15 participants (8.3%, 38 events) versus the placebo arms in 12 participants (18.5%, 18 events). Fatigue was the most common drug-related TEAEs by PT in all VK2809 and placebo arms except for the 1.0 mg QD arm: 2.5 mg QD (3 participants, 4.5%), 5.0 mg QOD (2 participants, 5.4%), and 10.0 mg QOD (6 participants, 9.8%) versus the placebo arm (7 participants, 10.8%). Headache was also the most common drug-related TEAEs by PT in the 5.0 mg QOD VK2809 arm: 5.0 QOD (2 participants, 5.4%). Diarrhea was the most common drug related TEAEs by PT in the 1.0 mg QD VK2809 (3 participants, 17.6%).

Six participants (3.3%, 16 events) in the VK2809 treatment arms and 3 participants (4.6%, 3 events) in the placebo arm had treatment-related TEAEs that led to discontinuation. In the VK2809 arms, 4 participants had 14 events in the 10.0 mg arm, and 1 participant had 1 event each in the 1.0 mg QD and 5.0 mg QOD arms. No TEAE led to discontinuation in the 2.5 mg QD VK2809 treatment arm. One participant in the 10.0 mg QOD VK2809 had 3 serious treatment-related TEAEs; depression, homicidal and suicidal ideations that led to discontinuation. Most TEAEs were unrelated to study procedure: 776 events in the VK2809 arms versus 45 events in the placebo arm. Twenty events were related to study procedure in the VK2809 arms versus 7 events in the placebo arm.

Most TEAEs were rated Grade 1 or Grade 2. Twenty participants in the VK2809 treatment arms versus 5 participants in the placebo arm had TEAEs rated Grade 3 or higher. All events in the placebo arm were Grade 3. In the VK2809 arm, 16 participants had 21 Grade 3 events, 3 participants had 3 Grade 4 events: 2 in the 10.0 mg QOD arm and 1 in 5.0 mg QOD arm, and 1 participant had Grade 5 event in the 1.0 mg QD arm. All Grades 3 to 5 TEAEs were unique except Grade 3 syncope in 2 participants in VK2809 arms.

Most treatment-related TEAEs were rated as Grade 1 or Grade 2 in severity. Five treatment-related TEAEs were Graded 3 or 4. Four participants, 2 in the VK2809 5.0 mg QOD arm versus 2 participants in the placebo arm had Grade 3 TEAEs. One participant in the VK2809 10.0 mg QOD arm had 1 Grade 4, and 2 Grade 3 treatment related TEAEs.

TABLE 32

| | Placebo | | | 1.0 mg | | | 2.5 mg | | | 5.0 mg | | | 10.0 mg | | | All | | |
|---|---|---|---|---|---|---|---|---|---|---|---|---|---|---|---|---|---|---|
| | QD | | | QD | | | QD | | | QOD | | | QOD | | | VK2809 | | |
| System Organ Class | (N = 65) | | | (N = 17) | | | (N = 66) | | | (N = 37) | | | (N = 61) | | | (N = 181) | | |
| Preferred Term | n (%) | | E | n (%) | | E | n (%) | | E | n (%) | | E | n (%) | | E | n (%) | | E |
| Any TEAE | 51 | (78.5) | 253 | 14 | (82.4) | 93 | 55 | (83.3) | 232 | 29 | (78.4) | 163 | 58 | (95.1) | 308 | 156 | (86.2) | 796 |
| Infections & | 29 | (44.6) | 47 | 7 | (41.2) | 11 | 28 | (42.4) | 46 | 16 | (43.2) | 25 | 29 | (47.5) | 49 | 80 | (44.2) | 131 |
| infestations | | | | | | | | | | | | | | | | | | |
| Corona virus | 7 | (10.8) | 8 | 4 | (23.5) | 5 | 12 | (18.2) | 12 | 8 | (21.6) | 8 | 10 | (16.4) | 10 | 34 | (18.8) | 35 |
| infection | | | | | | | | | | | | | | | | | | |
| Urinary tract | 7 | (10.8) | 9 | 0 | | 0 | 6 | (9.1) | 10 | 5 | (13.5) | 5 | 5 | (8.2) | 6 | 16 | (8.8) | 21 |
| infection | | | | | | | | | | | | | | | | | | |
| Gastrointestinal | 21 | (32.3) | 36 | 7 | (41.2) | 15 | 15 | (22.7) | 40 | 12 | (32.4) | 26 | 24 | (39.3) | 46 | 58 | (32.0) | 127 |
| disorders | | | | | | | | | | | | | | | | | | |
| Nausea | 7 | (10.8) | 8 | 2 | (11.8) | 2 | 6 | (9.1) | 10 | 6 | (16.2) | 9 | 8 | (13.1) | 9 | 22 | (12.2) | 30 |
| Diarrhoea | 4 | (6.2) | 5 | 4 | (23.5) | 4 | 6 | (9.1) | 6 | 2 | (5.4) | 2 | 7 | (11.5) | 12 | 19 | (10.5) | 24 |
| Abdominal pain | 0 | | 0 | 3 | (17.6) | 4 | 3 | (4.5) | 5 | 2 | (5.4) | 2 | 3 | (4.9) | 3 | 11 | (6.1) | 14 |
| Investigations | 8 | (12.3) | 9 | 4 | (23.5) | 22 | 13 | (19.7) | 27 | 5 | (13.5) | 7 | 18 | (29.5) | 28 | 40 | (22.1) | 84 |
| General disorders | 16 | (24.6) | 23 | 4 | (23.5) | 8 | 16 | (24.2) | 21 | 11 | (29.7) | 16 | 21 | (34.4) | 35 | 52 | (28.7) | 80 |
| & administration | | | | | | | | | | | | | | | | | | |
| site conditions | | | | | | | | | | | | | | | | | | |
| Fatigue | 11 | (16.9) | 13 | 2 | (11.8) | 2 | 8 | (12.1) | 8 | 3 | (8.1) | 4 | 9 | (14.8) | 10 | 22 | (12.2) | 24 |
| Oedema peripheral | 2 | (3.1) | 2 | 0 | | 0 | 3 | (4.5) | 3 | 7 | (18.9) | 8 | 3 | (4.9) | 4 | 13 | (7.2) | 15 |
| Asthenia | 1 | (1.5) | 1 | 2 | (11.8) | 2 | 1 | (1.5) | 1 | 0 | | 0 | 1 | (1.6) | 1 | 4 | (2.2) | 4 |
| Musculoskeletal & | 14 | (21.5) | 23 | 3 | (17.6) | 4 | 13 | (19.7) | 17 | 7 | (18.9) | 9 | 20 | (32.8) | 35 | 43 | (23.8) | 65 |
| connective tissue | | | | | | | | | | | | | | | | | | |
| disorders | | | | | | | | | | | | | | | | | | |
| Muscle spasms | 0 | | 0 | 1 | (5.9) | 1 | 3 | (4.5) | 3 | 1 | (2.7) | 1 | 7 | (11.5) | 9 | 12 | (6.6) | 14 |
| Nervous system | 10 | (15.4) | 10 | 2 | (11.8) | 7 | 12 | (18.2) | 14 | 11 | (29.7) | 17 | 17 | (27.9) | 27 | 42 | (23.2) | 65 |
| disorders | | | | | | | | | | | | | | | | | | |
| Headache | 2 | (3.1) | 2 | 1 | (5.9) | 2 | 3 | (4.5) | 3 | 5 | (13.5) | 6 | 10 | (16.4) | 11 | 19 | (10.5) | 22 |
| Dizziness | 3 | (4.6) | 3 | 2 | (11.8) | 2 | 4 | (6.1) | 4 | 3 | (8.1) | 3 | 7 | (11.5) | 10 | 16 | (8.8) | 19 |
| Metabolism & | 12 | (18.5) | 15 | 7 | (41.2) | 10 | 9 | (13.6) | 11 | 11 | (29.7) | 15 | 16 | (26.2) | 19 | 43 | (23.8) | 55 |
| nutrition disorders | | | | | | | | | | | | | | | | | | |
| Type 2 DM | 4 | (6.2) | 4 | 2 | (11.8) | 2 | 6 | (9.1) | 6 | 6 | (16.2) | 7 | 12 | (19.7) | 12 | 26 | (14.4) | 27 |
| Gout | 1 | (1.5) | 1 | 2 | (11.8) | 5 | 1 | (1.5) | 1 | 1 | (2.7) | 1 | 1 | (1.6) | 1 | 5 | (2.8) | 8 |
| Skin & | 7 | (10.8) | 11 | 2 | (11.8) | 2 | 11 | (16.7) | 15 | 4 | (10.8) | 5 | 8 | (13.1) | 11 | 25 | (13.8) | 33 |
| subcutaneous | | | | | | | | | | | | | | | | | | |
| tissue disorders | | | | | | | | | | | | | | | | | | |
| Respiratory, | 15 | (23.1) | 20 | 3 | (17.6) | 3 | 6 | (9.1) | 10 | 6 | (16.2) | 8 | 7 | (11.5) | 11 | 22 | (12.2) | 32 |
| thoracic & | | | | | | | | | | | | | | | | | | |
| mediastinal | | | | | | | | | | | | | | | | | | |
| disorders | | | | | | | | | | | | | | | | | | |
| Dyspnoea | 4 | (6.2) | 5 | 1 | (5.9) | 1 | 2 | (3.0) | 2 | 5 | (13.5) | 5 | 3 | (4.9) | 4 | 11 | (6.1) | 12 |
| Injury, poisoning | 8 | (12.3) | 12 | 3 | (17.6) | 4 | 9 | (13.6) | 10 | 5 | (13.5) | 8 | 4 | (6.6) | 6 | 21 | (11.6) | 28 |
| & procedural | | | | | | | | | | | | | | | | | | |
| complications | | | | | | | | | | | | | | | | | | |
| Procedural pain | 3 | (4.6) | 3 | 2 | (11.8) | 2 | 1 | (1.5) | 1 | 3 | (8.1) | 4 | 0 | | 0 | 6 | (3.3) | 7 |
| Cardiac disorders | 10 | (15.4) | 13 | 3 | (17.6) | 3 | 2 | (3.0) | 2 | 5 | (13.5) | 7 | 6 | (9.8) | 8 | 16 | (8.8) | 20 |

| | | | | | | | | | | | | | | | | | |
|---|---|---|---|---|---|---|---|---|---|---|---|---|---|---|---|---|---|
| Renal & urinary | 4 | (6.2) | 5 | 0 | 0 | 3 | (4.5) | 4 | 4 | (10.8) | 4 | 5 | (8.2) | 5 | 12 | (6.6) | 13 |
| disorders | | | | | | | | | | | | | | | | | |
| Vascular disorders | 5 | (7.7) | 5 | 0 | 0 | 4 | (6.1) | 4 | 4 | (10.8) | 4 | 4 | (6.6) | 4 | 12 | (6.6) | 12 |

Notes:
TEAEs are defined as AEs with start date on or after the date of first dose of study drug and up to 30 days after date of last dose of study drug. AEs occurring more than 30 days after the last treatment date are also reported as TEAEs if they are assessed to be related to treatment by the investigator. If a subject has multiple occurrences of an AE, the subject is presented only once in the respective subject count. The table presents the number and percentage of subjects (n (%)) and number of events (E). Percentages are based on the number of subjects in the respective group. AEs are coded using MedDRA Version 22.1.
Abbreviations: AEs = adverse events; DM = diabetes mellitus; E = number of events QD = daily; QOD = every other day; SAE = Serious Adverse Event, TEAE = treatment-emergent adverse event

TABLE 33

| | Placebo QD | | | 1.0 mg QD | | | 2.5 mg QD | | 5.0 mg QOD | | | 10.0 mg QOD | | | All VK2809 | | |
|---|---|---|---|---|---|---|---|---|---|---|---|---|---|---|---|---|---|
| System Organ Class | (N = 65) | | | (N = 17) | | | (N = 66) | | (N = 37) | | | (N = 61) | | | (N = 181) | | |
| Preferred Term | n (%) | | E | n (%) | | E | n (%) | E | n (%) | | E | n (%) | | E | n (%) | | E |
| Any Treatment- | 22 | (33.8) | 56 | 7 | (41.2) | 22 | 13 (19.7) | 39 | 9 | (24.3) | 20 | 24 | (39.3) | 72 | 53 | (29.3) | 153 |
| related TEAE | | | | | | | | | | | | | | | | | |
| Gastrointestinal | 12 | (18.5) | 18 | 4 | (23.5) | 9 | 3 (4.5) | 15 | 1 | (2.7) | 4 | 7 | (11.5) | 10 | 15 | (8.3) | 38 |
| disorders | | | | | | | | | | | | | | | | | |
| Nausea | 5 | (7.7) | 5 | 2 | (11.8) | 2 | 2 (3.0) | 5 | 1 | (2.7) | 2 | 3 | (4.9) | 3 | 8 | (4.4) | 12 |
| Diarrhoea | 2 | (3.1) | 3 | 3 | (17.6) | 3 | 2 (3.0) | 2 | 1 | (2.7) | 1 | 3 | (4.9) | 3 | 9 | (5.0) | 9 |
| General disorders and | 9 | (13.8) | 11 | 2 | (11.8) | 2 | 4 (6.1) | 6 | 3 | (8.1) | 3 | 10 | (16.4) | 15 | 19 | (10.5) | 26 |
| administration site | | | | | | | | | | | | | | | | | |
| conditions | | | | | | | | | | | | | | | | | |
| Fatigue | 7 | (10.8) | 8 | 0 | | 0 | 3 (4.5) | 3 | 2 | (5.4) | 2 | 6 | (9.8) | 7 | 11 | (6.1) | 12 |
| Nervous system | 4 | (6.2) | 4 | 1 | (5.9) | 3 | 2 (3.0) | 3 | 3 | (8.1) | 5 | 8 | (13.1) | 11 | 14 | (7.7) | 22 |
| disorders | | | | | | | | | | | | | | | | | |
| Investigations | 1 | (1.5) | 1 | 1 | (5.9) | 4 | 3 (4.5) | 5 | 2 | (5.4) | 2 | 8 | (13.1) | 8 | 14 | (7.7) | 19 |

Notes:
TEAE is defined as an AE with start date on or after the date of first dose of study drug and up to 30 days after date of last dose of study drug.
If a subject has multiple occurrences of an AE, the subject is presented only once in the respective subject count. The table presents the number and percentage of subjects (n (%)) and E. Percentages are based on the number of subjects in the respective group. AEs are coded using MedDRA Version 22.1.
Abbreviations: AE = adverse event; E = number of events QD = daily; QOD = every other day; SAE = Serious Adverse Event, TEAE = treatment-emergent adverse event

Most participants in all treatment arms had TEAEs: 156 participants (86.2%) in the VK2809 arms versus 51 participants (78.5%) in the placebo arm. Most TEAEs were Grade 1 or 2 in both treatment arms and were not related to treatment or study procedures. Grade 3 to Grade 5 TEAEs were experienced by 25 (13.8%) participants in the VK2809 arms versus 7 (10.7%) participants with Grade 3 TEAEs in the placebo arm. Drug-related TEAEs were reported in 53 participants (29.3%, 153 events) in the VK2809 treatment arms versus 22 participants (33.8%, 56 events).

SAEs were experienced by 13 participants (7.2%) in the VK2809 arms versus 5 participants 7.7% in the placebo arm. Moreover, 10 participants had 12 SAEs during the screening period that were never randomized into the study. Two participants died; 1 participant who was never randomized and died of an unknown cause and 1 participant in the VK2809 1.0 mg arm who died of a coronavirus infection.

TEAEs that led to discontinuation of study drug were experienced by 11 (6.1%) participants in VK2809 arms versus 5 participants (7.7%) participants in the placebo arm. TEAEs that led to discontinuation of study were experienced by 11 (6.1%) participants in VK2809 arms versus 6 (9.2%) participants in the placebo arm. TEAEs that led to drug interruptions were experienced in 22 (12.2%) participants in the VK2809 arms versus 14 (21.5%) participants in the placebo arm.

The most common TEAE was corona virus infection experienced by 86 (18.8%) of participants in the VK2809 arms and fatigue experienced by 11 participants (16.9%) in the placebo arm. Fatigue was the most common drug-related TEAE: 11 participants (6.1%) in the VK2809 arms versus 7 participants (10.8%) in the placebo arm.

This Phase 2, multicenter, multi-country study of VK2809 in participants diagnosed with NASH/MASH provided data to meet the study objectives.

Between the VK2809 treatment arms, efficacy results were similar but more pronounced with increasing VK2809 dose. For the primary endpoint, participants in the VK2809 arms versus placebo experienced statistically significant reductions in liver fat content as assessed by MRI-PDFF. Notably up to 75% of participants had ≥30% relative reduction in liver fat content.

For the secondary endpoint of NASH/MASH resolution with no worsening of fibrosis, participants in the VK2809 arms demonstrated NASH/MASH resolution 68.6% versus 29.3% for placebo (p<0.215) as assessed by liver biopsy. For the secondary endpoint ≥1 stage improvement in fibrosis and no worsening of steatohepatitis, 51.1% of participants in the VK2809 arms demonstrated improved fibrosis versus 34.1% for placebo (p<0.0497 in the higher 3 dose arms) as assessed by liver biopsy. For the secondary endpoint of both resolution of NASH/MASH and improvement of fibrosis, 43.8% of participants in the VK2809 arms demonstrated improved fibrosis versus 19.5% for placebo (p≤0.0206 in the higher 3 dose arms) as assessed by liver biopsy.

Study results were consistent after sensitivity analyses were used. A more conservative analysis, which categorized participants with missing data as non-responders, produced similar statistical outcomes.

For sparse sampling, evidence of systemic exposure to VK2809 and VK2809A was observed in all treated participants. Geometric means of predose concentrations were several-fold greater for the active metabolite, VK2809A, than for the for the prodrug, VK2809. Observed steady state seemed to be achieved by Day 28.

For intensive sampling, Day 1 AUClast, AUC0-24h, and Cmax increased in an approximate dose proportional manner with respect to the 2.5 mg QD and 5.0 mg QOD arms. The 10.0 mg QOD arm had slightly lower AUCs than the 5.0 mg QOD arm. Day 1 median tmax ranged from 1 to 2 hours in all but 1 participant. Day 364 at Week 52 AUClast and AUC0-24 h were similar with respect to the 2.5 mg QD and 10.0 mg QOD arms. The 10.0 mg QOD arm had a lower Cmax. Week 52 median tmax ranged from 1 to 2 hours across all arms.

Within the 2.5 mg QD arm, little to no accumulation in AUC0-24 was noted (median=1.04). The 10.0 mg QOD arm appeared to have lower exposures at Day 364 at week 52 versus Day 1 (median=0.633). At Week 52, the lower exposures in 3 of 4 participants was unexpected and investigations of dosing, TEAEs, and assays did not provide any conclusive information regarding this outcome.

VK2809 demonstrated safety and tolerability in this study to 52 weeks of treatment. Most TEAEs were reported as mild or moderate. The most common TEAE in the VK2809 arms was coronavirus infection versus fatigue for participants treated with placebo. TEAEs that led to discontinuation were balanced among treatment arms. Two participants died: 1 who was never randomized, and another of coronavirus (VK2809 arm). SAEs were experienced by 13 participants in the VK2809 arms versus 5 participants in the placebo arm. Moreover, 10 participants had 12 SAEs during the screening period that were never randomized into the study.

Laboratory, vital signs, and ECG differences in mean change from baseline over time in laboratory values between treatment arms were similar across treatment groups. Most changes were small, transient, and not clinically meaningful.

Overall, VK2809 achieved primary endpoints demonstrating robust reduction in liver fat at 12 weeks and maintained at 52 weeks. Histologic endpoints demonstrated NASH/MASH resolution, improvement in fibrosis, and combination of both at 52 weeks. Results showed significant reductions in plasma lipids LDL-C, triglycerides, Lp(α), Apo(β), and Apo(C)-III. There was excellent tolerability rate of GI-related side effects were similar to placebo. There was promising safety with 94% of AEs being classified as mild to moderate.

Various exemplary aspects are set forth below.
1. A method for statistically significantly reducing and/or maintaining liver fat for longer than a twelve-week time period, comprising administering an amount effective for said statistically reducing and/or maintaining of a pharmaceutical composition to a human during a time the human is in need of such significant reduction and/or maintenance in liver fat, the pharmaceutical composition comprising an amorphous form of VK2809; and a pharmaceutically acceptable carrier.
2. The method of aspect 1, wherein the amount effective ranges from 1 mg up to and including 2.5 mg of the pharmaceutical composition and wherein said administering occurs once every day.
3. The method of aspect 1, wherein the amount effective ranges from greater than 2.5 mg up to and including 4 mg of the pharmaceutical composition and wherein said administering occurs once every day.
4. The method of aspect 3, wherein the amount effective ranges from 3 mg up to and including 3.5 mg of the pharmaceutical composition and wherein said administering occurs once every day.
5. The method of aspect 4, wherein the amount effective is 3.25 mg of the pharmaceutical composition and wherein said administering occurs once every day.
6. The method of aspect 1, wherein the amount effective ranges from 1 mg up to and including 10 mg of the pharmaceutical composition and wherein said administering occurs once every day.
7. The method of aspect 1, wherein the amount effective ranges from 5 mg up to and including 10 mg of the pharmaceutical composition and wherein said administering occurs once every other day.
8. The method of aspect 7, wherein the amount effective is 10 mg of the pharmaceutical composition and wherein said administering occurs once every other day.
9. The method of aspect 1, wherein the pharmaceutically acceptable carrier is chosen from at least one polyethylene glycol compound.
10. The method of aspect 1, wherein said liver fat is reduced and/or maintained by at least 30% up to and including 60%.
11. The method of aspect 1, wherein the liver fat reduction and/or maintenance is measured as a mean hepatic fat fraction.
12. The method of aspect 11, wherein said mean hepatic fat fraction is measured using a magnetic resonance imaging derived proton density fat fraction.
13. The method of aspect 1, wherein said time is for fifty-two weeks.
14. A method for statistically significantly treating and/or improving fibrosis, comprising administering an amount effective for said statistically treating and/or improving of a pharmaceutical composition to a human during a time the human is in need of such significant treatment and/or improvement in fibrosis, the pharmaceutical composition comprising an amorphous form of VK2809; and a pharmaceutically acceptable carrier.
15. The method of aspect 14, wherein the amount effective ranges from 1 mg up to and including 2.5 mg of the pharmaceutical composition and wherein said administering occurs once every day.
16. The method of aspect 14, wherein the amount effective ranges from greater than 2.5 mg up to and including 4 mg of the pharmaceutical composition and wherein said administering occurs once every day.
17. The method of aspect 16, wherein the amount effective ranges from 3 mg up to and including 3.5 mg of the pharmaceutical composition and wherein said administering occurs once every day.
18. The method of aspect 17, wherein the amount effective is 3.25 mg of the pharmaceutical composition and wherein said administering occurs once every day.
19. The method of aspect 14, wherein the amount effective ranges from 1 mg up to and including 10 mg of the pharmaceutical composition and wherein said administering occurs once every day.
20. The method of aspect 14, wherein the amount effective ranges from 5 mg up to and including 10 mg of the pharmaceutical composition and wherein said administering occurs once every other day.
21. The method of aspect 20, wherein the amount effective is 10 mg of the pharmaceutical composition and wherein said administering occurs once every other day.
22. The method of aspect 14, wherein the pharmaceutically acceptable carrier is chosen from at least one polyethylene glycol compound.
23. The method of aspect 14, wherein said treatment and/or improvement to fibrosis is measured using a liver biopsy.
24. The method of aspect 14, wherein said time is for fifty-two weeks.
25. A method for statistically significantly treating and/or improving non-alcoholic fatty liver disease and/or NASH/MASH, comprising administering an amount effective for said statistically treating and/or improving of a pharmaceutical composition to a human during a time the human is in need of such significant treatment and/or improvement in non-alcoholic fatty liver disease and/or NASH/MASH, the pharmaceutical composition comprising an amorphous form of VK2809; and a pharmaceutically acceptable carrier.
26. The method of aspect 25, wherein the amount effective ranges from 1 mg up to and including 2.5 mg of the pharmaceutical composition and wherein said administering occurs once every day.
27. The method of aspect 25, wherein the amount effective ranges from greater than 2.5 mg up to and including 4 mg of the pharmaceutical composition and wherein said administering occurs once every day.
28. The method of aspect 27, wherein the amount effective ranges from 3 mg up to and including 3.5 mg of the pharmaceutical composition and wherein said administering occurs once every day.
29. The method of aspect 28, wherein the amount effective is 3.25 mg of the pharmaceutical composition and wherein said administering occurs once every day.
30. The method of aspect 25, wherein the amount effective ranges from 1 mg up to and including 10 mg of the pharmaceutical composition and wherein said administering occurs once every day.
31. The method of aspect 25, wherein the amount effective ranges from 5 mg up to and including 10 mg of the pharmaceutical composition and wherein said administering occurs once every other day.
32. The method of aspect 31, wherein the amount effective is 10 mg of the pharmaceutical composition and wherein said administering occurs once every other day.
33. The method of aspect 25, wherein the pharmaceutically acceptable carrier is chosen from at least one polyethylene glycol compound.
34. The method of aspect 25, wherein said treatment and/or improvement to non-alcoholic fatty liver disease and/or NASH/MASH is measured using a liver biopsy.
35. The method of aspect 25, wherein said time is for fifty-two weeks.
36. A method for reducing and/or maintaining liver fat for longer than a twelve-week time period, comprising administering a pharmaceutical composition to a human during a time the human is in need of such reduction and/or maintenance in liver fat, the composition comprising a means for reducing and/or maintaining liver fat in said human in need; and a pharmaceutically acceptable carrier.
37. The method of aspect 36, wherein said time is for fifty-two weeks.
38. A method for treating and/or improving fibrosis, comprising administering a pharmaceutical composition to a human during a time the human is in need of such treatment and/or improvement of fibrosis, the composition comprising a means for treating and/or improving fibrosis in said human in need; and a pharmaceutically acceptable carrier.
39. The method of aspect 38, wherein said time is for fifty-two weeks.
40. A method for treating and/or improving non-alcoholic fatty liver disease and/or NASH/MASH, comprising administering a pharmaceutical composition to a human during a time the human is in need of such treatment and/or improvement of non-alcoholic fatty liver disease and/or NASH/MASH, the composition comprising a means for treating and/or improving non-alcoholic fatty liver disease and/or NASH/MASH in said human in need; and a pharmaceutically acceptable carrier.
41. The method of aspect 40, wherein said time is for fifty-two weeks.

1-13. (canceled)

14. A method for treating a human patient having non-alcoholic fatty liver disease, NASH/MASH, and/or liver fibrosis, the method comprising administering a pharmaceutical composition to the human patient every day or every other day,
wherein the pharmaceutical composition comprises from 1 mg to 10 mg of an amorphous form of VK2809 and a pharmaceutically acceptable carrier, and
wherein said treatment results in one or more of the following in said patient:
(i) at least a one-stage improvement in fibrosis score without worsening of NASH/MASH;
(ii) at least a one-stage improvement in fibrosis with resolution of NASH/MASH;
(iii) at least a two-point improvement in NAS score without worsening of fibrosis; or
(iv) a NAS inflammation score of 0-1 and a ballooning score of 0 with no worsening of fibrosis.

15. The method of claim 14, wherein the pharmaceutical composition comprises from 1 mg up to and including 2.5 mg of VK2809, and said administering occurs once every day.

16. The method of claim 14, wherein the pharmaceutical composition comprises from greater than 2.5 mg up to and including 4 mg of VK2809, and said administering occurs once every day.

17. The method of claim 16, wherein the pharmaceutical composition comprises from 3 mg up to and including 3.5 mg of VK2809, and said administering occurs once every day.

18. The method of claim 17, wherein the pharmaceutical composition comprises 3.25 mg of VK2809, and said administering occurs once every day.

19. The method of claim 14, wherein said administering occurs once every day.

20. The method of claim 14, wherein the pharmaceutical composition comprises from 5 mg up to and including 10 mg of VK2809, and said administering occurs once every other day.

21. The method of claim 20, wherein the pharmaceutical composition comprises 10 mg of VK2809, and said administering occurs once every other day.

22. The method of claim 14, wherein the pharmaceutically acceptable carrier is chosen from at least one polyethylene glycol compound.

23. The method of claim 14, wherein one or more of (i)-(iv) is achieved by fifty-two weeks after initiating said treatment.

24. The method of claim 14, wherein said treatment further results in a reduction in NIS4 and/or PRO-C3.

25-28. (canceled)

29. A method for treating a human patient having non-alcoholic fatty liver disease, NASH/MASH, and/or liver fibrosis, the method comprising administering VK2809 to the human patient every day or every other day an amount effective to achieve, by fifty-two weeks after initiation of said treatment, one or more of the following in said human patient:
(i) at least a one-stage improvement in fibrosis score without worsening of NASH/MASH;
(ii) at least a one-stage improvement in fibrosis with resolution of NASH/MASH;
(iii) at least a two-point improvement in NAS score without worsening of fibrosis; or
(iv) a NAS inflammation score of 0-1 and a ballooning score of 0 with no worsening of fibrosis.

30. The method of claim 29, wherein administering an effective amount of VK2809 comprises administering from 1 mg to 10 mg every day or every other day.

31. The method of claim 30, comprising administering from 1 mg up to and including 5 mg of VK2809 to the patient every day.

32. The method of claim 30, comprising administering from 1 mg up to and including 2.5 mg of VK2809 to the patient every day.

33. The method of claim 30, comprising administering from 2.5 mg up to and including 4 mg of VK2809 to the patient every day.

34. The method of claim 30, comprising administering from 3 mg up to and including 3.5 mg of VK2809 to the patient every day.

35. The method of claim 30, comprising administering from 5 mg up to and including 10 mg of VK2809 to the patient every other day.

36. The method of claim 30, comprising administering 10 mg of VK2809 to the patient every other day.
